SEQUENCE LISTING (33 sequences)

SEQ ID NO: 1 - protein, 20 aa, synthetic construct
KETAAAKFERQHMDSSTSAA

SEQ ID NO: 2 - protein, 19 aa, synthetic construct
KETNWAWFWDQHMDSSTSA

SEQ ID NO: 3 - protein, 19 aa, synthetic construct
KETGWALFVQQHMDSSTSA

SEQ ID NO: 4 - protein, 19 aa, synthetic construct
KETVMANFQMQHMDSSTSA

SEQ ID NO: 5 - protein, 19 aa, synthetic construct
KETGDAVFARQHMDSSTSA

SEQ ID NO: 6 - protein, 19 aa, synthetic construct
KETGWAAFVKQHMDSSTSA

SEQ ID NO: 7 - protein, 19 aa, synthetic construct
KETGWATFVEQHMDSSTSA

SEQ ID NO: 8 - protein, 19 aa, synthetic construct
KETKLAFFLKQHMDSSTSA

SEQ ID NO: 9 - protein, 19 aa, synthetic construct
KETWWAWFFGQHMDSSTSA

SEQ ID NO: 10 - protein, 19 aa, synthetic construct
KETTWAEFTWQHMDSSTSA

SEQ ID NO: 11 - protein, 19 aa, synthetic construct
KETPWASFNKQHMDSSTSA

SEQ ID NO: 12 - protein, 19 aa, synthetic construct
KETAMAMFVTQHMDSSTSA

SEQ ID NO: 13 - protein, 19 aa, synthetic construct
KETLWAWFMWQHMDSSTSA

SEQ ID NO: 14 - protein, 15 aa, synthetic construct
KETAAAKFERQHMDS

SEQ ID NO: 15 - protein, 15 aa, synthetic construct
KETAAAKFERQHMNS

SEQ ID NO: 16 - protein, 15 aa, synthetic construct
NRAWSEFLWQHLAPV

SEQ ID NO: 17 - protein, 15 aa, synthetic construct
NRGWSEFLWQHHAPV

SEQ ID NO: 18 - protein, 15 aa, synthetic construct
NRAWSVFQWQHIAPA

SEQ ID NO: 19 - protein, 105 aa, synthetic construct
MSSSNYCNQMMKSRNLTKDRCKPVNTFVHESLADVQAVCSQKNVACKNGQTNCYQSYSTMSITDCRETGSSKYPNCAYKTTQANKHIIVACEGNPYVPVHFDASV

SEQ ID NO: 20 - protein, 102 aa, synthetic construct
MSSSNYCNQMMKSRNLTKDRCKPVNTFVHESLADVQAVCSQKNVACKNGQTNCYQSYSTMSITDCRETGSSKYPNCAYKTTQANKHIIVACEGNPYVPVHFD

SEQ ID NO: 21 - protein, 124 aa, Bos taurus
KETAAAKFERQHMDSSTSAASSSNYCNQMMKSRNLTKDRCKPVNTFVHESLADVQAVCSQKNVACKNGQTNCYQSYSTMSITDCRETGSSKYPNCAYKTTQANKHIIVACEGNPYVPVHFDASV

SEQ ID NO: 22 - DNA, 348 bp, synthetic construct
  source 1..348: mol_type = other DNA
ggtaccatgagcagctccaactactgtaaccagatgatgaagagccggaacctgaccaaagatcgatgcaagccagtgaacacctttgtgcacgagtccctggctgatgtccaggccgtgtgctcccagaaaaatgttgcctgcaagaatgggcagaccaattgctaccagagctactccaccatgagcatcaccgactgccgtgagaccggcagctccaagtaccccaactgtgcctacaagaccacccaggcgaataaacacatcattgtggcttgtgagggaaacccgtacgtgccagtccactttgatgcttcagtgcatcaccatcaccatcactagggatcc

SEQ ID NO: 23 - protein, 111 aa, unidentified
MSSSNYCNQMMKSRNLTKDRCKPVNTFVHESLADVQAVCSQKNVACKNGQTNCYQSYSTMSITDCRETGSSKYPNCAYKTTQANKHIIVACEGNPYVPVHFDASVHHHHHH

SEQ ID NO: 24 - DNA, 552 bp, synthetic construct
  source 1..552: mol_type = other DNA
ggtaccatgggcgcctccaggctctataccctggtgctggtcctgcagcctcagcgagttctcctgggcatgaaaaagcgaggcttcggggccggccggtggaatggctttgggggcaaagtgcaagaaggagagaccatcgaggatggggctaggagggagctgcaggaggagagcggtctgacagtggacgccctgcacaaggtgggccagatcgtgtttgagttcgtgggcgagcctgagctcatggacgtgcatgtcttctgcacagacagcatccaggggacccccgtggagagcgacgaaatgcgcccatgctggttccagctggatcagatccccttcaaggacatgtggcccgacgacagctactggtttccactcctgcttcagaagaagaaattccacgggtacttcaagttccagggtcaggacaccatcctggactacacactccgcgaggtggacacggtcaaggaaactgcagcagccaagtttgagcggcagcacatggactccagcacttccgctgcctaggctgcctagggatcc

SEQ ID NO: 25 - protein, 176 aa, synthetic construct
MGASRLYTLVLVLQPQRVLLGMKKRGFGAGRWNGFGGKVQEGETIEDGARRELQEESGLTVDALHKVGQIVFEFVGEPELMDVHVFCTDSIQGTPVESDEMRPCWFQLDQIPFKDMWPDDSYWFPLLLQKKKFHGYFKFQGQDTILDYTLREVDTVKETAAAKFERQHMDSSTSAA

SEQ ID NO: 26 - DNA, 561 bp, synthetic construct
  source 1..561: mol_type = other DNA
ggtaccatgggcgcctccaggctctataccctggtgctggtcctgcagcctcagcgagttctcctgggcatgaaaaagcgaggcttcggggccggccggtggaatggctttgggggcaaagtgcaagaaggagagaccatcgaggatggggctaggagggagctgcaggaggagagcggtctgacagtggacgccctgcacaaggtgggccagatcgtgtttgagttcgtgggcgagcctgagctcatggacgtgcatgtcttctgcacagacagcatccaggggacccccgtggagagcgacgaaatgcgcccatgctggttccagctggatcagatccccttcaaggacatgtggcccgacgacagctactggtttccactcctgcttcagaagaagaaattccacgggtacttcaagttccagggtcaggacaccatcctggactacacactccgcgaggtggacacggtcgccgccgccaaggaaactgcagcagccaagtttgagcggcagcacatggactccagcacttccgctgcctaggctgcctagggatcc

SEQ ID NO: 27 - protein, 179 aa, synthetic construct
MGASRLYTLVLVLQPQRVLLGMKKRGFGAGRWNGFGGKVQEGETIEDGARRELQEESGLTVDALHKVGQIVFEFVGEPELMDVHVFCTDSIQGTPVESDEMRPCWFQLDQIPFKDMWPDDSYWFPLLLQKKKFHGYFKFQGQDTILDYTLREVDTVAAAKETAAAKFERQHMDSSTSAA

SEQ ID NO: 28 - DNA, 582 bp, synthetic construct
  source 1..582: mol_type = other DNA
ggtaccatgggcgcctccaggctctataccctggtgctggtcctgcagcctcagcgagttctcctgggcatgaaaaagcgaggcttcggggccggccggtggaatggctttgggggcaaagtgcaagaaggagagaccatcgaggatggggctaggagggagctgcaggaggagagcggtctgacagtggacgccctgcacaaggtgggccagatcgtgtttgagttcgtgggcgagcctgagctcatggacgtgcatgtcttctgcacagacagcatccaggggacccccgtggagagcgacgaaatgcgcccatgctggttccagctggatcagatccccttcaaggacatgtggcccgacgacagctactggtttccactcctgcttcagaagaagaaattccacgggtacttcaagttccagggtcaggacaccatcctggactacacactccgcgaggtggacacggtcgccgccgccgccgccgccgccgccgccgccaaggaaactgcagcagccaagtttgagcggcagcacatggactccagcacttccgctgcctaggctgcctagggatcc

SEQ ID NO: 29 - protein, 186 aa, synthetic construct
MGASRLYTLVLVLQPQRVLLGMKKRGFGAGRWNGFGGKVQEGETIEDGARRELQEESGLTVDALHKVGQIVFEFVGEPELMDVHVFCTDSIQGTPVESDEMRPCWFQLDQIPFKDMWPDDSYWFPLLLQKKKFHGYFKFQGQDTILDYTLREVDTVAAAAAAAAAAKETAAAKFERQHMDSSTSAA

SEQ ID NO: 30 - DNA, 2181 bp, synthetic construct
  source 1..2181: mol_type = other DNA
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgcaaggaaactgcagcagccaagtttgagcggcagcacatggactccagcacttccgctgcctga

SEQ ID NO: 31 - protein, 726 aa, synthetic construct
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKACKETAAAKFERQHMDSSTSAA

SEQ ID NO: 32 - DNA, 3693 bp, synthetic construct
  source 1..3693: mol_type = other DNA
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcatgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggcgggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcaaaggaaactgcagcagccaagtttgagcggcagcacatggactccagcacttccgctgcctga

SEQ ID NO: 33 - protein, 1230 aa, synthetic construct
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLIMQLMPFGCLLDYVREHKDNIGSQYLLNWCVQIAKGMNYLEDRRLVHRDLAARNVLVKTPQHVKITDFGRAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGAKETAAAKFERQHMDSSTSAA

(19) United States
(12) Patent Application Publication
(10) Pub. No.: US 2023/0204594 A1
(43) Pub. Date: Jun. 29, 2023

(54) SYSTEMS AND METHODS FOR REAL-TIME CELLULAR DRUG-TARGET ENGAGEMENT

(71) Applicant: NERD BIO LLC, SAN DIEGO, CA (US)

(72) Inventors: Ivan BABIC, SAN DIEGO, CA (US); Elmar NURMAMMADOV, SAN DIEGO, CA (US)

(21) Appl. No.: 18/056,440

(22) Filed: Nov. 17, 2022

Related U.S. Application Data
(60) Provisional application No. 63/280,287, filed on Nov. 17, 2021.

(51) Int. Cl. G01N 33/58; C12Q 1/6851; G16B 25/20; G16B 15/30; G01N 33/557

(52) U.S. Cl. CPC G01N 33/582; C12Q 1/6851; G16B 25/20; G16B 15/30; G01N 33/557

(57) ABSTRACT

Provided herein are methods and systems allowing for real-time measurement of cellular drug-target engagement. Use of fluorescence-based cell target engagement technology with real-time gene expression machinery provides several advantages over prior systems. Integration with real-time gene expression machinery without bias to any particular design or brand is provided. Programmability of cell target engagement methodology, such that any software in real-time gene expression machine can seamlessly be used for programming is provided. Single or multiple machine integration for the use of cell target engagement technology is provided. Compatibility with multiple multi-well plate setups is provided. Development of unique modifications of real-time (quantitative) gene expression software to detect real-time cellular drug-target engagement to work efficiently with existing gene expression machinery is provided.

### RELATED APPLICATION

This application claims priority to U.S. Provisional Application Ser. No. 63/280,287, filed Nov. 17, 2021, the disclosure of which is hereby incorporated by reference in its entirety.

### REFERENCE TO AN ELECTRONIC SEQUENCE LISTING

The contents of the electronic sequence listing (062554-0572109_SEQUENCE_LISTING.xml; Size: 40,590 bytes; and Date of Creation: Nov. 14, 2022) is herein incorporated by reference in its entirety.

### FIELD OF THE INVENTION

Embodiments of the invention related to systems and methods for real-time cellular drug-target engagement.

### SUMMARY

Methods and systems provided herein are based, in part, on a modified type of enzyme complementation assay that requires the assembly of two components, a nuclease acceptor and a nuclease donor, that can assemble into a function nuclease complex capable of cleaving a labeled nucleic acid substrate. In some embodiment, the methods and systems provided herein comprise a acceptor peptide and a donor protein, that can assemble into a functional RNase complex capable of cleaving a labeled nucleic acid substrate. The acceptor peptide is provided as a fusion protein comprising the acceptor peptide (S-tag acceptor peptide) and a target polypeptide of interest (e.g., a viral coat protein). The assay is conducted, in certain embodiments, in the presence of a test compound and a denaturant (e.g., heat) that denatures the fusion protein and prevents assembly of an active RNase complex. If a test compound interacts with and/or binds to the target polypeptide and inhibits denaturation of the fusion protein, an active RNase complex is formed and cleavage of the labeled substrate can be detected, thereby identifying a potential drug candidate (i.e., a test compound) that interacts with the target polypeptide (e.g., a viral coat protein).

One aspect of the present disclosure relates to a method of determining if a test compound can interact with a target polypeptide. The method comprises (a) contacting a fusion protein with (i) a test compound, (ii) a denaturant, (iii) a ribonuclease (RNase) acceptor, and (iv) a nucleic acid substrate; and (b) detecting an amount of a cleavage product of the nucleic acid substrate; wherein the fusion protein comprises a target polypeptide and an S-tag acceptor peptide. In certain embodiments, the contacting of (a) comprises contacting one or more cells with one or more of (i)-(iv). In certain embodiments, the contacting of (a) comprises contacting a cell lysate with one or more of (i)-(iv). In some embodiments, the cell or lysate comprises the fusion protein. In some embodiments, the substrate is a FRET-labeled nucleic acid substrate. In some embodiments, the nucleic acid substrate comprises a pair of FRET labels. In some embodiments, the amount of the cleavage product comprises detecting an amount of a fluorescence signal emitted from the cleavage product and obtaining data points, and the fluorescence signal allows for the identification of a target saturation dose, the apparent equilibrium dissociation constant (K_{D}), the half maximal effective concentration (EC50) of target engagement, between the target polypeptide and the test compound.

In some embodiments, the method is conducted in at least 100 separate vessels, substantially simultaneously, wherein the test compound in each of the separate vessels is a different test compound.

Another aspect of the present disclosure relates to a method of determining if a test compound can interact with a target polypeptide. The method comprises: (a) preparing a reaction solution enabling contact of a fusion protein within a system comprising: (i) a test compound or vehicle. (ii) a denaturant. (iii) a nuclease donor. (iv) a nucleic acid substrate, and/or (v) a signal controller; and (b detecting an amount and speed of a cleavage product of the nucleic acid substrate in real time by use of a machine configured to detect fluorescence, generated light, or derivative thereof.

In some embodiments, the machine is a machine capable of measuring light, fluorescence, or derivative thereof. In some embodiments, the machine is a quantitative polymerase chain reaction (qPCR) or a quantitative reverse transcription polymerase chain reaction (RT-qPCR) machine. In some embodiments, the qPCR or RT-qPCR machine is programmed to mix, initiate, amplify signal, register signal, deconvolute data, analyze data, obtained from the reaction solution in real time, wherein the program comprises any of: a) mixing reaction solution in batch wherein all ingredients are added from start of mixing, or by injection mode wherein ingredients added at various times; b) running reaction modules, each with their own commands, such that the data generated in one module can automatically define commands of the next module; c) running singular or multiplexed reactions; d) running kinetic series, where various combinations of temperatures and compound doses are tested without prior knowledge of target polypeptide's thermal profile; e) performing variously timed incubation or incubations with or without agitation or excitation; f) ramping temperature linearly, step-wise regularly, or irregularly, wherein each ramp step is defined with its own temperature range, speed, repeats and temperature/signal ratios; g) introducing alternating steps of temperature incubation, signal excitation, and pause; h) performing data registration, amplification, conversion, deconvolution, and/or analysis; i) communicating data within a local or remote machine circuit; and/or j) parsing generated meta-data using programmed analysis methods to identify signal patterns.

In some embodiments, the machine generates target engagement data configured to facilitate the multi-dimensional determination and quantification of engagement between the test compound and the target polypeptide. In some embodiments, the target engagement data is configured to facilitate identification of binding stoichiometry, target occupancy, residence time, KD, K-on, K-off, and EC50. In some embodiments, the machine is programmed to generate the target engagement data, and the program comprises any of: a) mixing reaction solution in batch wherein all ingredients are added from start of mixing, or by injection mode wherein ingredients added separately and/or at various times; b) running reaction modules, each with their own commands, such that the data generated in one module can automatically define commands of the next module; c) running singular or multiplexed reactions; d) running kinetic series, where various combinations of temperatures and compound doses are tested without prior knowledge of target polypeptide's thermal profile; e) performing variously timed incubation or incubations with or without agitation or excitation; f) ramping temperature linearly, step-wise regularly, or irregularly, wherein each ramp step is defined with its own temperature range, speed, repeats and temperature/signal ratios; g) introducing alternating steps of temperature incubation, signal excitation, and pause; h) performing data registration, amplification, conversion, deconvolution, and/or analysis; i) communicating data within a local or remote machine circuit; and/or j) parsing generated meta-data using programmed analysis methods in order to find signal patterns.

In some embodiments, the fusion protein comprises: (a) the target polypeptide and nuclease acceptor domain, (b) the target polypeptide and a nuclease, or (c) the target polypeptide, an N-terminal domain of a nuclease and a first domain allowing for dimerization of the N-terminal domain to a C-terminal domain of the same nuclease fused to a second domain complementary to the domain allowing for dimerization. In some embodiments, the nuclease acceptor domain of (a) is an S-tag acceptor peptide, and wherein the nuclease is an S-protein. In some embodiments, a split-Cas9 system is designed where the nuclease domain and the helical domain are cloned and expressed independently, then complemented in a controlled reaction. This enables a more controlled nuclease activity of the Cas9 enzyme. Either of these domains or their sub-domains, can be fused with a target polypeptide, then complemented with the rest of the enzyme in a cellular target engagement system (Wright A V et al. “Rational design of a split-Cas9 enzyme complex.” Proceedings of the National Academy of Sciences of the United States of America vol. 112.10 (2015): 2984-9. doi:10.1073/pnas.1501698112). In some embodiments, the nuclease of (b) is selected from the group consisting of Cas9, Micrococcal nuclease, Rnase H, a non-natural nuclease hybrid such as Cas9-Fok1, and Cpf1/Cas12a. In some embodiments, the nuclease of (c) is Cas9, the first domain allowing for dimerization is Coh2, and the second domain is DocS. Coh2 and DocS are two C. thermocellum proteins that interact with high affinity. A Coh2-DocS complementation system can be designed where either of these proteins, or domains or sub-domains thereof, is fused with a target polypeptide, then complemented with the rest of the Coh2-DocS complex in a cellular target engagement system (Yu Y et al. Engineering a far-red light-activated split-Cas9 system for remote-controlled genome editing of internal organs and tumors. Sci Adv. 2020; 6(28):eabb1777. Published 2020 Jul. 10. doi:10.1126/sciadv.abb1777).

In some embodiments, the signal controller is any optional compound or excitant that may control the enzymatic activity of the complemented active enzyme, control the start of the target engagement reaction, control the speed of this reaction, and/or control the duration/maturity of the reaction. In some embodiments, the signal controller is an antibody, chemical, peptide, temperature, LV, microwave, or light. In some embodiments, the signal controller is far-red light. In some embodiments, binding of the Coh2 and DocS domains is enabled by a signal controller, wherein the signal controller is far-red light.

In some embodiments, parts or the entirety of reaction solution is/are prepared outside or inside of the machine, such that: the entirety of reaction is prepared inside the machine; parts of reaction are prepared outside of the machine, then transferred into the machine; and/or certain reaction components are injected into the machine at once or sequentially.

In some embodiments, the machine communicates in a circuit with other machines connected locally or remotely.

In some embodiments, the solution is held within a container or a vessel compatible with real-time fluorescence measuring, and wherein the machine is programmed to read fluorescence signals from the reaction solution in real time.

In some embodiments, the container or vessel is a tube or a multi-well plate compatible with real-time fluorescence measuring.

In some embodiments, the multi-well plate comprises a microfluidic chip enabling reaction multiplexing. In some embodiments, multiplexing is achieved with a microfluidic chip which mixes all or only desired combinations from two sets of liquids. In some embodiments, the first set of liquids is a panel of various tagged target polypeptides, and the second set of liquids is a panel of various drug or their doses. In some embodiments, multiplexing is achieved without a microfluidic chip, where the combinatorial power is achieved by virtue of differential liquid dispensing, either before uploading or otherwise inputting into the detector or qPCR machine, or within it. In some embodiments, a liquid dispenser might be able to dispense various doses from a panel of drugs into a micro-plate comprising more than a thousand wells, carrying various target polypeptides, thus generating multiple combinations. Such chip-independent multiplexing is limited to the reaction and/or well capacity of the microplate to carry such combinations.

Another aspect of the present disclosure relates to a to non-transitory computer readable medium having instructions thereon. The instructions when executed by a computer, a processor, a machine that includes the computer and/or the processor, and/or other systems, cause the computer, the processor, the machine, and/or other systems to perform any of the operations in the methods described above.

Another aspect of the present disclosure relates to a machine configured to determine if a test compound can interact with a target polypeptide. The machine comprises one or more processors configured by machine readable instructions to: (a) facilitate preparation of a reaction solution enabling contact of a fusion protein within a system comprising: (i) a test compound or vehicle, (ii) a denaturant, (iii) a nuclease acceptor, (iv) a nuclease acceptor. (v) a nucleic acid substrate, and/or (vi) a signal controller; and (b) detect an amount and speed of a cleavage product of the nucleic acid substrate in real time by use of a machine configured to detect fluorescence, generated light, or derivative thereof.

In some embodiments, the machine is a machine capable of measuring light, fluorescence, or derivative thereof. In some embodiments, the machine is a qPCR or a RT-qPCR machine. In some embodiments, the qPCR or RT-qPCR machine is programmed to mix, initiate, amplify signal, register signal, deconvolute data, analyze data, obtained from the reaction solution in real time, wherein the program comprises any of: a) mixing reaction solution in batch wherein all ingredients are added from start of mixing, or by injection mode wherein ingredients added at various times; b) running reaction modules, each with their own commands, such that the data generated in one module can automatically define commands of the next module; c) running singular or multiplexed reactions; d) running kinetic series, where various combinations of temperatures and compound doses are tested without prior knowledge of target polypeptide's thermal profile; el performing variously timed incubation or incubations with or without agitation or excitation; f) ramping temperature linearly, step-wise regularly, or irregularly, wherein each ramp step is defined with its own temperature range, speed, repeats and temperature/signal ratios; g) introducing alternating steps of temperature incubation, signal excitation, and pause; h) performing data registration, amplification, conversion, deconvolution, and/or analysis; i) communicating data within a local or remote machine circuit; and/or j) parsing generated meta-data using programmed analysis methods to identify signal patterns.

In some embodiments, the machine generates target engagement data configured to facilitate the multi-dimensional determination and quantification of engagement between the test compound and the target polypeptide. In some embodiments, the target engagement data is configured to facilitate identification of binding stoichiometry, target occupancy, residence time, KD, K-on, K-off, and EC50.

In some embodiments, the machine is programmed to generate the target engagement data, and the program comprises any of: a) mixing reaction solution in batch wherein all ingredients are added from start of mixing, or by injection mode wherein ingredients added separately and/or at various times; b) running reaction modules, each with their own commands, such that the data generated in one module can automatically define commands of the next module: c) running singular or multiplexed reactions; d) running kinetic series, where various combinations of temperatures and compound doses are tested without prior knowledge of target polypeptide's thermal profile; e) performing variously timed incubation or incubations with or without agitation or excitation; f) ramping temperature linearly, step-wise regularly, or irregularly, wherein each ramp step is defined with its own temperature range, speed, repeats and temperature/signal ratios; g) introducing alternating steps of temperature incubation, signal excitation, and pause; h) performing data registration, amplification, conversion, deconvolution, and/or analysis; i) communicating data within a local or remote machine circuit; and/or j) parsing generated meta-data using programmed analysis methods in order to find signal patterns.

In some embodiments, the fusion protein comprises the target polypeptide and an S-tag acceptor peptide.

In some embodiments, parts or the entirety of reaction solution is/are prepared outside or inside of the machine, such that: the entirety of reaction is prepared inside the machine; parts of reaction are prepared outside of the machine, then transferred into the machine; and/or certain reaction components are injected into the machine at once or sequentially.

In some embodiments, the machine communicates in a circuit with other machines connected locally or remotely. In some embodiments, the solution is held within a container or a vessel compatible with real-time fluorescence measuring, and wherein the machine is programmed to read fluorescence signals from the reaction solution in real time.

In some embodiments, the container or vessel is a tube or a multi-well plate compatible with real-time fluorescence measuring.

In some embodiments, the multi-well plate comprises a microfluidic chip enabling reaction multiplexing.

Certain aspects of the technology are described further in the following description, examples, claims and drawings.

In some embodiments, the nucleic acid substrate comprises more than one nucleotide long. In some embodiments, the nucleic acid substrate comprises natural and/or non-natural nucleotides. In some embodiments, the nucleic acid substrate comprises nucleotides connected by cleavable bonds.

In some embodiments, a nucleic acid substrate comprises a suitable fluorescence energy transfer (FRET) label. One advantage of FRET-labeled nucleic acids is that they can readily enter an intact cell thereby allowing detection of cleavage of the substrate in whole cells. In certain embodiments, a nucleic acid substrate comprises a FRET label comprising a suitable fluorescent donor/acceptor pair separated by polynucleotide comprising an nuclease cleavable sequence such that fluorescence emission of the donor is quenched until the substrate is cleaved by a nuclease, or by a assembled nuclease complex.

In some embodiments, the nucleic acid substrate further comprises fluorophores suitable for FRET labeling. In some embodiments, the nucleic acid substrate comprises two fluorophores (fluorescent labels), one at each end of the nucleic acid (i.e., a first fluorophore at its 3′ end, and a second fluorophore at its 5′ end). Non-limiting examples of a fluorescent label include fluorescein, rhodamine, Texas Red, phycoerythrin, allophycocyanin, 6-carboxyfluorescein (6-FAM), 2,7-dimethoxy-4′,5′-dichloro-6-carboxyfluorescein (JOE), 6-carboxy-X-rhodamine (ROX), 6-carboxy-2′,4′,7,4,7-hexachlorofluorescein (HEX), 5-carboxyfluorescein (5-FAM) or N.N.N′,N′-tetramethyl-6-carboxyrhodamine (TAMRA), cyanine dyes, such as Cy3, Cy5, Alexa 542, Bodipy 630/650, fluorescent particles, fluorescent semiconductor nanocrystals, the like, and combinations thereof. In some embodiments, the two fluorophores are FAM and TAMRA, where FAM is linked to the 3′ end of the nucleic acid TAMRA is linked to the 5′ end of the nucleic acid.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 illustrates series of operations for a method for real-time or near real time cellular drug-target engagement.

FIG. 2 shows a system for real time or near real time cellular drug-target engagement.

FIG. 3 shows an example computer system that may be used to perform one or more of the operations described herein.

FIG. 4 shows a schematic illustration of an embodiment of an S-tag complementation strategy.

FIG. 5 shows an exemplary time course signal development of fluorescence for HEK293 cells transfected with DNA encoding for MTH1-Stag fusion, then lysed and complemented with nuclease substrate and S-protein, in which the fluorescence is measured immediately upon lysis and complementation.

FIG. 6 shows an exemplary time course signal development of fluorescence for HEK293 cells transfected with DNA encoding for MTH1-Stag fusion, then lysed and complemented with nuclease substrate and S-protein, in which the fluorescence is measured 24 hours after lysis and complementation.

FIG. 7 shows an exemplary time-course signal development of fluorescence for HEK293 cells transfected with DNA encoding for MTH1-Stag fusion, lysed and complemented with nuclease substrate and S-protein, in which the fluorescence is measured immediately upon lysis and complementation, and compared to a blank buffer.

FIG. 8 shows an exemplary real-time course signal development of fluorescence for HEK293 cells transfected with DNA encoding the indicated proteins having Stag fusion, then lysed and heated at the indicated temperatures spanning 38° C. to 62° C., then complemented with nuclease substrate+S-protein. Measurement of fluorescence signal was started immediately in the RT-QPCR machine after the reaction was mixed.

FIG. 9 shows the thermal profile for each of the indicated proteins having Stag fusions from FIG. 8. The fluorescence detected at the linear part of the curves from FIG. 8 were plotted v temperature from 38° C. to 62° C. to generate a thermal profile that identifies a temperature of aggregation (T_{agg}).

FIG. 10 shows an exemplary time-course signal development of fluorescence for HEK293 cells were transfected with DNA encoding for MTH1-Stag fusion, then lysed and complemented with nuclease substrate+S-protein. Measurement of fluorescence signal was started immediately in RT-QPCR machine after the reaction was mixed. Temperature was ramped from 25° C. to 83° C. with 3° C. increments.

FIG. 11 shows an exemplary time-course signal development of fluorescence for HEK293 cells transfected with DNA encoding for MTH1-Stag fusion, then lysed and complemented with nuclease substrate and S-protein, in which the fluorescence is measured immediately upon lysis and complementation. Signal of the complete reaction (MTH1_Stag expressing cell lysate+S protein+Substrate) was compared to several controls: MTH1-Stag only, Substrate only, S protein only, Substrate+S protein only, MTH1-Stag+Substrate only, Lysate+S protein only.

FIG. 12 shows an exemplary temperature challenge for HEK293 cells transfected with DNA encoding for MTH1-Stag fusion, then lysed and complemented with nuclease substrate and S-protein, in which the fluorescence is measured immediately upon lysis and complementation. Signal of the complete reaction (MTH1_Stag expressing cell lysate+S protein+Substrate) was compared to several controls: MTH1-Stag only, Substrate only, S protein only, Substrate+S protein only, MTH1-Stag+Substrate only, Lysate+S protein only. Temperature was ramped from 25° C. to 83° C. with 5° C. increments.

FIG. 13 shows an exemplary temperature challenge for HEK293 cells transfected with DNA encoding for MTH1-Stag fusion, then lysed and complemented with nuclease substrate and S-protein, in which the fluorescence is measured immediately upon lysis and complementation. 25° C. incubation was followed by temperature ramping from 25° C. to 81° C. with 3° C. increments.

FIG. 14 shows an exemplary time-course signal development of fluorescence and temperature challenge with a Crizotinib dose range. HEK293 cells expressing MTH1-Stag fusion protein were treated with Crizotinib in dose range of 10.000, 2000, 400, 80, 16, 3.2, 0.64, 0 nM for 1 hr. The cells were then lysed and complemented with nuclease substrate+S-protein. Measurement of fluorescence signal was started immediately in RT-QPCR machine after the reaction was mixed. 25° C. incubation was followed by temperature ramping from 25° C. to 81° C. with 3° C. increments.

FIG. 15 shows an exemplary time-course signal development of fluorescence with a Crizotinib dose range. HEK293 cells expressing MTH1-Stag fusion protein were treated with Crizotinib in dose range of 10.000, 2000, 400, 80, 16, 3.2, 0.64, 0 nM for 1 hr. The cells were then lysed and complemented with nuclease substrate+S-protein. Measurement of fluorescence signal was started immediately in RT-QPCR machine after the reaction was mixed. Reaction was subjected to temperature challenge at 59° C.

FIG. 16 shows an exemplary time-course signal development of fluorescence with a Crizotinib dose range. HEK293 cells expressing MTH1-Stag fusion protein were treated with Crizotinib in dose range of 10.000, 2000, 400, 80, 16, 3.2, 0.64, 0 nM for 1 hr. The cells were then lysed and complemented with nuclease substrate+S-protein. Measurement of fluorescence signal was started immediately in RT-QPCR machine after the reaction was mixed with a 25° C. incubation followed by a 25° C.-82° C. temperature challenge with 2° C. increments.

FIG. 17 shows an exemplary time-course HEK293 cell were transfected with DNA encoding for MTH1-Stag fusion. 96-well RT-QPCR reaction plate was used to set up the target engagement reactions. HEK293 cells expressing MTH1-Stag fusion protein were treated with Crizotinib in dose range of 10.000, 2000, 400, 80, 16, 3.2, 0.64, 0 nM for 1 hr. The cells were then lysed and complemented with nuclease substrate+S-protein. Measurement of fluorescence signal was started immediately in RT-QPCR machine after the reaction was mixed, with a 25° C. incubation followed by a 45° C.-70° C. temperature challenge with 5° C. temperature increments, followed by incubation at 25° C.

FIG. 18 shows an exemplary time-course with temperature ramping and a Crizotinib dose range. HEK293 cells expressing MTH1-Stag fusion protein were treated with Crizotinib in dose range of 10.000, 2000, 400, 80, 16, 3.2, 0.64, 0 nM for 1 hr. The cells were then lysed and complemented with nuclease substrate+S-protein. Measurement of fluorescence signal was started immediately in RT-QPCR machine after the reaction was mixed, then, temperature was ramped 25° C.-83° C. with 2° C. ramping for signal high resolution in a single run, then followed by incubation at 25° C.

FIG. 19 shows an exemplary real-time course signal development of fluorescence for HEK293 cells transfected with DNA encoding MTH1 Stag fusion, then lysed and treated with either (S) or (R) Crizotinib at the indicated concentrations ranging from 2000 nM to 0.1 nM and immediately heated at 54° C. The fluorescence detected from the linear part of the real-time curves was plotted with concentration of the drug on semi-log and non-linear regression analysis used to fit a sigmoidal dose response curve to identify an EC50 of target engagement.

FIG. 20 shows a chemical structure of one embodiment of an optimized fluorogenic substrate, 6-FAM-dArUdAdA-6-TAMRA, where 6-FAM refers to 6-carboxyfluorescein and 6-TAMRA refers to 6-carboxy-tetramethylrhodamine. The “scissile bond” that is cleaved by active RNase S upon complementation with S-tag is identified.

FIG. 21 shows a schematic illustration of fluorescence light emission as a result of cleavage of a fluorogenic substrate by an assembled RNase donor (A)-protein-S-tag acceptor peptide (D) complex.

FIG. 22 shows an immunoblot showing expression of an exemplary in HEK-293 cells where the fusion protein comprises an S-tag acceptor peptide (S-tag) and a target polypeptide (MTH1) with or without an optional 3 amino acid linker 3aa) or 10 amino acid linker (10aa).

FIG. 23 show a comparison of fluorescence signal (y-axis) resulting from fusion proteins having no linker (MTH1-Stag) or different sized linkers (MTH1-3aa-Stag or MTH1-10 aa-Stag) as a function of temperature (x-axis). In this assay, the S-tag alone (i.e., an S-tag not incorporated into a fusion protein) fails to produce any signal. Without being limited to theory, it may be that the S-tag peptide is thermodynamically unstable and therefore lacks a thermal melting profile under heat challenge when not incorporated into a fusion protein. This property makes contribution of the S-tag to the fused target protein minimal, and also makes it ideal for target engagement studies.

FIG. 24 shows a relationship between the amount of RNA donor (S Protein, x-axis) and fluorescence signal (y-axis) for different fusion protein constructs.

FIG. 25 shows the results of a dilution test of different cell numbers (legend) showing fluorescence signal (Y-axis) as a function of time (x-axis).

FIG. 26 shows the results of increasing temperature (x-axis) on fluorescent signal (y-axis) to identify a temperature of aggregation (T_{agg}).

FIG. 27 shows the results of increasing inhibitor (test compound) concentration (x-axis) on fluorescent signal (y-axis).

FIG. 28 shows the results of increasing concentration of an inhibitor (y-axis) on protein stabilization (y-axis) at 50° C.

FIG. 29 shows the effect of incubation time (legend) on signal separation as a function of temperature (x-axis).

FIG. 30 shows the effect of incubation time (legend) on fluorescence signal (y-axis) as a function of inhibitor (test compound) concentration (x-axis) using 50 ug lysate.

FIG. 31 shows the effect of incubation time (legend) on fluorescence signal (y-axis) as a function of inhibitor (test compound) concentration (x-axis) using 10 ug lysate.

FIG. 32 shows the effect of the amount of lysate (legend) on fluorescence signal (y-axis) as a function of inhibitor (test compound) concentration (x-axis) for a 0.5 minute incubation.

FIG. 33 shows the effect of the amount of lysate (legend) on fluorescence signal (y-axis) as a function of inhibitor (test compound) concentration (x-axis) for 5 minute incubation.

FIG. 34 shows the effect of the amount of lysate (legend) on fluorescence signal (y-axis) as a function of inhibitor (test compound) concentration (x-axis) for 10 minute incubation.

FIG. 35 shows an exemplary real-time monitoring of the fluorescence signal development over time for the assay. A kinetic trace of the development of the S-tag (Micro-tag) fluorescence signal over the course of 20 minutes. The kinetics of the signal was a result of the enzyme-catalyzed reaction of cleavage of the FRET-labeled substrate after enzyme complementation of the nuclease donor (S protein) and target polypeptide (MTH1) fused to the nuclease acceptor (S-tag).

FIGS. 36A-36C show exemplary thermal profiles of several micro-tagged (S-tag) fusion protein constructs reveals unique heat signatures for these tagged proteins. FIG. 36A shows that EGFR micro-tagged protein in thermal challenge identified a temperature of maximum signal (Tₘₐₓ) of 59° C. FIG. 36B shows MTH1 micro-tagged protein in thermal challenge identified a temperature of aggregation (T_{agg}) of 55° C. FIG. 36C shows BCL6 micro-tagged protein in thermal challenge identified a temperature of minimum signal (Tₘᵢₙ) of 46.5° C.

FIG. 37 shows an exemplary fluorescence signal over time (relative light units (RLU) per minute) for MTH1 micro-tag (S-tag) fusion protein heated at T_{agg} temperature or not heated. The sample that was not heated rapidly developed fluorescence signal that peaked within the first 3 minutes followed by a decrease in fluorescence signal development resulting from the depletion (by cleavage) of the FRET-labeled substrate. The heated sample had less micro-tag protein (MTH1-3aa-Stag fusion protein) and showed slower depletion (cleavage) of the substrate.

FIGS. 38A-38B show an exemplary testing of BCL6-Micro-tag (BCL6-S-tag fusion protein) with BI-3812 and BI-5273 inhibitors. Micro-tagged BCL6 was expressed in HEK293 cells and lysates from these cells were treated with the inhibitor (FIG. 38A) BI-3812 and the inactive analog (FIG. 38B) BI-5273. Samples were heated at the Tₘᵢₙ and the fluorescence signal (relative light units) detected after a 4-minute incubation with the S protein (nuclease donor) and the FRET-labeled substrate.

FIGS. 39A-39C show that, after 15 minutes, the peak of fluorescence identified the target saturation dose. FIG. 39A shows examination, after 15 minutes, of the reaction shown in FIG. 38A to identify the target saturation dose. FIG. 39B shows that removing the data points above the saturating dose allowed Sigmoidal Dose-response curve fitting to identify the EC50 of Target Engagement that is the same as that identified at the early time point (in FIGS. 38A-38C). FIG. 39C shows that the observable fluorescence signal data could also be fit to a Saturation Binding Equation (One-site total) using GraphPad Prism to identify an Apparent Equilibrium Dissociation Constant (apparent K_{D}) for the drug binding to the protein target.

FIGS. 40A-40E show an exemplary identification of a target saturation dose, Emax (maximum effect (maximum fluorescence signal)), EC50 of target engagement, and apparent K_{D}. FIG. 40A shows a kinetic trace showing fluorescence over time for each concentration of inhibitor tested. FIG. 40B shows that, at higher drug concentrations, the later time point (5 min) had lower signal than the early time point (0 min, first detection at start of the kinetic trace). This decreased signal at higher concentrations of drug resulted in a bell-shaped curve. FIG. 40C shows a bell-shaped curve identifying the saturating concentration of drug (target saturation dose) that gave maximum effect (Emax). FIG. 40D shows that EC50 of target engagement could be determined from fitting a Sigmoidal-Dose Response curve to the early time point data. FIG. 40E shows that a saturation binding curve can be generated from the identification of the target saturation dose, and that a nonlinear regression analysis of curve fitting could identify an apparent equilibrium dissociation constant K_{D}. The EC50 of Target Engagement and Apparent K_{D} were identical, demonstrating that the fluorescence readout was directly proportional to drug binding and could be used to determine apparent affinity binding constants.

FIGS. 41A-41C shows an exemplary identification of target saturation dose, Emax, EC50 of target engagement, and apparent K_{D} for (S) Crizotinib binding to MTH1 Micro-tagged protein. FIG. 41A shows EC50 of target engagement from fluorescence detection after 2 minutes of the enzyme complementation reaction after the MTH1 micro-tagged protein (MTH1-3aa-S-tag fusion protein) was heated at 55° C. in the presence of increasing concentrations of the inhibitor (S) Crizotinib. FIG. 41B shows fluorescence detection of the reaction after 10 minutes and bell-shaped curve fitting to identify target saturation does and Emax. FIG. 41C shows saturation binding curve fitting (One Site-Total) using GraphPad Prism to determine the apparent K_{D}.

### DETAILED DESCRIPTION

Micro-tag cell target engagement technology for real-time measurement of cellular drug-target engagement in real-time gene expression readers, particularly qPCR/RT-qPCR machines is described. Fluorescence-based S-tag/S-protein (Micro-tag) cellular target engagement methodology is utilized. With the present systems and methods, enhanced sensitivity and data extraction is enabled for Micro-tag cell target engagement methodology, such that early time-point signals of drug-target engagement are efficiently detected and recorded. The early points of cell target engagement were previously missed in standard plate readers due to the rapid nature of signal development; i.e. manual intervention of the user was slow enough to miss these early points of cell target engagement. The present systems and methods enable efficient capturing and measurement of cell target engagement signals from the early moments to its saturation/completion.

The nature of the fluorescence signal (its half-life, rate of decay, and level/speed of target saturation) generated due to drug-target engagement in the cell enables measurement real-time cellular drug-target engagement kinetics. The kinetics responds to abundance of the target, dosage of the drug and duration of reaction. As a result, important kinetics data such as KD, K-on, K-off and EC50 can be extracted.

Use of fluorescence-based cell target engagement technology with real-time gene expression machinery provides several advantages over prior systems (this example list is not exhaustive). Integration with real-time (quantitative) gene expression machinery without bias to any particular design or brand is provided. Programmability of cell target engagement methodology, such that any software in real-time gene expression machine can seamlessly be used for programming is provided, which also enables measurement of important kinetics data such as KD, K-on, K-off and EC50. Single or multiple machine integration for the use of cell target engagement technology is provided. For example, integration with compound pinning, liquid dispensing etc., machines is possible. This enhances work flow scalability and high-throughput drug screening. Integration with the multiplexed real-time gene expression setup of Fluidigm and/or other similar set ups is possible. This enables measurement of combinations of various plated drugs versus targets in a single plate. Compatibility with multiple multi-well plate setups is provided, particularly with Fluidigm's plates and/or other similar plates. This enables measurement of combinations of various plated drugs versus targets in a single plate.

Development of unique modifications of real-time (quantitative) gene expression software to detect real-time cellular drug-target engagement to work efficiently with existing gene expression machinery is provided. This enables efficient measurement of real-time cellular drug-target engagement kinetics.

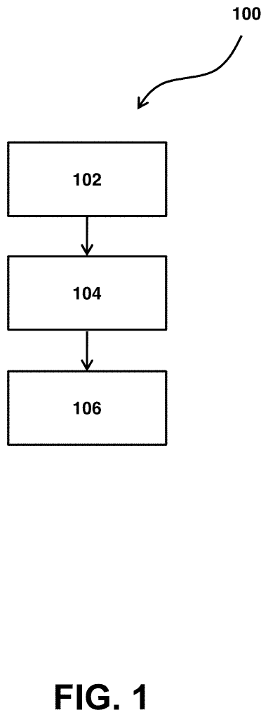

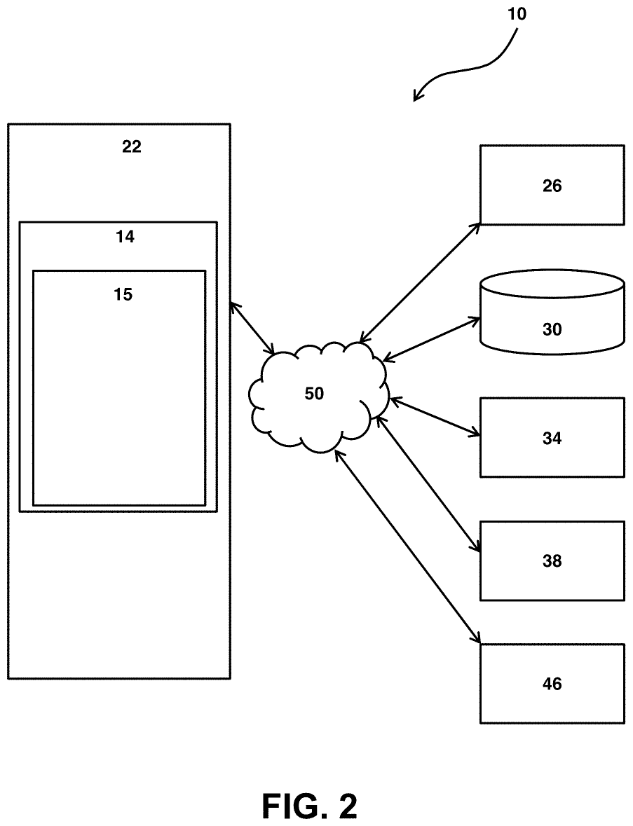

Accordingly, FIG. 1 illustrates a method 100 for real-time or near real time cellular drug-target engagement. Method 100 can be used for determining if a test compound can interact with a target polypeptide. Some or all of method 100 may be performed substantially in real time. Method 100 may be executed by a machine and/or a system such as machine 22 and/or system 10 illustrated in FIG. 2, and/or other machines and/or systems. Machine 22 and/or system 10 comprises one or more processors 14 configured by machine-readable instructions 15, and/or other components. The one or more processors 14 are configured to execute computer program components (formed by the machine readable instructions) that execute one or more operations of method 100. The operations of method 100 presented below are intended to be illustrative. In some embodiments, method 100 may be accomplished with one or more additional operations not described, and/or without one or more of the operations discussed. Additionally, the order in which the operations of method 100 are illustrated in FIG. 1 and described below is not intended to be limiting.

In some embodiments, method 100 may be implemented, at least in part, in one or more processing devices such as one or more processors 14 of machine 22 described herein (FIG. 2, e.g., a digital processor, an analog processor, a digital circuit designed to process information, an analog circuit designed to process information, a state machine, and/or other mechanisms for electronically processing information). The one or more processing devices may include one or more devices executing some or all of the operations of method 100 in response to instructions (e.g., machine readable instructions 15) stored electronically on an electronic storage medium (e.g., data store 30 of system 10). The one or more processing devices may include one or more devices configured through hardware, firmware, and/or software to be specifically designed for execution of one or more of the operations of method 100.

At an operation 102, a reaction solution is prepared. The reaction solution enables contact of a fusion protein within a reaction system. The reaction system comprises a test compound or vehicle, a denaturant, a nuclease acceptor, a nucleic acid substrate, a signal controller, and/or other components.

In some embodiments, the signal controller is any optional compound or excitant that may control the enzymatic activity of the complemented active enzyme, control the start of the target engagement reaction, control the speed of this reaction, and/or control the duration/maturity of the reaction. In some embodiments, the signal controller an antibody, chemical, peptide, UV, microwave, or light. In some embodiments, the signal controller is far-red light.

The fusion protein comprises: (a) the target polypeptide and nuclease acceptor domain, (b) the target polypeptide and a nuclease, or (c) the target polypeptide, an N-terminal domain of a nuclease and a first domain allowing for dimerization of the N-terminal domain to a C-terminal domain of the same nuclease fused to a second domain complementary to the domain allowing for dimerization. In some embodiments, the nuclease acceptor domain of (a) is an S-tag acceptor peptide, and wherein the nuclease is an S-protein. In some embodiments, the nuclease of (b) is ribonuclease. In some embodiments, the nuclease of (b) may be Cas9, Micrococcal nuclease, Rnase H, a non-natural nuclease hybrid such as Cas9-Fok1, or Cpf1/Cas12a. In some embodiments, the nuclease of (c) is a ribonuclease, the first domain allowing for dimerization is Coh2, and the second domain is DocS. In some embodiments, the nuclease of (c) is Cas9, the first domain allowing for dimerization is Coh2, and the second domain is DocS. In some embodiments, binding of the Coh2 and DocS domains is enabled by a signal controller, wherein the signal controller is far-red light.

In some embodiments, the signal controller is any optional compound or excitant that may control the enzymatic activity of the complemented active enzyme, control the start of the target engagement reaction, control the speed of this reaction, and/or control the duration/maturity of the reaction. In some embodiments, the signal controller an antibody, chemical, peptide, UV, microwave, or light. In some embodiments, the signal controller is far-red light. In some embodiments, the signal controller is far-red light.

In some embodiments, parts or the entirety of reaction solution is/are prepared outside or inside of the machine, such that: the entirety of reaction is prepared inside the machine; parts of reaction are prepared outside of the machine, then transferred into the machine as exemplified in Example 1 below; and/or certain reaction components are injected into the machine at once or sequentially.

In some embodiments, the solution is held within a container or a vessel compatible with real-time fluorescence measuring such as a tube or a multi-well plate compatible with real-time fluorescence measuring, and wherein the machine is programmed to read fluorescence signals from the reaction solution in real time. In some embodiments, the container is opaque to prevent light contamination from any other reaction. As described in Example 1 below, the solution is held within a black 96-well plate. In some embodiments, the multi-well plate comprises a microfluidic chip enabling reaction multiplexing. In some embodiments, a multiplex reaction comprises two or more different detectable labels. In some embodiments, a multiplex reaction comprises two or more different fluorescent labels.

At an operation 104, real time or near real time processing operations are performed. Operation 104 may be performed in real time or near real time by use of a machine (e.g., machine 22 shown in FIG. 2).

In some embodiments, the machine is a machine capable of measuring light, fluorescence, or derivative thereof. In some embodiments, the machine is a qPCR or a RT-qPCR machine. In some embodiments, the machine is a Fluidigm BioMark HD. In some embodiments, the machine is a BioRad CFX96/384 Real-Time PCR Machine. In some embodiments, the machine is a fluorescence plate reader such as a POLARStar® Omega 96-well plate reader, as exemplified in Example 1.

In some embodiments, the machine communicates in a circuit with other machines connected locally or remotely.

In some embodiments, the qPCR or RT-qPCR machine is programmed to mix, initiate, amplify signal, register signal, deconvolute data, analyze data, obtained from the reaction solution in real time. The program comprises any of: a) mixing reaction solution in batch wherein all ingredients are added from start of mixing, or by injection mode wherein ingredients added at various times; b) running reaction modules, each with their own commands, such that the data generated in one module can automatically define commands of the next module; c) running singular or multiplexed reactions; d) running kinetic series, where various combinations of temperatures and compound doses are tested without prior knowledge of target polypeptide's thermal profile; e) performing variously timed incubation or incubations with or without agitation or excitation; f) ramping temperature linearly, step-wise regularly, or irregularly, wherein each ramp step is defined with its own temperature range, speed, repeats and temperature/signal ratios; g) introducing alternating steps of temperature incubation, signal excitation, and pause; h) performing data registration, amplification, conversion, deconvolution, and/or analysis; i) communicating data within a local or remote machine circuit; j) parsing generated meta-data using programmed analysis methods to identify signal patterns; and/or other operations.

In some embodiments, the machine generates target engagement data configured to facilitate the multi-dimensional determination and quantification of engagement between the test compound and the target polypeptide. Multi-dimensional refers to a type of cell target engagement data that has several components, such as temperature range, dose range, fluorophore etc. and their various combinations, all in one setting.

In some embodiments, the target engagement data is configured to facilitate identification of binding stoichiometry, target occupancy, target competition, residence time, KD, K-on, K-off, and/or EC50. Example 1 below provides non-limiting examples of such engagement data and identification of such EC50 and KD.

At an operation 106, an amount and speed of a cleavage product of a nucleic acid substrate is detected. This may be detected in real time or near real time by use of a machine (e.g., machine 22 shown in FIG. 2) configured to detect fluorescence, generated light, or derivative thereof.

Provided herein, associated with or part of the method described above, in some embodiments, is a target-independent platform for monitoring drug-target engagement within cells. In some embodiments, the platform utilizes a modification of a cellular-thermal shift assay (CTSA) based on a premise that upon heating, a protein will begin to unfold, denature, and form insoluble aggregates as buried hydrophobic sites become exposed. The average, mean or absolute temperature at which protein melting and aggregation occurs is often described as a T_{agg} (or Tm). Heat-induced aggregation is sometimes altered by a small molecule that can indirectly interact with, or directly bind to a polypeptide causing a detectable shift in the T_{agg} (referred to as thermal shift). For some CTSA assays, the insoluble aggregated proteins are removed by centrifugation and soluble proteins that are stabilized by interaction or binding of a small molecule remain within the soluble fraction. An amount of the remaining soluble protein can be determined by various protein detection methods such as Western blotting. Such methods are often time consuming, expensive and tedious; require special training to conduct; and are therefore not amenable to high throughput drug screening methods. High throughput drug discovery requires a quick and inexpensive method that can be used to screen large numbers of chemical compounds in a relatively short amount of time to identify new drug candidates.

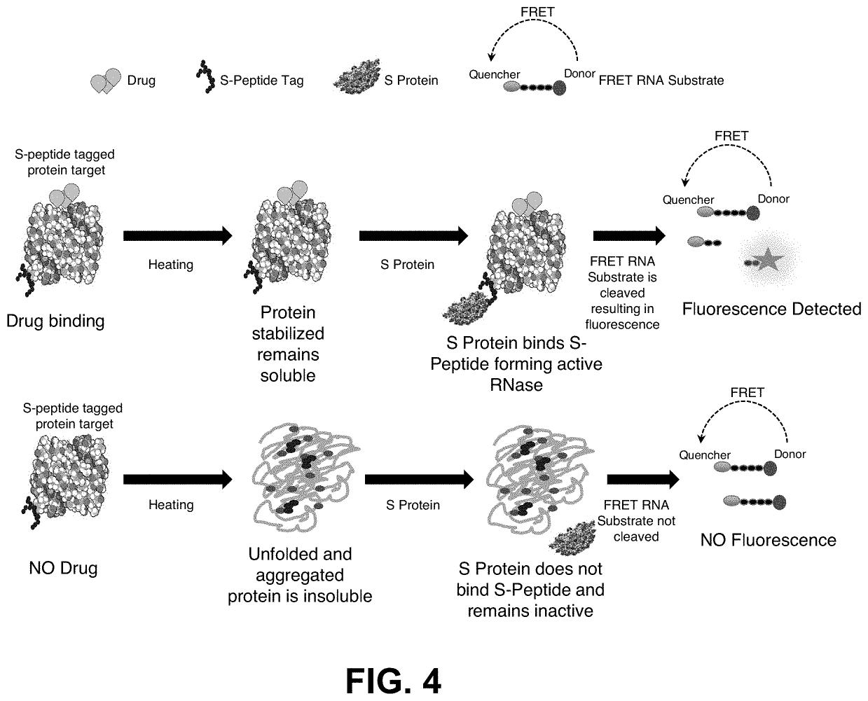

Provided herein is a modified enzyme complementation assay that can be used for high throughput screening of compounds that stabilize selected target polypeptides when exposed to heat (e.g., CTSA) or another denaturant (e.g., ultraviolet light, microwaves, radiation or chemical denaturants). The modified enzyme complementation assay utilized herein is based, in part, on assembly of (i) a ribonuclease (RNase) donor and (ii) an S-tag acceptor peptide that, when assembled, form a functional RNase enzyme complex that is capable of cleaving an RNA substrate (e.g., a FRET-labeled RNA substrate). The assay is monitored by detection of the presence or amount of the cleaved RNA substrate (FIG. 4), which provides a detectable signal when cleaved. The S-tag acceptor peptide is provided as a fusion protein comprising the S-tag acceptor peptide and a target polypeptide of interest. If the target polypeptide is denatured and aggregates due to the presence of a denaturant, the S-tag acceptor peptide portion of the aggregated fusion protein is unable to associate with the RNase acceptor. Therefore, when the target polypeptide denatures, an active RNase complex is not formed and no cleavage product is detected. If a test compound interacts with the target polypeptide and due to that interaction, prevents denaturation of the target polypeptide, a functional RNase enzyme complex is formed and cleavage of the RNA substrate is detected. Using this method, test compounds can be identified that interact with a target polypeptide of interest, such as a protein of a pathogen, to identify drug candidates, for example. The assay methods presented herein can be used as high-throughput platforms to screen a library of test compounds in a fast and efficient manner.

In some embodiments, the fusion protein of the assay can be expressed by a cell using a suitable method. The cell comprising the fusion protein can then be contacted with a test compound in the presence of a denaturant (e.g., heat) to determine if the test compound can prevent denaturation of the target polypeptide. There are many advantages of the assay methods presented herein compared to traditional screening methods. For example, (i) the assay methods herein are very rapid such that a cleaved RNA substrate can be detected with seconds to minutes, (ii) the S-tag acceptor peptide is relatively small and, as determined herein, does not interfere with denaturation of the larger target polypeptide portion of the fusion protein, (iii) the assay eliminates the need to perform Western blot analysis, (iv) the assay is relatively inexpensive, (v) the assay can be implemented as a multiplex assay to screen 100s or even thousands of test compounds, and (vi) the assay is amenable to automation.

Fusion Proteins

In some embodiments, the method comprises contacting a fusion protein with one or more of a test compound, a denaturant, a nuclease donor, and a nucleic acid substrate. In some embodiments, the method comprises contacting a fusion protein with one or more of a test compound, a denaturant, an RNase donor and a nucleic acid substrate flanked by a pair of FRET labels (e.g., a FRET-labeled RNA substrate). In certain embodiments, a fusion protein comprises a target polypeptide and a nuclease acceptor. In some embodiments, the nuclease acceptor is an S-tag acceptor domain. In some embodiments, a fusion protein comprises a target polypeptide and an S-tag acceptor peptide.

A fusion protein may comprise one or more or two or more nuclease acceptors. In some embodiments, the fusion protein may comprise one or more or two or more S-tags. Any suitable target polypeptide of interest can be used for a method herein. A target polypeptide and nuclease acceptor (e.g., an S-tag acceptor peptide) can be attached by a suitable covalent bond or linker. Non-limiting examples of linkers include one or more amino acids, peptide linkers, alkanes. PEG, an optionally substituted C1-C50 alkyl, optionally substituted C2-C50 alkenyl, alkynyl, acyl, acyloxy, alkoxy, aryloxy, cycloalkyl, cycloalkenyl, cycloalkoxy, aryl, aminocarbonyl, azido, carboxy, silanes, thiols, sulfoxide, sulfones, sulfonate ester, cyano, amide, amino, ester, phosphonic acid, other suitable polymers, derivatives thereof, the like and combinations thereof. In some embodiments, a linker comprises a peptide comprising two or more amino acids. 2 to 100 amino acids, 5 to 100 amino acids, 2 to 50 amino acids, 5 to 50 amino acids, 2 to 25 amino acids, 5 to 25 amino acids, 2 to 20 amino acids, 5 to 20 amino acids, 2 to 10 amino acids or 5 to 10 amino acids. In some embodiments, a linker comprises a peptide of 1 to 20, 1 to 10, or 1 to 5 amino acids. In some embodiments, a fusion protein is assembled by attaching a target polypeptide to a nuclease acceptor (e.g., an S-tag acceptor peptide), for example by use of a suitable linking chemistry. In some embodiments, a fusion protein is expressed using a suitable expression system, as a single contiguous polypeptide comprising the a nuclease acceptor (e.g., an S-tag acceptor peptide) and the target polypeptide. In some embodiments, a target polypeptide is attached to the C-terminus of a nuclease acceptor (e.g., an S-tag acceptor peptide). In some embodiments, a target polypeptide is attached to the N-terminus of a nuclease acceptor (e.g., an S-tag acceptor peptide).

In some embodiments, the fusion protein comprises a target polypeptide and a nuclease acceptor. In some embodiments, the fusion protein comprises a target polypeptide and a nuclease. In some embodiments, the fusion protein comprises a target polypeptide, an N-terminal domain of a nuclease and a first domain allowing for dimerization of the N-terminal domain to a C-terminal domain of the same nuclease fused to a second domain complementary to the domain allowing for dimerization. In some embodiments, the nuclease acceptor is an S-tag donor peptide and the nuclease is an S protein. In some embodiments, a split-Cas9 system is designed where the nuclease domain and the helical domain are cloned and expressed independently, then complemented in a controlled reaction. This enables a more controlled nuclease activity of the Cas9 enzyme. Either of these domains or their sub-domains, can be fused with a target polypeptide, then complemented with the rest of the enzyme in a cellular target engagement system (Wright A V et al. “Rational design of a split-Cas9 enzyme complex.” Proceedings of the National Academy of Sciences of the United States of America vol. 112, 10 (2015): 2984-9. doi:10.1073/pnas.1501698112). In some embodiments, the nuclease is selected from the group consisting of Cas9, Micrococcal nuclease, Rnase H, a non-natural nuclease hybrid such as Cas9-Fok1, and Cpf1/Cas12a. In some embodiments, the nuclease is Cas9, the first domain allowing for dimerization is Coh2, and the second domain is DocS. Coh2 and DocS are two C. thermocellum proteins that interact with high affinity. A Coh2-DocS complementation system can be designed where either of these proteins, or domains or sub-domains thereof, is fused with a target polypeptide, then complemented with the rest of the Coh2-DocS complex in a cellular target engagement system (Yu Y et al. Engineering a far-red light-activated split-Cas9 system for remote-controlled genome editing of internal organs and tumors. Sci Adv. 2020; 6(28):eabb1777. Published 2020 Jul. 10. doi:10.1126/sciadv.abb1777).

In some embodiments, the signal controller is any optional compound or excitant that may control the enzymatic activity of the complemented active enzyme, control the start of the target engagement reaction, control the speed of this reaction, and/or control the duration/maturity of the reaction. In some embodiments, the signal controller is an antibody, chemical, peptide, temperature, UV, microwave, or light. In some embodiments, the signal controller is far-red light. In some embodiments, binding of the Coh2 and DocS domains is enabled by a signal controller, wherein the signal controller is far-red light.

A fusion protein may comprise one or more linkers. In some embodiments, a fusion protein comprises one or more linkers between a target polypeptide and a nuclease acceptor domain. In some embodiments, a fusion protein comprises one or more linkers between a target polypeptide and a nuclease. In some embodiments, a fusion protein comprises one or more linkers between a target polypeptide and an N-terminal domain of a nuclease and a first domain allowing for dimerization of the N-terminal domain to a C-terminal domain of the same nuclease fused to a second domain complementary to the domain allowing for dimerization.

Nucleases

The methods herein rely, in part, on the assembly of a functional nuclease enzyme complex wherein an N-terminal domain of a first nuclease enzyme and a C-terminal domain of a second nuclease enzyme assemble to form an active enzyme complex capable of cleaving a nucleic acid substrate. The methods herein rely, in part, on the assembly of a functional RNase enzyme complex wherein an N-terminal domain of a first RNase enzyme and a C-terminal domain of a second RNase enzyme assemble to form an active enzyme complex capable of cleaving a FRET-labeled RNA or DNA/RNA substrate. In some embodiments, a suitable RNase has a domain structure such that (i) an N-terminal portion of the protein can be separated from a C-terminal portion of the protein, (ii) the isolated N-terminal and C-terminal portions are devoid of enzymatic activity, and (iii) the isolated N-terminal portion and isolated C-terminal portion of the RNase can self-assemble in a non-covalent manner to form a functional RNase enzyme. Non-limiting examples of a suitable RNase protein that can be used for a method herein includes bovine RNase A (accession AAB35594; UniprotKB P61823)); human RNase A (NCBI accession NP_002924.1); chimpanzee RNase A (NCBI accession XP_520673.1); canine RNase A (NCBI accession number XP_532618.2); mouse RNase A (NCBI accession number NP_035401.2); rat RNase A (NCBI accession number XP_223969.2); homologues thereof, the like, and derivatives thereof having RNase activity. In some embodiments, an RNase is bovine pancreatic RNase A (e.g., UniProtKB P61823) or derivative thereof, having the following sequence of the mature protein, KETAAAKFERQHMDSSTSAASSSNYCNQMMKSRNLTKDRCKPVNTFVHESLADVQAV CSQKNVACKNGQTNCYQSYSTMSITDCRETGSSKYPNCAYKTTQANKHIIVACEGNPY VPVHFDASV (SEQ ID NO:21), where the underlined portion represents the S-tag peptide sequence of the protein.

In some embodiments, the nuclease is a ribonuclease or a deoxyribonuclease. In some embodiments, the nuclease is a ribonuclease. In some embodiments, the nuclease is a deoxyribonuclease.

In some embodiments, the nuclease is a sequence-specific nuclease. In some embodiments, the nuclease is a Clustered Regularly Interspaced Short Palindromic Repeats (CRISPR)-associated protein (i.e., a Cas protein). In some embodiments, the Cas protein is Cas9 (Csn1), Cas12a (Cpf1), Cas12b (C2c1), Cas13a (C2c2), Cas13b (C2c6) or Cas13c (C2c7). In some embodiments, the Cas protein is Cas9.

In some embodiments, the nuclease is a non-natural nuclease hybrid. In some embodiments, the non-natural nuclease hybrid is Cas9-Fok1.

In some embodiments, the nuclease is an RNase. In some embodiments, the RNase is an RNase A, RNase H, or RNase S.

In some embodiments, the nuclease is a Micrococcal nuclease.

RNase Donor

In some embodiments, an RNase donor comprises or consists of a C-terminal portion of a suitable RNase. In some embodiments, an RNase donor is an RNase S protein. An RNase donor (e.g., an RNase S protein) can be made using a suitable method. In one non-limiting example, an RNase donor is made by treating an RNase A with subtilisin, which, under appropriate conditions, cleaves a single peptide bond of an RNase thereby providing an N-terminal portion (i.e., the S peptide, e.g., about 15-25 amino acids) and a C-terminal portion (i.e., the S protein, e.g., about 90 to 120 amino acids). In another, non-limiting, example an RNase donor is made using recombinant technology such that the C-terminal (S-protein) portion of an RNase is expressed using a suitable expression system. An isolated RNase donor is substantially devoid of enzymatic activity (e.g., RNase activity) until it contacts a suitable S-tag acceptor peptide.

In some embodiments, an RNase donor comprises an amino acid sequence having at least 80%, at least 85%, at least 90%, at least 91%, at least 92%, at least 93%, at least 94%, at least 95%, or 100% identity to the amino acid sequence MSSSNYCNQMMKSRNLTKDRCKPVNTFVHESLADVQAVCSQKNVACKNGQTNCYQS YSTMSITDCRETGSSKYPNCAYKTTQANKHIIVACEGNPYVPVHFDASV (SEQ ID NO:19) or MSSSNYCNQMMKSRNLTKDRCKPVNTFVHESLADVQAVCSQKNVACKNGQTNCYQS YSTMSITDCRETGSSKYPNCAYKTTQANKHIIVACEGNPYVPVHFD (SEQ ID NO:20). In some embodiments, an RNase donor comprises a polypeptide comprising at least 85, at least 90, at least 95, or at least 100 contiguous amino acids of the amino acid sequence of SEQ ID NO:19 or SEQ ID NO:20. Derivatives of an RNase donor may comprise conservative amino acid substitutions or amino acid analogues. In some embodiments, a RNase donor comprises a suitable amino acid tag (e.g., a histidine tag, a flag tag, or the like).

Nuclease Acceptor

In some embodiments, a nuclease acceptor, or a nuclease acceptor domain, is a domain allowing for assembly of a functional nuclease enzyme complex wherein an N-terminal domain of a first nuclease and a C-terminal domain of a second, or the same first nuclease assemble to form an active enzyme complex capable of cleaving a nucleic acid substrate. In some embodiments, a nuclease acceptor is an acceptor peptide that comprises a suitable S-tag. A nuclease acceptor peptide often comprises or consists of a relatively small N-terminal portion of a nuclease protein. An S-tag acceptor peptide often comprises or consists of a relatively small N-terminal portion of an RNase protein. An S-tag peptide confers RNase enzymatic activity when it non-covalently associates with an RNase donor (e.g., an S Protein). Any suitable S-tag acceptor peptide and RNase donor combination can be used for a method herein. Various combinations of an S-tag acceptor peptide and an RNase donor can be readily tested for use in a method herein without requiring undue experimentation. Often an S-tag acceptor peptide derived from one species will associate with an RNase donor derived another species to form a functional enzyme complex. In some embodiments, derivatives of an S-tag peptide and/or derivatives of an RNase donor can be used for a method herein.

In some embodiments, the length of a nuclease acceptor peptide is in a range of 10 to 60 amino acids, 10 to 40 amino acids, 15 to 30 amino acids, 15 to 25 amino acids, 10 to 25 amino acids, or 8 to 25 amino acids. In some embodiments, a nuclease acceptor peptide comprises, consists of, or consists essentially of about 15 to 25 amino acids. In certain embodiments, a nuclease acceptor peptide has no detectable secondary structure. In certain embodiments, a nuclease acceptor peptide is highly soluble. In certain embodiments, a nuclease acceptor peptide has no net charge a neutral pH.

In some embodiments, the length of an S-tag acceptor peptide is in a range of 10 to 60 amino acids, 10 to 40 amino acids, 15 to 30 amino acids, 15 to 25 amino acids, 10 to 25 amino acids, or 8 to 25 amino acids. In some embodiments, an S-tag acceptor peptide comprises, consists of, or consists essentially of about 15 to 25 amino acids. In certain embodiments, an S-tag acceptor peptide has no detectable secondary structure. In certain embodiments, an S-tag acceptor peptide is highly soluble. In certain embodiments, an S-tag acceptor peptide has no net charge a neutral pH.

In some embodiments, an S-tag acceptor peptide comprises or consists of a peptide having an amino acid sequence having at least 80%, at least 85%, at least 90%, at least 91%, at least 92%, at least 93%, at least 94%, at least 95%, or 100% identity to an amino acid sequence selected from KETAAAKFERQHMDSSTSAA (SEQ ID NO:1), KETNWAWFWDQHMDSSTSA (SEQ ID NO:2), KETGWALFVQQHMDSSTSA (SEQ ID NO:3), KETVMANFQMQHMDSSTSA (SEQ ID NO:4), KETGDAVFARQHMDSSTSA (SEQ ID NO:5), KETGWAAFVKQHMDSSTSA (SEQ ID NO:6), KETGWATFVEQHMDSSTSA (SEQ ID NO:7), KETKLAFFLKQHMDSSTSA (SEQ ID NO:8), KETWWAWFFGQHMDSSTSA (SEQ ID NO:9); KETTWAEFTWQHMDSSTSA (SEQ ID NO:10), KETPWASFNKQHMDSSTSA (SEQ ID NO: 11), KETAMAMFVTQHMDSSTSA (SEQ ID NO:12), KETLWAWFMWQHMDSSTSA (SEQ ID NO:13), KETAAAKFERQHMDS (SEQ ID NO:14), KETAAAKFERQHMNS (SEQ ID NO:15), NRAWSEFLWQHLAPV (SEQ ID NO:16), NRGWSEFLWQHHAPV (SEQ ID NO:17) and NRAWSVFQWQHIAPA (SEQ ID NO:18), and derivatives thereof. In some embodiments, an S-tag acceptor peptide comprises a at least 10, at least 11, at least 12, at least 13, at least 14, or at least 15 contiguous amino acids of an amino acid sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, and SEQ ID NO:18, and derivatives thereof. In some embodiments an S-tag acceptor peptide is an S-peptide discloses in Backer et al. (2002) Protein Expression and Purification 26:455-461; Dwyer et al. (2001) Biochemistry 40(45):13491-500; Kim and Raines (1993) Protein Science 2:348-356; and Beintema, J. J. (1987) Life Chem. Rep. 4:333-389, which are incorporated herein by reference. Derivatives of S-tag acceptor peptides may comprise conservative amino acid substitutions or amino acid analogues. In certain embodiments, S-tag acceptor peptides can be made and/or selected for using methods described in Yu and Smith (1996) Methods in Enzymology 267, 3-27; and Goldberg et al. (1999) PNAS 96:2019-2024.

Target Polypeptides

A fusion protein may comprise a suitable target polypeptide of interest. In some embodiments, a target polypeptide has a length in a range of 10 to 1000, 10 to 500, 10 to 250, 10 to 125 or 10 to 50 amino acids. In certain embodiments, a target polypeptide comprises a polypeptide, or portion thereof, derived from a suitable pathogen, non-limiting examples of which include a virus, a bacteria, a fungus, and a parasite. Non-limiting examples of a virus include a virus of the family Adenoviridae, Papovaviridae, Parvoviridae, Herpesviridae, Poxviridae, Anelloviridae, Pleolipoviridae, Reoviridae, Picornaviridae, Caliciviridae, Togaviridae, Arenaviridae, Flaviviridae, Orthomyxoviridae (e.g., Influenzavirus), Paramyxoviridae, Bunyaviridae, Rhabdoviridae, Filoviridae, Coronaviridae (e.g., SARS, SARS-CoV-2, MERS, HKU1), Astroviridae, Bornaviridae, Arteriviridae, Rotavirus and Hepeviridae. In certain embodiments, the virus is SARS-Cov2 coronavirus. In certain embodiments, the virus an influenza virus. In certain embodiments, the virus a Hepatitis A, B or C virus. In certain embodiments, the virus a Herpes virus. In some embodiments, the pathogen is a bacteria. In certain embodiments the bacteria is Helicobacter pylori, Mycobacterium tuberculosis or a Mycobacterium.

In some embodiments, a target polypeptide is modified. Non-limiting examples of a modification of a polypeptide include one or more amino acid substitutions, deletions or additions. For example, in some embodiments, a method herein is conducted as a multiplex or high-throughput assay using a plurality of vessels (e.g., microtiter wells), where each well comprises a different fusion protein, each fusion protein comprising a different target polypeptide. The different target polypeptides can be different proteins and/or modification of a target polypeptide. In one non-limiting example, the target polypeptide is a viral capsid protein (e.g., a spike protein of SARS-Cov2, or a haemagluttinin protein of influenza virus), and each of the vessels or microtiter wells includes a different modification of the viral capsid protein (e.g., random or computer-generated mutations).

In some embodiments, a target polypeptide is a naturally occurring polypeptide, or a portion thereof. In some embodiments, a target polypeptide is synthetic. In some embodiments, a target polypeptide is naturally produced or recombinantly produced. In some embodiments, a target polypeptide comprises a protein, or a portion of a protein, that is isolated, purified and/or recombinantly expressed as a soluble protein (e.g., isolated, purified or expressed as a soluble fusion protein). In some embodiments, a target polypeptide or fusion protein is expressed in or on a cell. In some embodiments, a target polypeptide or fusion protein is expressed on a surface of a cell. In certain embodiments, the cell is a suitable eukaryotic cell (e.g., a mammalian cell). In certain embodiments, the cell is a prokaryotic cell (e.g., a bacteria).

In some embodiments, the target polypeptide is MTH1. In some embodiments, the nuclease acceptor is an S-tag peptide. In some embodiments, there no linker between MTH1 and the S-tag peptide. In some embodiments, the amino acid sequence of a MTH1-S-tag construct is shown in SEQ ID NO:25.

Test Compounds

The methods herein can be used to screen any suitable test compound or library of test compounds.

As used herein, the phrase “test compound” refers to any suitable compound that can be screened for the ability to interact with, or bind to, a target polypeptide of interest using a method described herein. Non-limiting examples of a test compound include small compounds (e.g., small organic or inorganic molecules), large compounds (e.g., greater than 5000 Da), polysaccharides, carbohydrates, sugars, fatty acids, lipids, biological macromolecules, (e.g., peptides, polypeptides, proteins, peptide analogs and derivatives, peptidomimetics, nucleic acids, nucleotides, nucleotide analogs), naturally occurring or synthetic compounds, binding agents (e.g., antibodies, or binding fragments thereof, including non-naturally occurring and synthetic binding agents (e.g., TandAbs, nanobodies, aptamers, BiTEs, SMIPs, DARPins, DNLs, affibodies, Duocalins, adnectins, fynomers, Kunitz Domains Albu-dabs, DARTs, DVD-IG, Covx-bodies, peptibodies, scFv-Igs, SVD-Igs, dAb-Igs, Knob-in-Holes, triomAbs, and the like), derivatives thereof, polymers thereof, salts thereof, isomers thereof, polymorphs thereof, and combinations thereof. In some embodiments, a test compound is contained within an extract made from biological materials such as extracts of bacteria, plants, fungi, animal cells, or animal tissues. In some embodiments, a test compound is contained within a biological fluid. Accordingly, in some embodiments, a test compound comprises an extract or biological fluid. Small compounds include molecules having a molecular weight greater than about 40 daltons (Da), but less than 5000 Da, less than 3000 Da, or less than 1000 Da. Small compounds may comprise any suitable chemical moiety or group, non-limiting examples of which include alkanes, alkenes, alkynes, alcohols, halogens, ketones, aldehydes, carboxylic acids, ethers, esters, amines, amides, saturated, partially saturated or unsaturated ring structures, nucleotides, nucleosides, polyatomic nonmetals (e.g., P, S, Se), transition metals, post-transition metals, metalloids, the like, salts thereof, and combinations thereof.

In certain embodiments, test compounds include synthetic or naturally occurring compounds of a suitable library. A multitude of small molecule libraries are known in the art, some of which are commercially available. Commercially available compound libraries can be obtained from, for example, ArQule, Pharmacopia, graffinity, Panvera, Vitas-M Lab, Biomol International and Oxford. Methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156 (2001). Chemical compound libraries from, for example, NIH Roadmap, Molecular Libraries Screening Centers Network (MLSCN) can also be used. Any suitable method can be used to make a small compound library. A compound library can be screened using a suitable method described herein.

In certain embodiments, a test compound comprises a molecular weight of 40 to 500,000 Da, 40 to 200,000 Da, 40 to 100,000 Da, 40 to 50,000 Da, 40 to 25,000 Da, 40 to 10,000 Da, 40 to 5000 Da, or 40 to 1000 Da. In certain embodiments, a test compound comprises a molecular weight of 5000 to 500,000 Da, 10,000 to 500,000 Da, 25,000 to 500,000 Da, or 5000 to 100,000 Da.

A test compound can be tested at any suitable concentration. In some embodiments, a test compound is tested at a concentration of at least 1 pM, at least 10 pM, at least 100 pm, at least 1 nM, at least 10 nM, at least 100 nM, at least 1 μM, at least 10 μM, at least 100 μM or at least 1 mM. In some embodiments, a test compound is tested at a concentration in a range of 1 pM to 100 mM, 1 pM to 10 mM, 1 pM to 1 mM, 10 pM to 100 mM, 10 pM to 10 mM, 10 pM to 1 mM, 100 pM to 100 mM, 100 pM to 10 mM, or 100 pM to 1 mM. In some embodiments, a test compound is tested at a concentration of less than 100 mM, less than 10 mM, less than 1 mM or less than 100 nM. In some embodiments, a test compound is tested or assayed at one or more different concentrations.

Substrates

In some embodiments a fusion protein and/or an RNase donor, or a mixture or cell comprising a fusion protein and an RNase donor is contacted with suitable nucleic acid substrate. In some embodiments, a nucleic acid substrate is a nucleic acid that can be cleaved by an RNase disclosed herein or by an assembled RNase enzyme complex described herein (e.g., comprising an S-tag acceptor peptide and RNase acceptor). In some embodiments, a nucleic acid substrate comprises RNA and/or DNA. In some embodiments, a nucleic acid substrate comprises ribonucleotides, deoxyribonucleotides, analogues thereof or mixtures thereof. A nucleic acid substrate may be single stranded or double stranded. In certain embodiments, a nucleic acid substrate comprises 2 or more. 3 or more. 5 or more or 10 or more nucleotides. In certain embodiments, a nucleic acid substrate comprises at least 1 pyrimidine nucleotide. In certain embodiments, a nucleic acid substrate comprises at least 2, at least 3 or at least 4 adjacent pyrimidine nucleotides.

In certain embodiments, a nucleic acid substrate comprises a suitable detectable label. In certain embodiments, a detectable label of a substrate provides a detectable signal, a change in a detectable signal (e.g., an increase or decrease in a signal, or a wavelength shift), or loss of a detectable signal upon cleavage of a labeled substrate by an RNase. In some embodiments, a detectable signal emitted from a label of a nucleic acid substrate is undetectable until after cleavage of the substrate. In some embodiments, a detectable signal emitted from a label of a nucleic acid substrate is enhanced after cleavage of the substrate. In certain embodiments, a detectable signal emitted from a label of a nucleic acid substrate is reduced after cleavage of the substrate.

Non-limiting examples of a detectable label include a metallic label, a fluorescent label, a fluorescent protein (e.g., green fluorescent protein (GFP)), a PH sensitive protein or PH sensitive GFP (e.g., a PHlourin, or the like), any suitable fluorophore (e.g., mCherry), a chromophore, a chemiluminescent label, an electro-chemiluminescent label (e.g., Origen™), a phosphorescent label, a quencher (e.g., a fluorophore quencher), a fluorescence resonance energy transfer (FRET) pair (e.g., donor and acceptor), a protein (e.g., an enzyme (e.g., horseradish peroxidase, β-galactosidase, luciferase, alkaline phosphatase and the like)), an antigen or part thereof, a linker, a member of a binding pair), an enzyme substrate, a small molecule (e.g., biotin, avidin), a mass tag, quantum dots, nanoparticles, the like or combinations thereof. Any suitable fluorophore or light emitting material can be used as a detectable label. Non-limiting examples of a fluorescent label include fluorescein, rhodamine, Texas Red, phycoerythrin, allophycocyanin, 6-carboxyfluorescein (6-FAM), 2,7-dimethoxy-4′,5′-dichloro-6-carboxyfluorescein (JOE), 6-carboxy-X-rhodamine (ROX), 6-carboxy-2′,4′,7,4,7-hexachlorofluorescein (HEX), 5-carboxyfluorescein (5-FAM) or N.N.N′,N′-tetramethyl-6-carboxyrhodamine (TAMRA), cyanine dyes, such as Cy3, Cy5, Alexa 542, Bodipy 630/650, fluorescent particles, fluorescent semiconductor nanocrystals, the like, and combinations thereof. A detectable label can be detected and/or quantitated by a variety of suitable techniques such as, for example, digital photography, flow cytometry, gel electrophoresis, chip analysis (e.g., any chip methodology), microarray, mass spectrometry, cytofluorimetric analysis, fluorescence microscopy, confocal laser scanning microscopy, laser scanning cytometry, suitable plate readers, the like and combinations thereof.

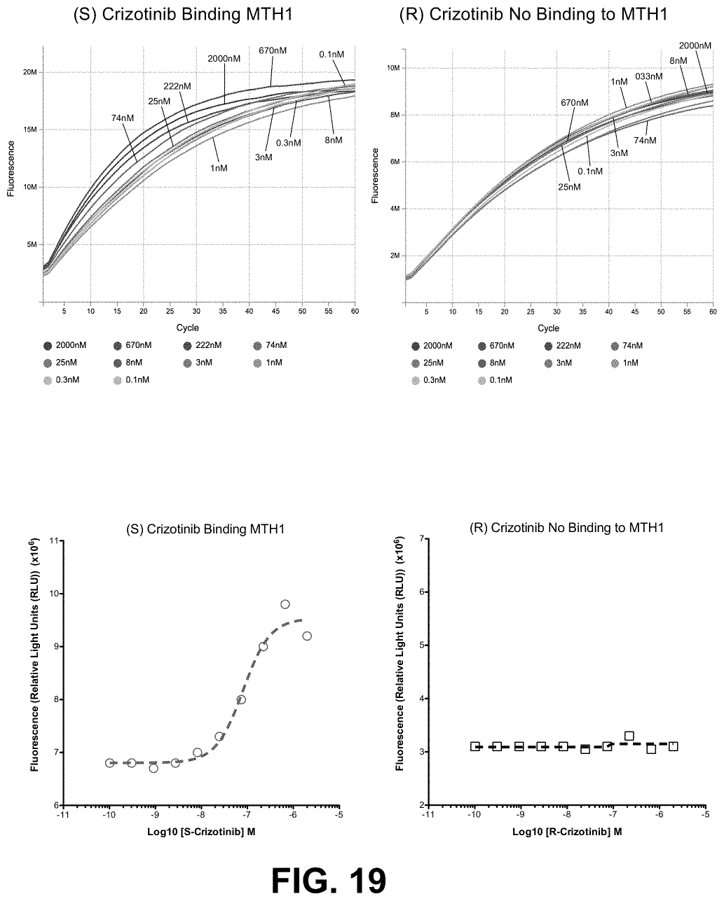

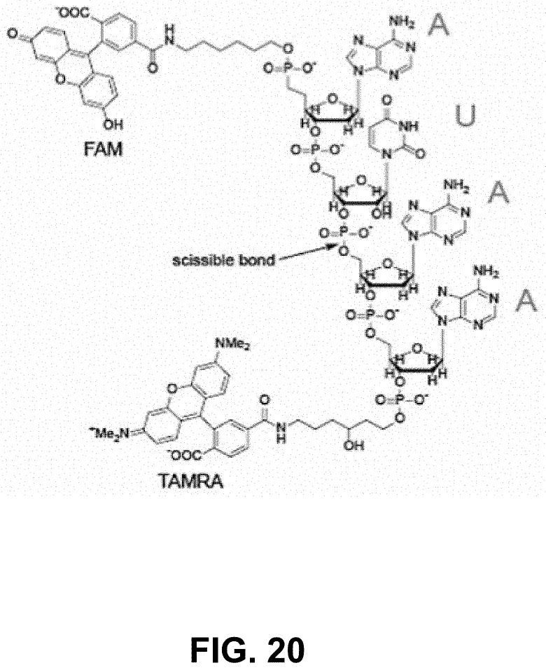

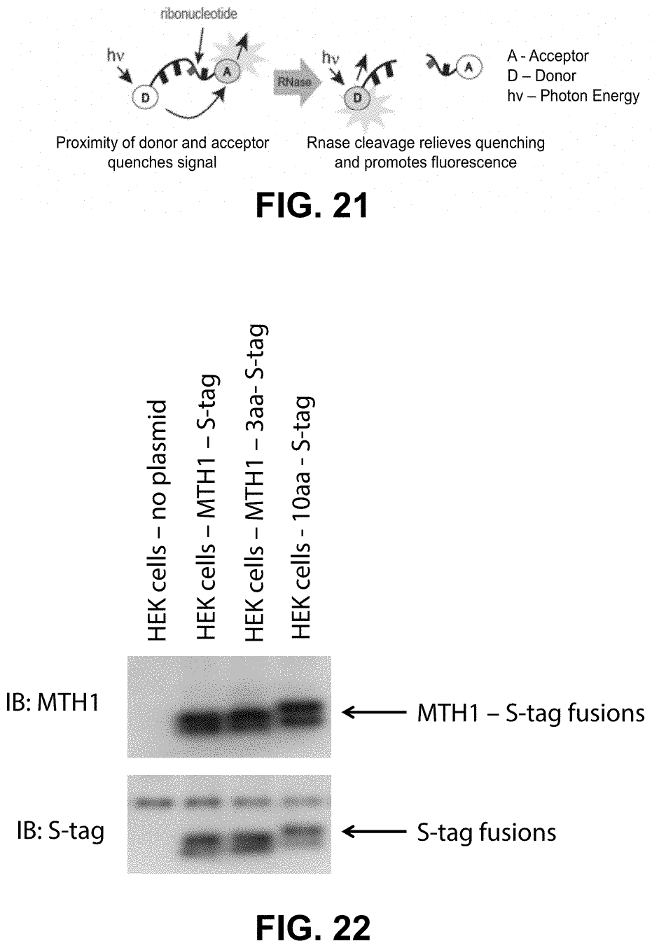

In some embodiments, a nucleic acid substrate comprises a suitable fluorescence energy transfer (FRET) label. One advantage of FRET-labeled nucleic acids is that they can readily enter an intact cell thereby allowing detection of cleavage of the substrate in whole cells. In certain embodiments, a nucleic acid substrate comprises a pair of FRET labels comprising a suitable fluorescent donor/acceptor pair separated by polynucleotide comprising an RNase cleavable sequence such that fluorescence emission of the donor is quenched until the substrate is cleaved by an assembled RNase complex. In certain embodiments, a nucleic acid substrate comprises a FRET label comprising a suitable fluorescent donor/acceptor pair separated by polynucleotide comprising an RNase cleavable sequence such that fluorescence emission of the donor is quenched until the substrate is cleaved by an assembled RNase complex. In certain embodiments a FRET label comprises 6-carboxyfluorescein (6-FAM) and 6-carboxy-tetramethylrhodamine (6-TAMRA)(FIG. 19) separated by two or more contiguous nucleotides. In some embodiments a nucleic acid substrate comprises (6-FAM)-X-(6-TAMRA), wherein X comprises a polynucleotide comprising 2 to 10 nucleotides. In some embodiments a nucleic acid substrate comprises 6-FAM-dArUdAdA-6-TAMRA (FIG. 19), where rU is uridine and dA is deoxyadenine.

In certain embodiments of a method described herein, an amount of cleavage of a substrate is determined using a suitable method. In certain embodiments the presence or absence of a cleavage product of a nucleic acid substrate is determined using a suitable method. In some embodiments, the presence, absence or amount of cleavage of a pair of FRET labels labeled nucleic acid substrate is determined using a suitable method. The presence, absence or amount of cleavage of a labeled nucleic acid substrate can be determined at a suitable time after contacting a cell or mixture with a nucleic acid substrate or nuclease donor. The presence, absence or amount of cleavage of a labeled nucleic acid substrate can be determined at a suitable time after contacting a cell or mixture with a nucleic acid substrate or RNase donor. In some embodiments, the presence, absence or amount of cleavage of a labeled nucleic acid substrate is determined dynamically over a period of time, for example to determine a rate of cleavage. A predetermined amount of a cleavage product can be determined using a suitable positive control. For example, a positive control may utilize a fusion protein comprising a known protein, and a test compound that is known to interact with and stabilize the known protein when exposed to a denaturant, thereby allowing complementation of the S-tag acceptor peptide with an RNase donor protein to form an active nuclease complex. The presence or amount of nucleic acid substrate cleaved by the active nuclease complex of the positive control can be used as a base line to detect other test compounds that interact with a target polypeptide.

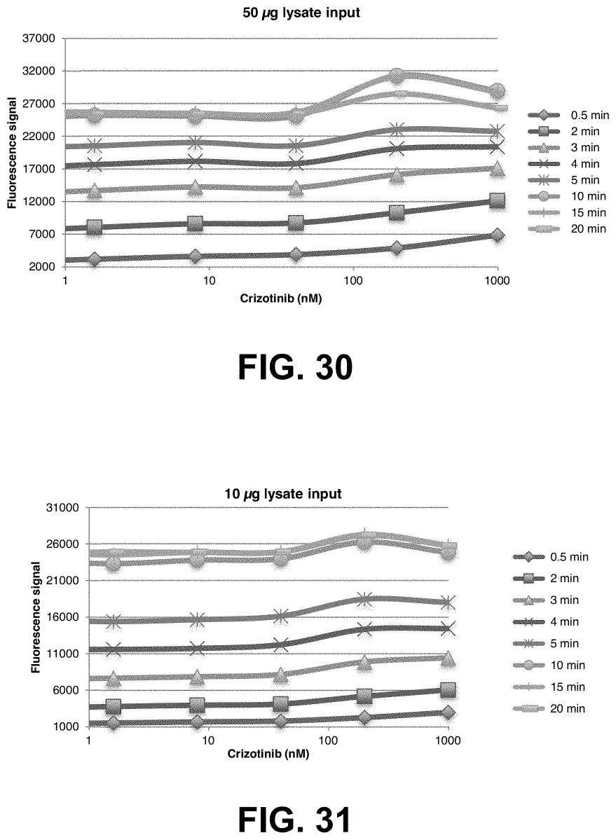

FRET-labeled Cleavage and detection of cleavage of a FRET-labeled nucleic acid substrate is very rapid (often requiring less than 30 seconds) (e.g., see FIGS. 29-31). Accordingly, in certain embodiments, FRET-labeled substrates are used for high-throughput methods described herein and allow for automation of the methods described herein.

Denaturants

In some embodiments, a fusion protein is contacted with a denaturant. Non-limiting examples of a denaturant include (i) heat, (ii) ultraviolet light, (iii) microwaves, (iv) radiation and (iv) a chemical denaturant.

In certain embodiments, contacting a fusion protein with a denaturant comprises contacting a fusion protein, or a cell or mixture comprising a fusion protein, with heat. In some embodiments, a fusion protein is contacted with an amount of heat sufficient to denature and/or aggregate a fusion protein. In certain embodiments, contacting a fusion protein with heat comprises heating a fusion protein, or a cell or mixture comprising a fusion protein, to a temperature in a range of 40° C. and 90° C., 40° C. and 80° C., 40° C. and 75° C., 45° C. and 75° C., 50° C. and 75° C., or 55° C. and 70° C. In certain embodiments, contacting a fusion protein with heat comprises heating a fusion protein, or a cell or mixture comprising a fusion protein, to a temperature of at least 40° C., at least 50° C., at least 60° C., at least 65° C., or at least 70° C. In certain embodiments, contacting a fusion protein with heat comprises heating a fusion protein, or a cell or mixture comprising a fusion protein, from a temperature of about 30° C.-40° C. to a temperature of about 50° C. to 70° C. In certain embodiments, contacting a fusion protein with heat comprises exposing a fusion protein, or a cell or mixture comprising a fusion protein, to a temperature gradient in a range of 30° C. to 90° C., 30° C. to 80° C., 30° C. to 75° C., 37° C. to 75° C., 37° C. to 70° C. In some embodiments, contacting a fusion protein with a temperature gradient comprises increasing the temperature of a fusion protein, or a cell or mixture comprising a fusion protein, at a rate of at least about 1 to 10° C. per minute, or about 1 to 5° C. per minute. In some embodiments, contacting a fusion protein with heat comprises exposing a fusion protein, or a cell or mixture comprising a fusion protein, to heat, or an increasing temperature gradient for a period of time of at least 30 seconds, at least 1 minute, at least 3 minutes or at least 5 minutes.

In certain embodiments, a fusion protein is contacted with, or exposed to, a denaturant for period of time. In some embodiments, a fusion protein is contacted with, or exposed to a denaturant for a time period of at least 20 seconds, at least 30 seconds, at least 1 minute, at least 3 minutes or at least 5 minutes.

In certain embodiments, contacting a fusion protein with a denaturant comprises subjecting a fusion protein, or a cell or mixture comprising a fusion protein, to one or more freeze-thaw cycles.

In certain embodiments, contacting a fusion protein with a denaturant comprises exposing a fusion protein, or a cell or mixture comprising a fusion protein, with a suitable amount of electromagnetic radiation sufficient to denature a protein, non-limiting examples of which include ultraviolet light, microwaves or radiation (e.g., beta or gamma radiation). In some embodiments, denaturation of a protein can be performed with UV at 250 nm for 5 min, or 0.1 joules/cm².

In certain embodiments, contacting a fusion protein with a denaturant comprises contacting a fusion protein, or a cell or mixture comprising a fusion protein, with a chemical denaturant, non-limiting examples of which include an oxidizing agent, a toxin, an acid, a base, a carcinogen, and a chemotherapeutic agent. Simple routine test can be performed to quickly determine the amount of a chemical denaturant needed to denature a fusion protein. In some embodiments, a fusion protein, or a cell or mixture comprising a fusion protein, is contacted with a chemical denaturant in the presence of a test compound, and the chemical denaturant is substantially removed before contacting the fusion protein with an RNase donor protein (e.g., by incorporating a washing step). In certain embodiments, contacting a fusion protein with a denaturant comprises contacting a fusion protein, or a cell or mixture comprising a fusion protein, with a chemical denaturant at a concentration in a range of 1 fM to 500 mM, 1 pM to 100 mM, 1 nM to 10 mM, 1 nM to 1 mM, or 1 nM to 100 μM.

Exemplary Methods

In some embodiments, a fusion protein is contacted with test compound and a denaturant. In some embodiments, an isolated fusion protein is contacted with a test compound and/or a denaturant. In some embodiments, a fusion protein located within or on a cell, or in a mixture (e.g., a cell lysate) is contacted with a test compound and/or a denaturant. In some embodiments, a fusion protein is contacted with a test compound before or after contacting the fusion protein with a denaturant. In some embodiments, a fusion protein is contacted with a test compound and a denaturant simultaneously, or substantially at the same time. In some embodiments, a fusion protein is expressed in or on a cell (e.g., on a cell surface) and the cell is contacted with a test compound and/or a denaturant.

In certain embodiments, a method comprises contacting a cell with a nucleic acid that encodes or directs the expression of a fusion protein. For example, in some embodiments, a method comprises transfecting or transforming a cell with a nucleic acid (e.g., a vector) that encodes or directs the expression of a fusion protein, followed by contacting the cell, or a lysate of the cell, with a test compound, denaturant, RNase donor and/or a nucleic acid substrate. In certain embodiments, a method comprises introducing a nucleic acid that encodes or directs the expression of a fusion protein into a cell using a suitable method. For example a nucleic acid can be introduced into a eukaryotic cell using a viral vector, or into a bacteria cell using a phage.

In some embodiments, a fusion protein is contacted with a ribonuclease (RNase) donor and/or a nucleic acid substrate. In certain embodiments, a mixture comprising a fusion protein, a test compound and/or a denaturant is contacted with an RNase donor and/or a nucleic acid substrate. In some embodiments, a fusion protein located within or on a cell, or in a mixture (e.g., a cell lysate, e.g., a mixture comprising a fusion protein, a test compound and/or a denaturant) is contacted with an RNase donor and/or a nucleic acid substrate. In some embodiments, a fusion protein is contacted with an RNase donor before or after contacting the fusion protein with a nucleic acid substrate. In some embodiments, a fusion protein is contacted with an RNase donor and a nucleic acid substrate simultaneously, or substantially at the same time.

In certain embodiments, a cell comprising a fusion protein is contacted with a denaturant and a test compound, the denaturant is optionally removed or withdrawn, the cell is exposed to an RNase donor and a nucleic acid substrate, and cleavage of the nucleic acid substrate is detected or quantitated. In some embodiments, a mixture or cell comprising a fusion protein is contacted with a test compound, an RNase acceptor, a nucleic acid substrate and a denaturant substantially at the same time, and cleavage of the substrate is detected and/or quantitated. For example, a cell can be recombinantly produced to express a fusion protein, an RNase donor and/or a nucleic acid substrate, which expression, in some embodiments, is controlled by one or more inducible promoters. The cell, or a lysate thereof, is then contacted with a test compound and a denaturant, e.g., by addition of the test compound and applying heat, while cleavage of a nucleic acid substrate comprising a FRET label is monitored in real time. Variations of such a method are also contemplated herein.

In some embodiments, in a method described herein, the nucleic acid substrate comprises one or more detectable labels. In some embodiments, the nucleic acid substrate comprises one or more FRET labels. In some embodiments, the nucleic acid substrates comprises a pair of FRET labels, wherein the amount of the cleavage product comprises detecting the amount of a fluorescence signal emitted from the cleavage product and obtaining data points, and wherein the fluorescence signal allows for the identification of a target saturation dose, the apparent equilibrium dissociation constant (K_{D}), the half maximal effective concentration (EC50) of target engagement, between the target polypeptide and the test compound.

In some embodiments, the target saturation dose of the test compound is identified by the peak fluorescence value (Emax) after cleavage/depletion of the nucleic acid FRET-labeled substrate in the enzyme reaction. In some embodiments, the apparent equilibrium dissociation constant (K_{D}) between the target polypeptide and the test compound is identified by plotting a saturation binding curve, wherein datapoints beyond the Emax are excluded from the plot. In some embodiments, the EC50 of target engagement is determined from the early datapoints in the reaction where there is excess of the nucleic acid FRET-labeled substrate.

In some embodiments, during or after contacting a fusion protein with heat, an absolute, average or mean temperature of aggregation (T_{agg}) of the fusion protein is determined. In some embodiments, a T_{agg} is determined in the absence of a test compound. In some embodiments, a T_{agg} is determined in the presence of a solvent control, or control compound (e.g., compound known to have no effect on the T_{agg} of the fusion protein, or a compound known to increase a T_{agg} of the fusion protein). Such controls can be used to determine a threshold T_{agg} (e.g., a predetermined threshold), which, in some embodiments, is used to identify compounds that interact with or bind to a target polypeptide. For example, in certain embodiments, a test compound that changes or shifts a T_{agg} of a fusion protein to an amount above a predetermined threshold is often identified as a test compound that interacts with or binds to target polypeptide.

Similar methods can be used to identify a test compound that interacts with or binds to a target polypeptide when electromagnetic radiation, a freeze-thaw or chemical is used as the denaturant. For example, a critical time of exposure, concentration of denaturant, thaw temperature, wavelength, or energy required to denature a fusion protein can be determined in the absence of a test compound and/or in the presence of a control compound to determine a predetermined threshold amount. In some embodiments, any test compound that causes a shift or change in the threshold amount is determined to be a compound that interacts with or binds to the target polypeptide of the fusion protein.

In certain embodiments, a method or assay described herein is conducted as a multiplex assay and/or a high-throughput assay comprising conducting the method in at least 96, at least 100, at least 384, at least 500, at least 1000, at least 1536, at least 5000, or at least 10,000 separate vessels. In certain embodiments, a method or assay described herein is conducted as a multiplex assay and/or a high-throughput assay wherein some or all of the steps of the method are conducted substantially simultaneously, or at the same time in a plurality of vessels. In some embodiments, some or all of a plurality of separate vessels used in a multiplex assay and/or a high-throughput assay is contacted with, or comprises, a different fusion protein, a different denaturant, a different test compound, a different RNase acceptor, and/or a different nucleic acid substrate. In some embodiments, some or all of a plurality of separate vessels used in a multiplex assay and/or a high-throughput assay is contacted with, or comprises, the same fusion protein, the same denaturant, the same test compound, the same RNase acceptor, and/or the same nucleic acid substrate. A “vessel” or “container” as used herein, refers to any suitable container, vessel, tube or a well. A vessel can be a well, for example a well in a microtiter plate.

In certain embodiments, a method or assay described herein is conducted as a multiplex assay and/or a high-throughput assay using an array of surface-bound fusion proteins often located at addressable location on a suitable substrate (e.g., a chip). In some embodiments, an array comprises at least 20, at least 96, at least 100, at least 384, at least 500, at least 1000, at least 1536, at least 5000, or at least 10,000 different fusion proteins bound to the surface of a suitable substrate.

Methods and assays described herein provide for increased responsiveness and sensitivity over other types of complementation assays. The methods provided herein can detect the presence of an active test compound at ng levels.

### EXAMPLES

### Example 1

Below is an example of a method for real-time cellular drug-target engagement. This example may be performed at least in part according to method 100 shown in FIG. 1, at least in part by one or more components of system 10 shown in FIG. 2, and/or in other ways.

### Materials: Reagents, Cell Lines, and Constructs

The following antibodies were obtained from Cell Signaling Technology: Rabbit mAb S-Tag (D2K2V) XP (Cat #12774), anti-MTH1 (D6V40) Rabbit mAb (Cat #43918). HEK-293 cells were from ATCC (Cat #CRL-1573) and were cultured in DMEM (Millipore SIGMA Cat #D5796) supplemented with 10% Fetal Bovine Serum (FBS) (Millipore SIGMA Cat #F2442), and 1× Penicillin/Streptomycin (Millipore SIGMA Cat #P4333). Trypsin-EDTA solution 1× (0.05% trypsin, 0.02% EDTA) was from Millipore SIGMA (Cat #59417C) and 20×TBS solution was from Teknova (Cat #T1680). Triton-X-100 was from Millipore SIGMA (Cat #X100). The MTH1 inhibitor (S) Crizotinib was from Millipore SIGMA (Cat #PZ0240). DMSO was from Millipore SIGMA (Cat #673439). The 96 well plates were from CELLTREAT Scientific (Cat #229196); white PCR plates were from BIORAD (cat #MLL9651) and the Microseal ‘B’ film for sealing the PCR plates was from BIORAD (Cat #MSB1001); black 96 well plates from COSTAR (Cat #3915). Transfection of the cells was using Lipofectamine 2000 transfection reagent (Thermo Fisher Scientific Cat #11668027) according to manufacturer recommended protocol. The optimized fluorogenic substrate (5′-6FAM/ArUAA/3′ TAMRA_(NHS ester) (v3)) was purchased from IDT. The S-Protein was cloned with 6×His tag at the carboxy terminus into vector pET-30a(+) for bacterial expression using KPN1 GGTACC and BamH1 GGATCC cloning sites by Synbio Technologies Inc. The S-protein His tag fusion protein was expressed and purified from bacteria by Synbio Technologies Inc.

The sequence of the cloned construct encoding the RNase donor was as follows:

GGTACCATGAGCAGCTCCAACTACTGTAACCAGATGATGAAGAGCCGGAAC CTGACCAAAGATCGATGCAAGCCAGTGAACACCTTTGTGCACGAGTCCCTGGCTGAT GTCCAGGCCGTGTGCTCCCAGAAAAATGTTGCCTGCAAGAATGGGCAGACCAATTG CTACCAGAGCTACTCCACCATGAGCATCACCGACTGCCGTGAGACCGGCAGCTCCA AGTACCCCAACTGTGCCTACAAGACCACCCAGGCGAATAAACACATCATTGTGGCTT GTGAGGGAAACCCGTACGTGCCAGTCCACTTTGATGCTTCAGTGCATCACCATCAC CATCACTAGGGATCC (SEQ ID NO:22). The sequence encoding the 6×His tag is bold and the TAG stop codon is underlined.

The corresponding translated amino acid sequence of the His tagged RNase donor protein (S protein) was:

(SEQ ID NO: 23)
MSSSNYCNQMMKSRNLTKDRCKPVNTFVHESLADVQAVCSQKNVACKN
GQTNCYQSYSTMSITDCRETGSSKYPNCAYKTTQANKHIIVACEGNPY
VPVHFDASVHHHHHH.

Cloning of the S-tag acceptor peptide with the mutT homologue (MTH1) protein (i.e., the target polypeptide) was performed by Synbio Technologies Inc. Cloning of the S-tag acceptor peptide to the carboxy terminal of MTH1 was as follows in vector pcDNA3.1(+): cloning site KPN1 GGTACC and BamH1 GGATCC (bold).

We generated the encoding construct with no linker between MTH1 and the S-tag peptide (underlined) as shown below:

(SEQ ID NO: 24)
GGTACCATGGGCGCCTCCAGGCTCTATACCCTGGTGCTGGTCCTGCAG
CCTCAGCGAGTTCTCCTGGGCATGAAAAAGCGAGGCTTCGGGGCCGGC
CGGTGGAATGGCTTTGGGGGCAAAGTGCAAGAAGGAGAGACCATCGAG
GATGGGGCTAGGAGGGAGCTGCAGGAGGAGAGCGGTCTGACAGTGGAC
GCCCTGCACAAGGTGGGCCAGATCGTGTTTGAGTTCGTGGGCGAGCCT
GAGCTCATGGACGTGCATGTCTTCTGCACAGACAGCATCCAGGGGACC
CCCGTGGAGAGCGACGAAATGCGCCCATGCTGGTTCCAGCTGGATCAG
ATCCCCTTCAAGGACATGTGGCCCGACGACAGCTACTGGTTTCCACTC
CTGCTTCAGAAGAAGAAATTCCACGGGTACTTCAAGTTCCAGGGTCAG
GACACCATCCTGGACTACACACTCCGCGAGGTGGACACGGTCAAGGAA
ACTGCAGCAGCCAAGTTTGAGCGGCAGCACATGGACTCCAGCACTTCC
GCTGCCTAGGCTGCCTAGGGATCC.

The translated amino acid sequence for resulting fusion protein comprising the MTH1 and carboxy terminal S-tag acceptor peptide is shown below. The 20 amino acid sequence of the S-tag acceptor peptide is underlined. It was determined that the S-tag can be shortened to the first 15 amino acids and still act as an S-tag acceptor peptide.

(SEQ ID NO: 25)
MGASRLYTLVLVLQPQRVLLGMKKRGFGAGRWNGFGGKVQEGETIEDG
ARRELQEESGLTVDALHKVGQIVFEFVGEPELMDVHVFCTDSIQGTPV
ESDEMRPCWFQLDQIPFKDMWPDDSYWFPLLLQKKKFHGYFKFQGQDT
ILDYTLREVDTVKETAAAKFERQHMDSSTSAA.

We also generated an encoding construct for a fusion protein comprising a 3 amino acid linker (boxed) between the MTH1 target polypeptide and the S-tag acceptor peptide (underlined) as shown below:

(SEQ ID NO: 26)
GGTACCATGGGCGCCTCCAGGCTCTATACCCTGGTGCTGGTCCTGCAGCC
TCAGCGAGTTCTCCTGGGCATGAAAAAGCGAGGCTTCGGGGCCGGCCGGT
GGAATGGCTTTGGGGGCAAAGTGCAAGAAGGAGAGACCATCGAGGATGGG
GCTAGGAGGGAGCTGCAGGAGGAGAGCGGTCTGACAGTGGACGCCCTGCA
CAAGGTGGGCCAGATCGTGTTTGAGTTCGTGGGCGAGCCTGAGCTCATGG
ACGTGCATGTCTTCTGCACAGACAGCATCCAGGGGACCCCCGTGGAGAGC
GACGAAATGCGCCCATGCTGGTTCCAGCTGGATCAGATCCCCTTCAAGGA
CATGTGGCCCGACGACAGCTACTGGTTTCCACTCCTGCTTCAGAAGAAGA
AATTCCACGGGTACTTCAAGTTCCAGGGTCAGGACACCATCCTGGACTAC

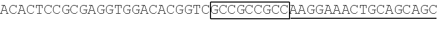

CAAGTTTGAGCGGCAGCACATGGACTCCAGCACTTCCGCTGCCTAGGCTG
CCTAGGGATCC

The translated amino acid sequence for the resulting fusion protein comprising the MTH1 target polypeptide with a 3 amino acid spacer and a carboxy terminal S-tag acceptor peptide is shown below. The S-tag is underlined and the 3 amino acid spacer is boxed.

(SEQ ID NO: 27)
MGASRLYTLVLVLQPQRVLLGMKKRGFGAGRWNGFGGKVQEGETIEDGAR
RELQEESGLTVDALHKVGQIVFEFVGEPELMDVHVFCTDSIQGTPVESDE
MRPCWFQLDQIPFKDMWPDDSYWFPLLLQKKKFHGYFKFQGQDTILDYTL

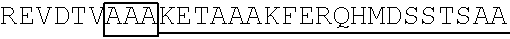

An encoding construct comprising a 10 amino acid linker (boxed) between the MTH1 target polypeptide and S-tag acceptor peptide (underlined) was also constructed as shown below:

(SEQ ID NO: 28)
GGTACCATGGGCGCCTCCAGGCTCTATACCCTGGTGCTGGTCCTGCAGCC
TCAGCGAGTTCTCCTGGGCATGAAAAAGCGAGGCTTCGGGGCCGGCCGGT
GGAATGGCTTTGGGGGCAAAGTGCAAGAAGGAGAGACCATCGAGGATGGG
GCTAGGAGGGAGCTGCAGGAGGAGAGCGGTCTGACAGTGGACGCCCTGCA
CAAGGTGGGCCAGATCGTGTTTGAGTTCGTGGGCGAGCCTGAGCTCATGG
ACGTGCATGTCTTCTGCACAGACAGCATCCAGGGGACCCCCGTGGAGAGC
GACGAAATGCGCCCATGCTGGTTCCAGCTGGATCAGATCCCCTTCAAGGA
CATGTGGCCCGACGACAGCTACTGGTTTCCACTCCTGCTTCAGAAGAAGA
AATTCCACGGGTACTTCAAGTTCCAGGGTCAGGACACCATCCTGGACTAC

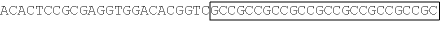

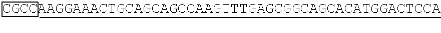

GCACTTCCGCTGCCTAGGCTGCCTAGGGATCC.

The translated amino acid sequence of the MTH1 target polypeptide with a 10 amino acid spacer and a carboxy terminal S-tag acceptor peptide is shown below. The S-tag is underlined and the 10 amino acid spacer is boxed.

(SEQ ID NO: 29)
MGASRLYTLVLVLQPQRVLLGMKKRGFGAGRWNGFGGKVQEGETIEDGAR
RELQEESGLTVDALHKVGQIVFEFVGEPELMDVHVFCTDSIQGTPVESDE
MRPCWFQLDQIPFKDMWPDDSYWFPLLLQKKKFHGYFKFQGQDTILDYTL

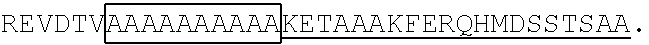

### Methods: Protocol for Assessing Drug Target Engagement Using the S-Tag System

A. Test Cell Numbers in Kinetic Read

Transfect HEK-293 cells were transfected with an MTH1-S-tag fusion construct using Lipofectamine 2000. This protocol was improved using reverse transfection whereby the cells were lifted and re-plated with the transfection reagent along with DNA. Twenty-four hours after transfection, the cells were lifted with trypsin, washed with 1×TBS and cells were counted. A dilution series of cell number was tested.

Cells were diluted to 1×10⁶ cells/ml (i.e. 1000 cells/μl) in 1×TBS (cold TBS). Cell counts in a range of 50,000 cells down to 1000 cells were tested in a final volume of 50 μl with cold TBS in 96 well plates. To each well containing 50 μl cells, 50 μl of room temp 1%-Triton-X 100 in TBS was added to get a final of concentration of 0.5% Triton-X-100. Wells were mixed by pipetting up down and allowed to incubate for 10 min. at room temp to gently lyse the cell membranes. 80 μl from each well was transferred to a black costar plate while avoiding air bubbles, and 10 μl of a 10×S-protein (RNase acceptor) was added to a final concentration of 1 ng/μl, followed by addition of 10 μl of 10× Substrate (nucleic acid substrate) to get a final of substrate concentration of 50 nM. 10×S Protein (10 ng/μl) was prepared at room temp in 0.5% triton-X in TBS just prior to use. 10× Substrate (500 nM) is prepared at room temp with dH₂0 just prior to use.

The plate was read immediately in kinetic mode (excitation=430 nm, emission=520 nm) with the PolarStar Omega 96-well plate reader. Every plate was read every min for 10 min (11 time points were generated). The appropriate dilution of cells was selected that gave a highest signal without saturating the signal within 6 min.

B. Identify T_{agg} for the Experiment by Running Thermal Gradient

Cells were diluted to 1×10⁶ cells/ml (1000 cells/ul) in 1×TBS (cold TBS).

The correct dilution of cells was prepared in a final volume of 50 μl with cold TBS in a white PCR plate. The plates were exposed to a thermal gradient from 40° C. to 64° C. for 3 min. using a thermal cycler (MJ Research PTC-200 Peltier Thermal Cycler) and allowed to sit at room temp for 3 min, followed by addition of 50 μl of room temp 1% Triton-X-100 in TBS to get final of 0.5% Triton-X-100. The wells were mixed by pipetting up down followed by incubation for 10 min at room temp.

80 ul of lysate from each well was transferred to a black costar plate while avoiding air bubbles, followed by addition of 10 μl of 10×S protein (RNase acceptor) to get a final concentration of 1 ng/μl, followed by 10 μl of 10× Substrate (nucleic acid substrate) to get a final concentration of 50 nM. The plate was read immediately in kinetic mode (ex=430 nm, em=520 nm) with the PolarStar Omega 96-well plate reader. The plate was read every min for 10 min. The T_{agg} for the MTH1-S-tag fusion protein was determined to be about 50° C.

C. Test Inhibitor Doses for the Given Cell Number and T_{agg}

The inhibitor (i.e., test compound) doses were tested using the optimal cell number and T_{agg} determined from the above experiment. Cells were diluted to 1×10⁶ cells/ml (1000 cells/ul) in 1×TBS (cold TBS). The correct dilution of cells was prepared in a final volume of 50 ul with cold TBS in white PCR plates. 0.25 ul of 200× Crizotinib (a multitargeted small molecule tyrosine kinase inhibitor) in DMSO was added (or other inhibitor to be tested). For Crizotinib the EC50 was tested in a range of approximately 50 nM. Control wells included DMSO tested at the T_{agg} (50° C.) (the 0% stability control) and DMSO tested at 40° C. (the 100% stability control).

Cells were incubated with inhibitor 40 min. on ice, then placed in a thermal cycler at T_{agg} for 3 min. (Control wells were heated separately at 40° C. for 3 min), followed by room temp for 3 min, followed by addition of 50 ul of room temp 1% Triton-X-100 in TBS to get final of 0.5% Triton-X-100. Cells were mixed by pipetting up down followed by incubation for 10 min at room temp. 80 μl was transferred to black costar plate while avoiding air bubbles, followed by addition of 10 μl of 10×S protein to get a final of 1 ng/μl, and 10 μl of 10× Substrate to get a final of 50 nM. Plates were read immediately in kinetic mode (ex=430 nm, em=520 nm) with the PolarStar Omega 96-well plate reader, every min for 10 min. Fluorescence units were plotted with corresponding inhibitor (test compound) dose to determine the EC50 for protein stabilization.

### Results

HEK293 mammalian cells were transfected with the three MTH1-S-tag peptide fusion protein constructs to determine if spacer length between target polypeptide and the S-tag acceptor peptide impacts the detection of functional RNase S complementation. FIG. 22 shows equal expression of the constructs in the HEK293 cells as determined by Western blot.

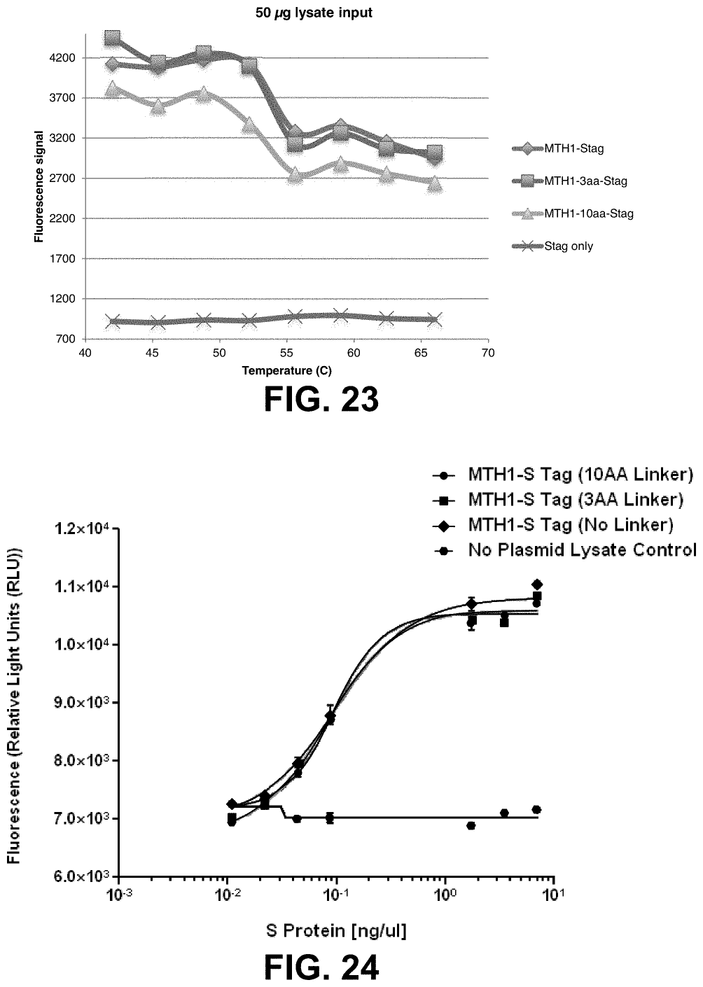

Testing 50 μg of transfected cells lysate input and comparing the different lengths of the spacer showed that there was no significant effect of spacer length on fluorescent signal (FIG. 23). Spacer length did not appear to play a significant role in signal levels or temperature of aggregation (T_{agg}) of the target protein. Structural and inertness and thermal insensitivity of the S-tag did not contribute to the biophysical stability of the target protein. This greatly improved downstream applications of this S-tag technology, and its use with a wide range of target proteins. Also, the data showed that in the absence of expression of S-tag fusion there was no detectable signal demonstrating specificity of the S-tag S protein complementation.

To determine if S protein concentration impacts fluorescent signal, increasing concentration of S protein with the different S-tag fusions was tested. FIG. 24 shows that S protein concentration started to saturate signal at about 1-2 ng/ul S protein. The data demonstrated the sensitivity of the system, as it requires only minimal levels of S protein to drive enzyme complementation.

Similarly increasing concentration of the FRET nucleic acid substrate was tested and it was observed that the ideal concentration was in a range of 50 nM or more (Table 1).

| | | FRET Probe (nM) | | | | | | | | | | | |
|---|---|---|---|---|---|---|---|---|---|---|---|---|---|
| | | 600 | 200.00 | 66.67 | 22.22 | 7.41 | 2.47 | 0.82 | 0.27 | 0.09 | 0.03 | 0.01 | 0 |
| S protein | 6 | 14525 | 4220 | 580 | 78 | 76 | 74 | 74 | 75 | 70 | 68 | 68 | 70 |
| (ng/μl) | 2.00 | 13242 | 3609 | 639 | 77 | 76 | 73 | 73 | 73 | 72 | 70 | 71 | 70 |
| | 0.67 | 1147 | 430 | 122 | 70 | 73 | 72 | 71 | 70 | 71 | 71 | 70 | 69 |
| | 0.22 | 549 | 225 | 98 | 73 | 69 | 70 | 71 | 71 | 70 | 71 | 70 | 69 |
| | 0.07 | 397 | 171 | 85 | 74 | 72 | 73 | 72 | 72 | 71 | 74 | 70 | 71 |
| | 0.02 | 365 | 159 | 89 | 71 | 73 | 73 | 74 | 72 | 72 | 71 | 71 | 70 |
| | 0.01 | 367 | 154 | 88 | 72 | 72 | 72 | 73 | 74 | 72 | 71 | 72 | 68 |
| | 0 | 349 | 144 | 85 | 69 | 70 | 71 | 70 | 71 | 69 | 71 | 70 | 68 |

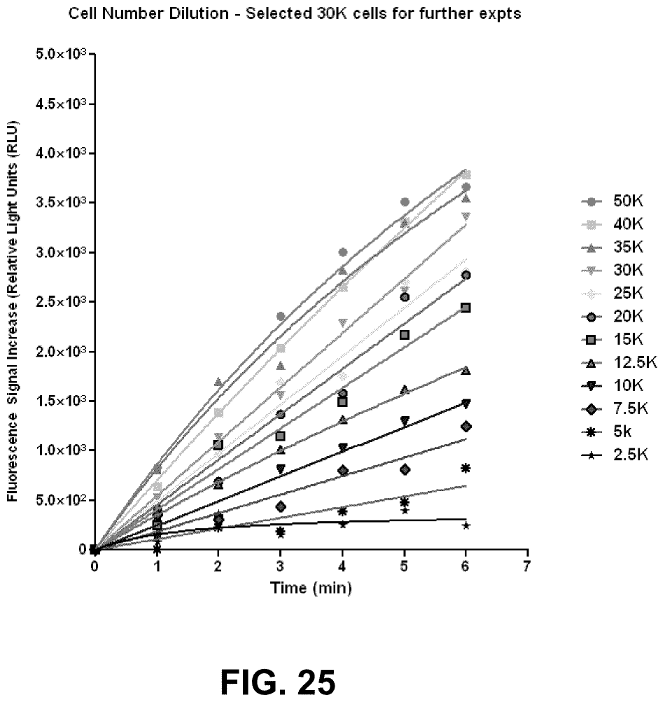

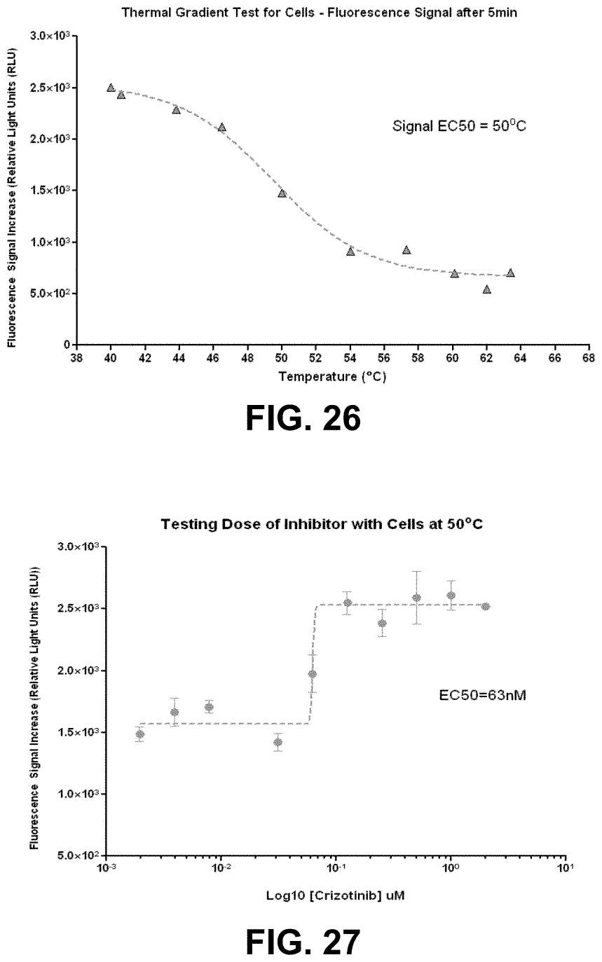

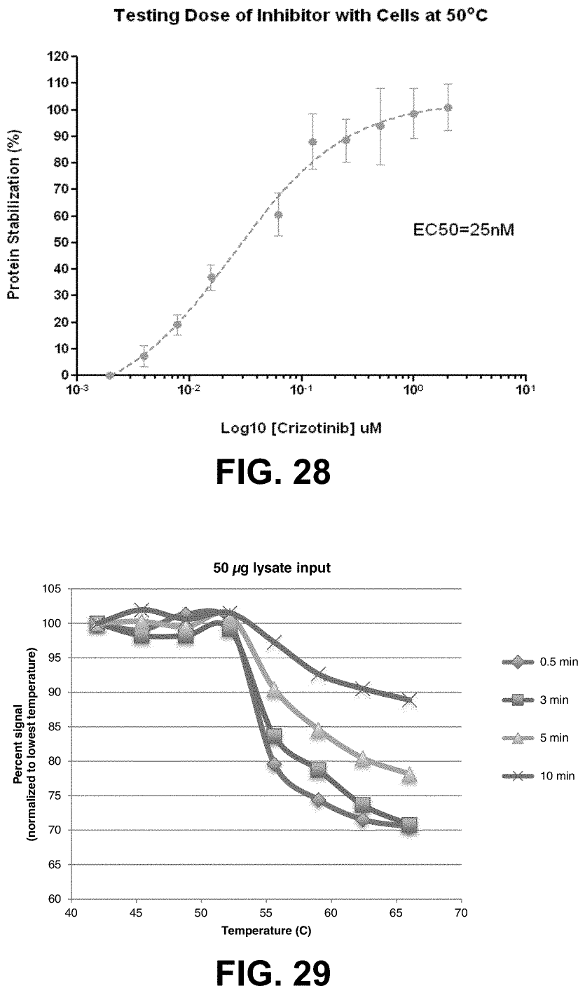

The kinetics of the cleavage of the fluorogenic substrate was rapid and detection was sensitive. FIG. 25 showed a variation of fluorescent signal with respect to cell number. Once optimal cell numbers were determined then the T_{agg} for the fusion protein was identified by performing a modified cellular-thermal shift assay (CTSA). FIG. 26 demonstrated that for the MTH1-S-tag fusion, the T_{agg} was approximately 50° C. Using the T_{agg}, a dose response curve was performed to identify binding of the small molecule Crizotinib to the fusion protein. Cells transfected with the MTH1-S-tag fusion protein were incubated with the MTH1 inhibitor (S) Crizotinib at increasing concentrations (FIG. 27). Crizotinib stabilized the MTH1 fusion protein as indicated by an increase in fluorescence signal (FIG. 27). The EC50 was determined using the formula for protein stabilization shown below and indicated an EC50 for protein stabilization of approximately 25 nM (FIG. 28).

Formula for protein stabilization:

(1−((RLU_{40° C.}−RLU_{50° C.})−(RLUₓ−RLU_{50° C.}))/(RLU_{40° C.}−RLU_{50° C.})))*100

where RLU is relative light units.

TABLE 2
-Signal detected without centrifugation after heat challenge. Fluorescence
was detected at wavelength ex = 475 nm/em = 500-550 nm.

| | | Fluorescent reagent (nM) | | | | | | | | | | | |
|---|---|---|---|---|---|---|---|---|---|---|---|---|---|
| | | 120.000 | 40.000 | 13.333 | 4.444 | 1.481 | 0.494 | 0.165 | 0.055 | 0.018 | 0.006 | 0.002 | 0 |
| S protein | 1.2 | 14525 | 4220 | 580 | 78 | 76 | 74 | 74 | 75 | 70 | 68 | 68 | 70 |
| (ng/μl) | 0.4 | 13242 | 3609 | 639 | 77 | 76 | 73 | 73 | 73 | 72 | 70 | 71 | 70 |
| | 0.134 | 1147 | 430 | 122 | 70 | 73 | 72 | 71 | 70 | 71 | 71 | 70 | 69 |
| | 0.044 | 549 | 225 | 98 | 73 | 69 | 70 | 71 | 71 | 70 | 71 | 70 | 69 |
| | 0.014 | 397 | 171 | 85 | 74 | 72 | 73 | 72 | 72 | 71 | 74 | 70 | 71 |
| | 0.004 | 365 | 159 | 89 | 71 | 73 | 73 | 74 | 72 | 72 | 71 | 71 | 70 |
| | 0.002 | 367 | 154 | 88 | 72 | 72 | 72 | 73 | 74 | 72 | 71 | 72 | 68 |
| | 0 | 349 | 144 | 85 | 69 | 70 | 71 | 70 | 71 | 69 | 71 | 70 | 68 |

TABLE 3
-Signal detected with centrifugation after heat challenge. Fluorescence was detected at wavelength
ex = 475 nm/em = 500-550 nm. Plate was centrifuged at 12 K rpm after heat challenge.

| | | Fluorescent reagent (nM) | | | | | | | | | | | |
|---|---|---|---|---|---|---|---|---|---|---|---|---|---|
| | | 120.000 | 40.000 | 13.333 | 4.444 | 1.481 | 0.494 | 0.165 | 0.055 | 0.018 | 0.006 | 0.002 | 0 |
| S protein | 1.2 | 15334 | 4592 | 780 | 290 | 201 | 144 | 129 | 92 | 75 | 70 | 67 | 65 |
| (ng/μl) | 0.4 | 14417 | 3959 | 809 | 242 | 170 | 145 | 118 | 89 | 75 | 71 | 70 | 66 |
| | 0.134 | 1254 | 397 | 126 | 97 | 83 | 77 | 75 | 73 | 69 | 71 | 70 | 68 |
| | 0.044 | 618 | 248 | 99 | 76 | 75 | 77 | 72 | 71 | 70 | 71 | 71 | 68 |
| | 0.014 | 437 | 184 | 89 | 72 | 72 | 71 | 72 | 72 | 71 | 71 | 70 | 66 |
| | 0.004 | 393 | 179 | 92 | 74 | 74 | 73 | 72 | 71 | 70 | 69 | 69 | 68 |
| | 0.002 | 392 | 164 | 89 | 73 | 72 | 71 | 69 | 71 | 71 | 69 | 66 | 67 |
| | 0 | 374 | 145 | 86 | 73 | 72 | 72 | 70 | 69 | 68 | 69 | 67 | 65 |

*Note that there is no difference is signal levels in Tables 2 and 3. Removal of the centrifugation step speeds up signal detection, reduces time spent between sample dispensing and signal reading, and importantly greatly improves downstream applications of this S-tag technology.

Short incubation time of lysate with S-protein and substrate was sufficient for complete signal development (FIG. 29). The shortest incubation time (0.5 min) is sufficient for complete signal development. At longer incubation times signal quality is not compromised. Such fast signal development is important for downstream application of this technology, particularly for array and chip applications.

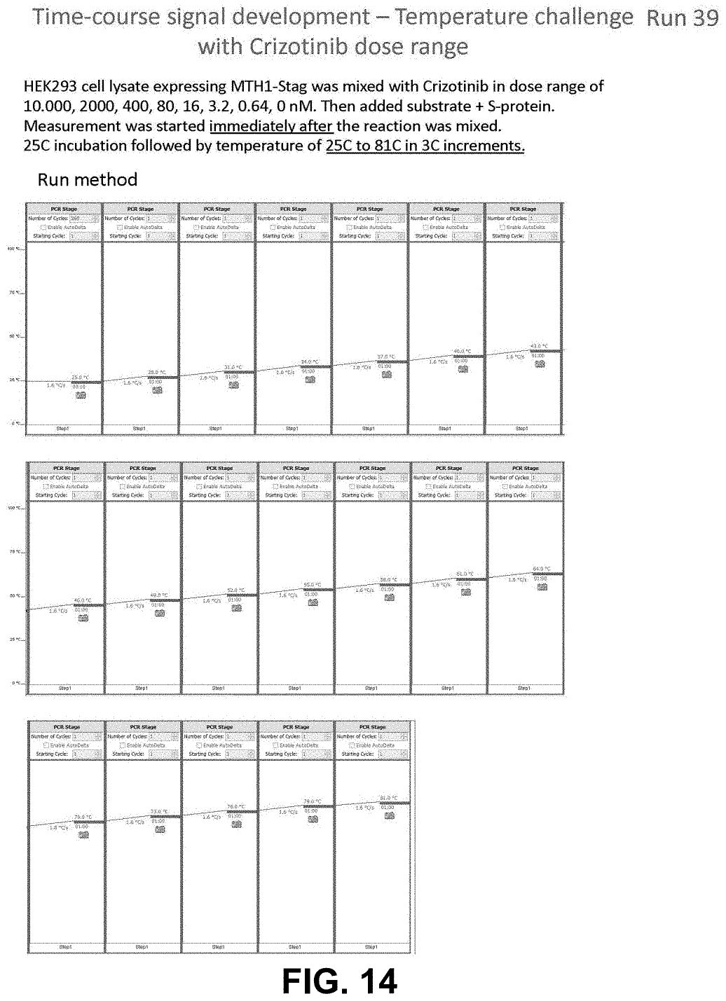

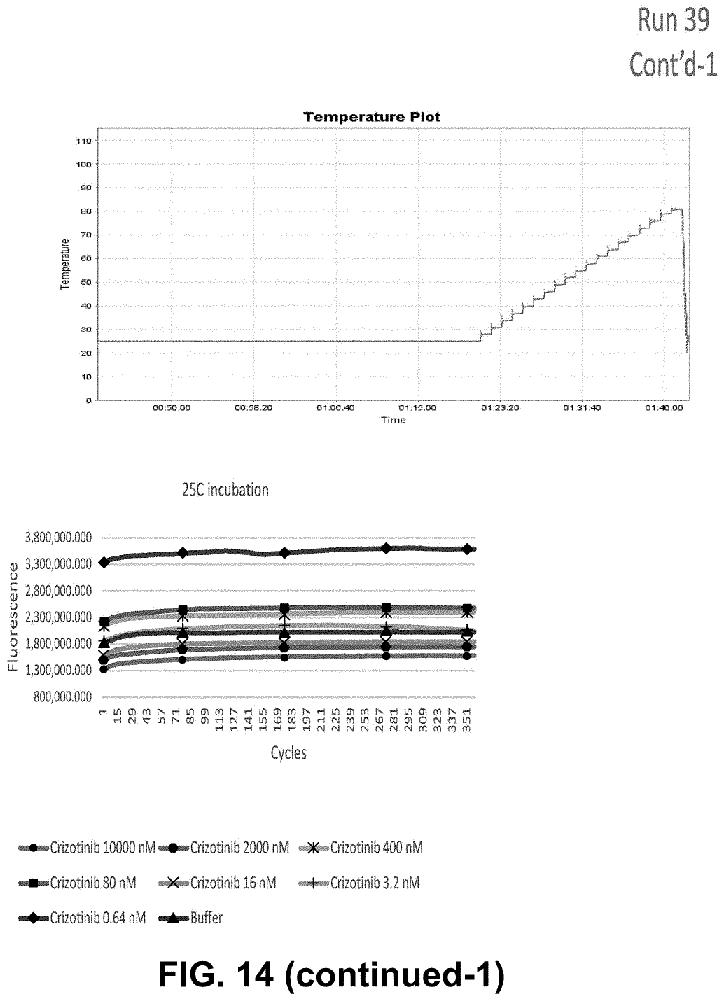

Low lysate/cell input in combination with short incubation times yielded sensitive signal demonstrating a melting profile of the target protein when engaged with its ligand (FIGS. 14 & 15). Signal pattern between high and low lysate inputs was identical for Crizotinib-MTH1 engagement. These figures demonstrate persistence of a signal pattern at longer incubation times, despite signal saturation. Such flexibility of lysate/cell input in combination with incubation time can be used to detect engagement EC50 of ligands of various target engagement potencies.

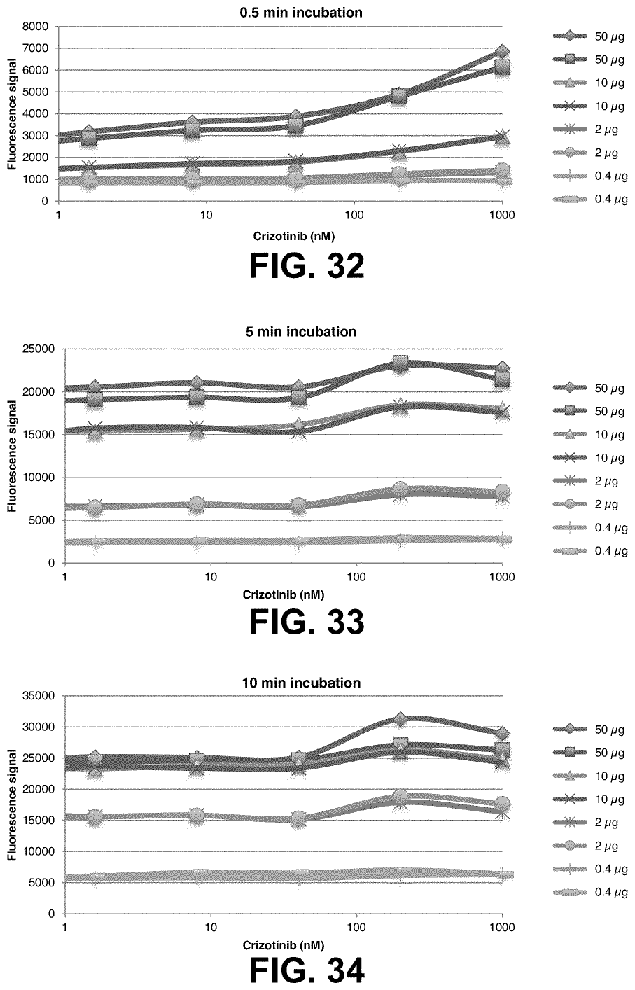

FIGS. 32-34 demonstrate the effect of lysate/cell input in combination with incubation times on signal pattern and persistence, demonstrating melting profiles of the target protein when engaged with its ligand. Note that signal pattern between short and long incubation times was identical for Crizotinib-MTH1 engagement (FIGS. 32-34). Note persistence of signal pattern at longer incubation times, despite signal saturation. Also note wide signal separation at short incubation time of 0.5 min, and narrower signal separation at longer incubations. Also note reciprocity between lysate input and incubation time. Such flexibility of lysate/cell input in combination with incubation time can be used to detect engagement EC50 of ligands of various target engagement potencies, as well as various applications where various amounts of lysate input or incubation times may be preferred.

### Example 2

In one embodiment of a method or assay described herein, a target polypeptide is a protein, or modified protein thereof, encoded by a pathogen (non-limiting examples include coronaviruses, influenza virus, hepatitis virus, and even bacteriophages). The fusion protein comprises the target pathogen protein expressed with an N-terminal and/or C-terminal S-tag (e.g., a S-tag acceptor peptide). An expression construct for the pathogen protein would include a codon optimized cDNA to allow for maximum expression of the fusion protein. In this example, an array is used such that DNA encoding the fusion protein is provided to cells in a multi-well format (96 or 384 or 1536 well format) for expression in a cell line of interest. The DNA can be transfected into cells located in each of the wells of a multi-well plate to allow for protein expression. The plate array can be used to test single or multiple agents (drugs, i.e., test compounds) on all of the fusion proteins in a single run. In addition, the array can be tested with multiple doses of a single or multiple test compounds. The array can be used in a high-throughput method for testing compound libraries on the entire proteome of a pathogen. The array can be used to screen test compound binding under various physiological conditions (e.g., different buffers, presence of serum components, growth factors, or other stimulants). The plate can then be put through a gradient heating denaturation step followed by addition of the RNase donor for enzyme complementation and FRET substrate cleavage detection. Fluorescence detection using a plate reader would be performed in kinetic mode to monitor any potential increase in fluorescence over time.

### Example 3

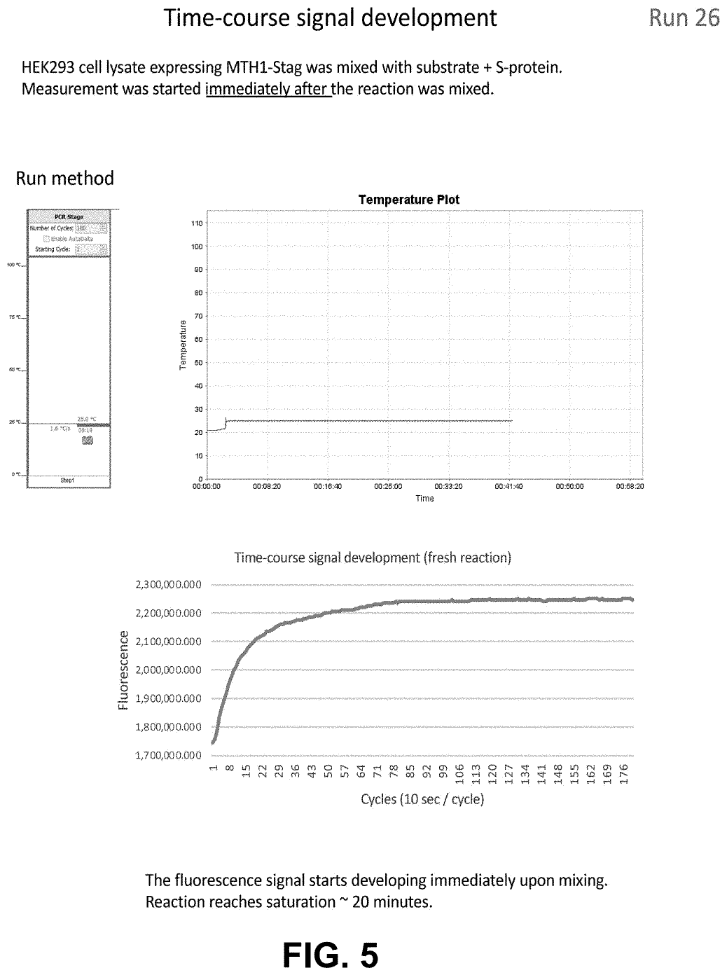

### FIG. 5: Time Course Signal Development.

HEK293 cell were transfected with DNA encoding for MTH1-Stag fusion. 96-well RT-QPCR reaction plate was used to set up the target engagement reactions. HEK293 cells expressing MTH1-Stag fusion protein were lysed and complemented with nuclease substrate+S-protein. Measurement of fluorescence signal was started immediately in RT-QPCR machine after the reaction was mixed.

Run in RT-QPCR machine was programmed as a 1-step reaction composed of multiple 10 sec cycles, each at 25° C. (top left figure). Temperature plot (top right) and fluorescence signal development in real time (shown as cycles, bottom right) are shown. Real-time signal development was measured over the course of 176 cycles (each 10 sec). Liner phase and plateau phase of signal development were observed.

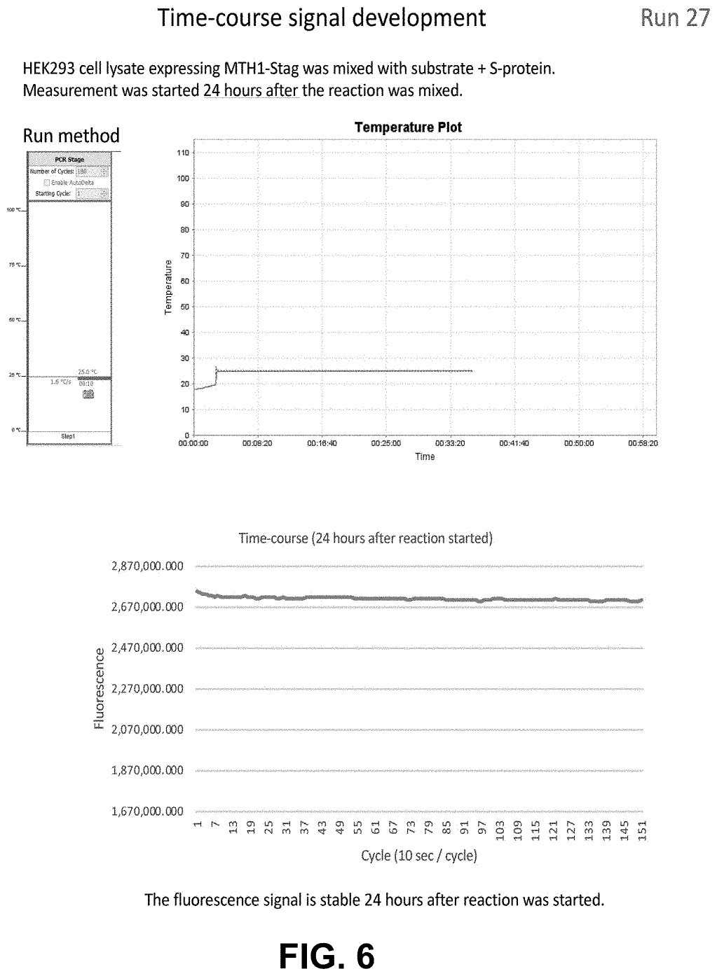

FIG. 6: Time Course Signal Development (Reaction after 24 Hours).

Continuation of the reaction started in FIG. 5, 24 hours after the start. Same run method was used as described in FIG. 5.

Run in RT-QPCR machine was programmed as a 1-step reaction composed of multiple 10 sec cycles, each at 25° C. (top left figure). Temperature plot (top right) and fluorescence signal development in real time (shown as cycles, bottom right) are shown. Real-time signal development was measured over the course of 151 cycles (each 10 sec). The fluorescence signal is stable after 24 hours after the reaction was started.

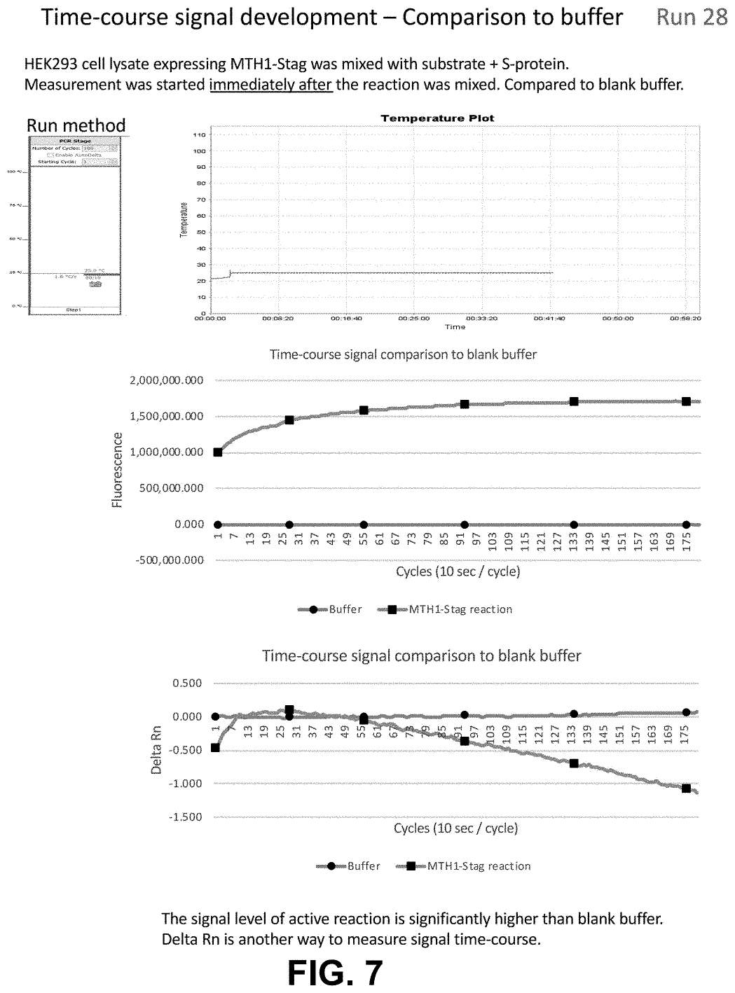

### FIG. 7: Time-Course Signal Development—Comparison to Buffer.

HEK293 cell were transfected with DNA encoding for MTH1-Stag fusion. 96-well RT-QPCR reaction plate was used to set up the target engagement reactions. HEK293 cells expressing MTH1-Stag fusion protein were lysed and complemented with nuclease substrate+S-protein. Measurement of fluorescence signal was started immediately in RT-QPCR machine after the reaction was mixed. Signal was compared to blank buffer.

Run in RT-QPCR machine was programmed as a 1-step reaction composed of multiple 10 sec cycles, each at 25° C. (top left figure). Temperature plot (top right) and fluorescence signal development in real time (shown as cycles, middle right) and Delta Rn of fluorescence signal (bottom right) are shown. Real-time signal development was measured over the course of 175 cycles (each 10 sec). Liner phase and plateau phase of signal development are observed for the MTH1-Stag complemented reaction, while no signal is detected for the buffer.

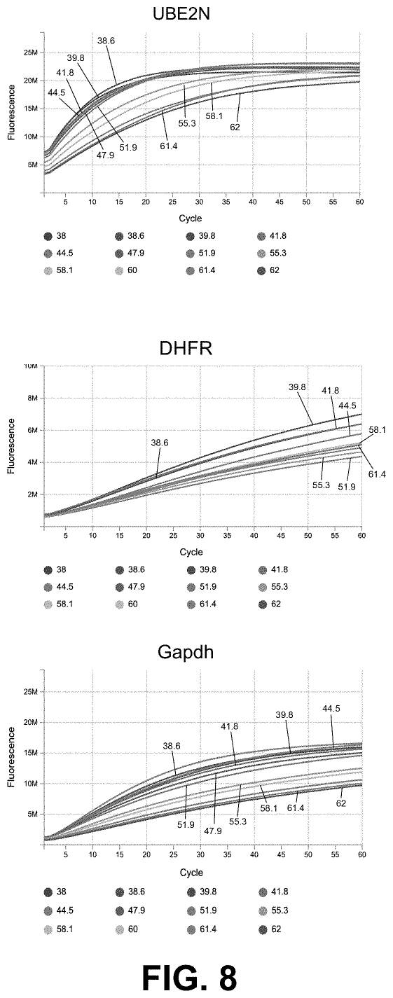

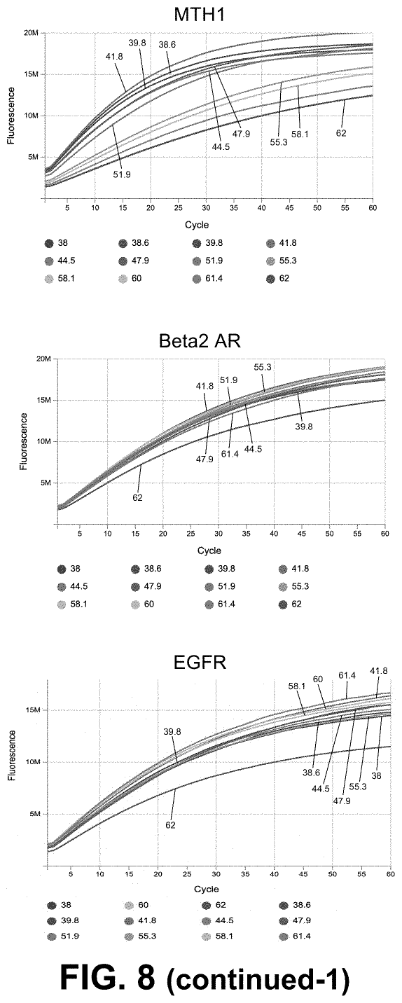

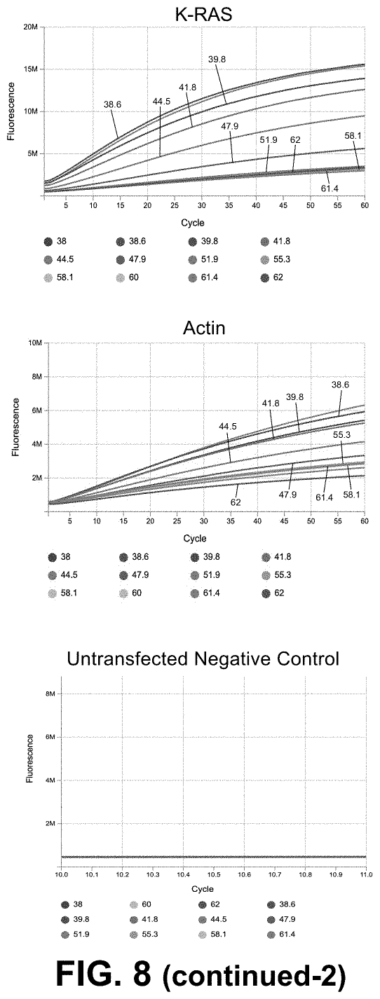

FIG. 8. Real-Time Detection of Fluorescence from the Enzyme Complementation Assay with QuantStudio 3 qPCR Real-Time System.

Constructs with Stag fusions were expressed in HEK293 cells, non-denaturing lysates isolated and heated at the indicated temperatures ranging from 38° C. to 62° C., then assayed for fluorescence to quantitate protein level.

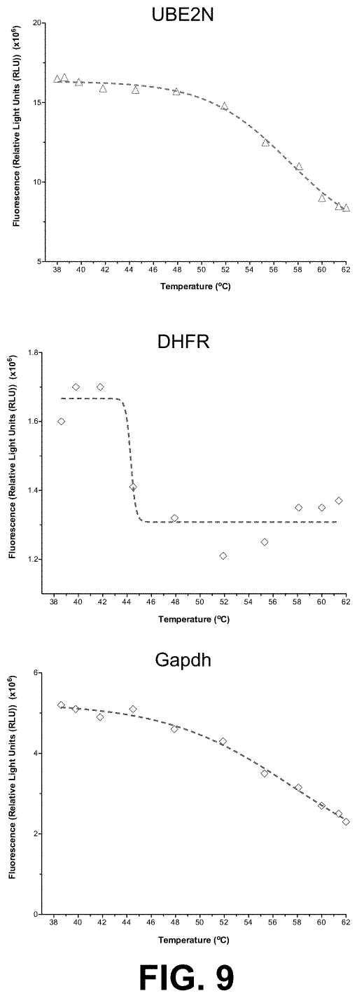

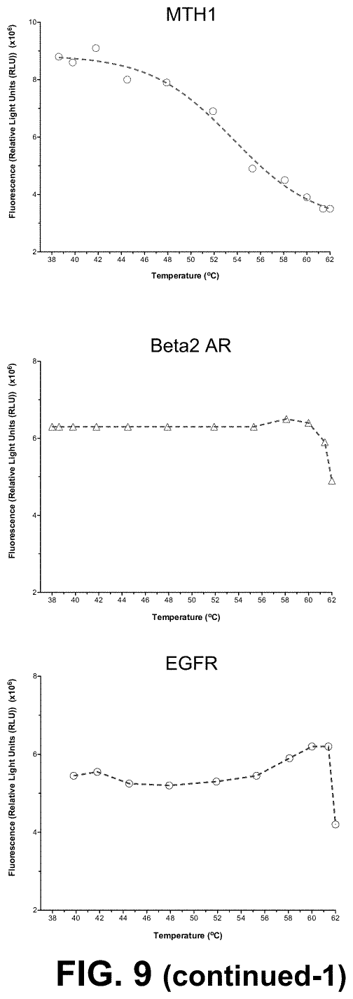

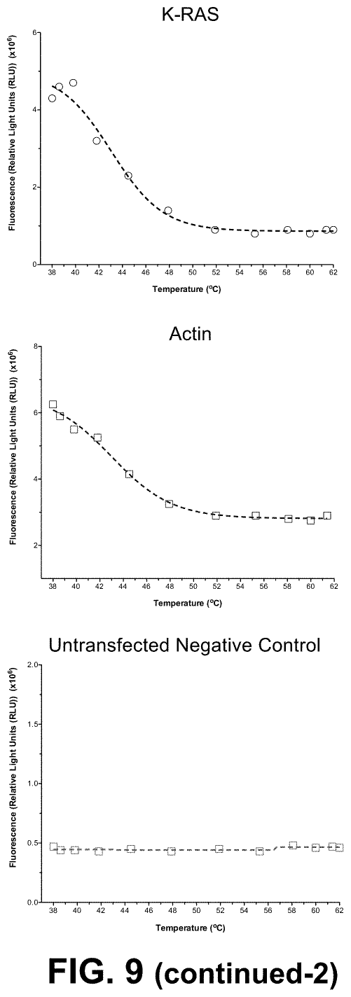

### FIG. 9. Thermal Profiles for the Fusion Constructs Examined in FIG. 1.

Fluorescence (relative light units) (×10⁶) from the linear part of the curves shown in FIG. 1 were plotted with temperature tested. Non-linear regression analysis was used to plot a dose-response curve. For both Beta2 AR and EGFR the temperature of aggregation is superior to 62° C.

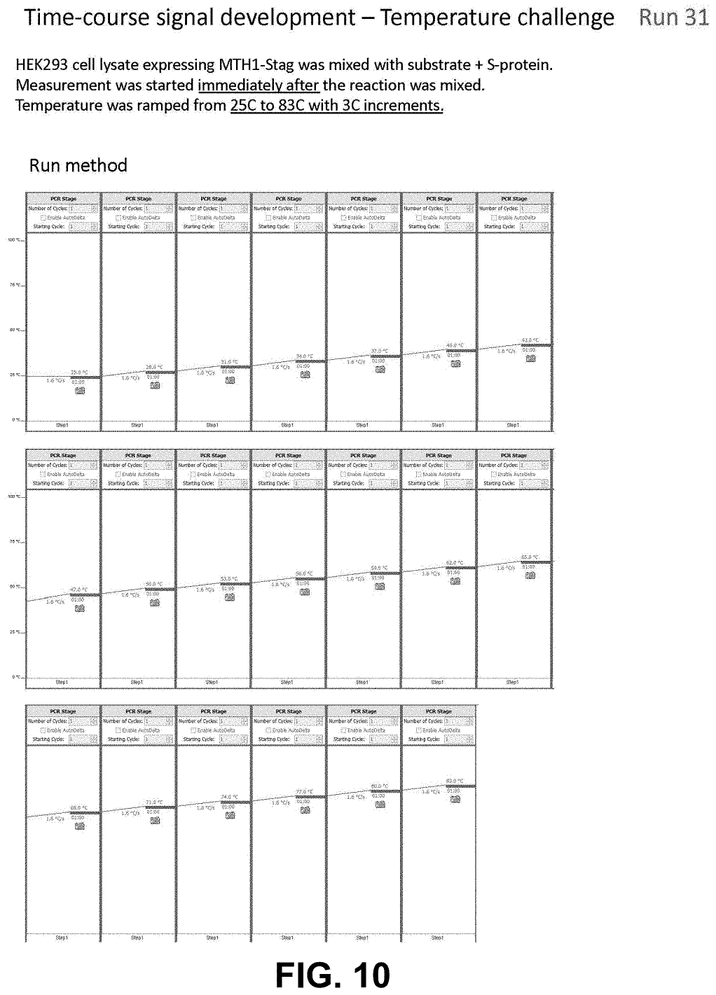

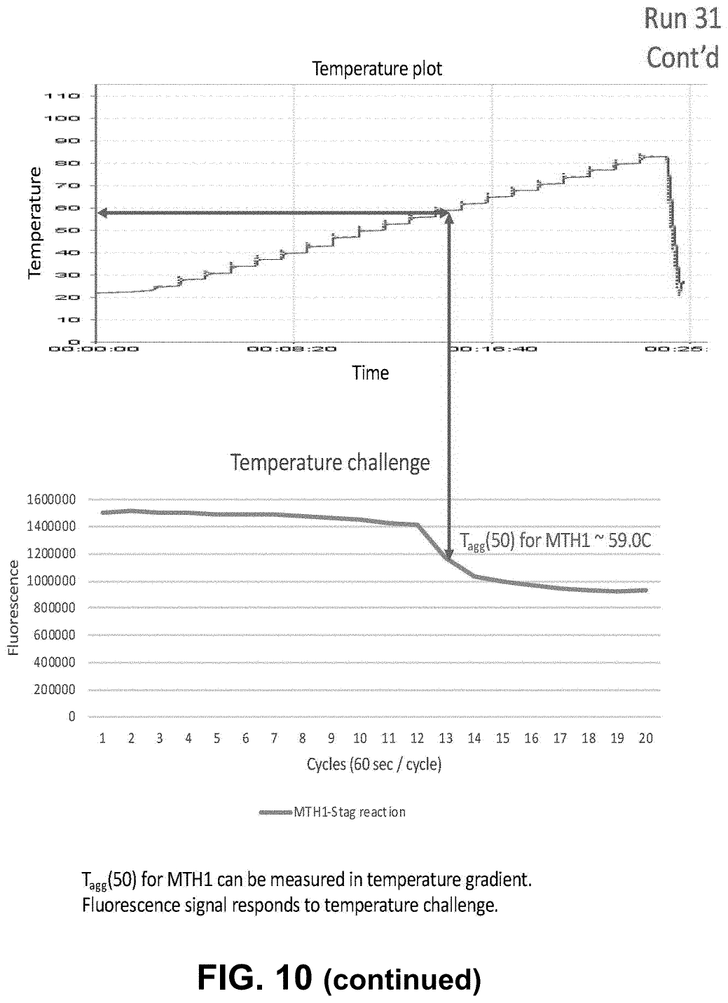

FIG. 10 and FIG. 10-Continued. Time-Course Signal Development—Temperature Challenge.

HEK293 cell were transfected with DNA encoding for MTH1-Stag fusion. 96-well RT-QPCR reaction plate was used to set up the target engagement reactions. HEK293 cells expressing MTH1-Stag fusion protein were lysed and complemented with nuclease substrate+S-protein. Measurement of fluorescence signal was started immediately in RT-QPCR machine after the reaction was mixed. Temperature was ramped from 2° C. to 83° C. with 3° C. increments.

Run in RT-QPCR machine was programmed as a 20-step reaction, each composed of multiple 60 sec cycles, such that in each step temperature is ramped with 3° C. increments, starting from 25° C. and reaching 83° C. (FIG. 10). Temperature plot (FIG. 10-continued, top) and fluorescence signal development in real time (FIG. 10-continued, bottom) are shown. Real-time signal development was measured over the course of 20 cycles (each 60 sec). Thermal melting profile is shown in terms of fluorescence signal for MTH1-Stag fusion protein in relation to temperature gradient. Temperature of aggregation 50 (T_{agg}50) can be calculated as 59° C. for the MTH1-Stag fusion protein. Fluorescence signal can be used to determine T_{agg}50.

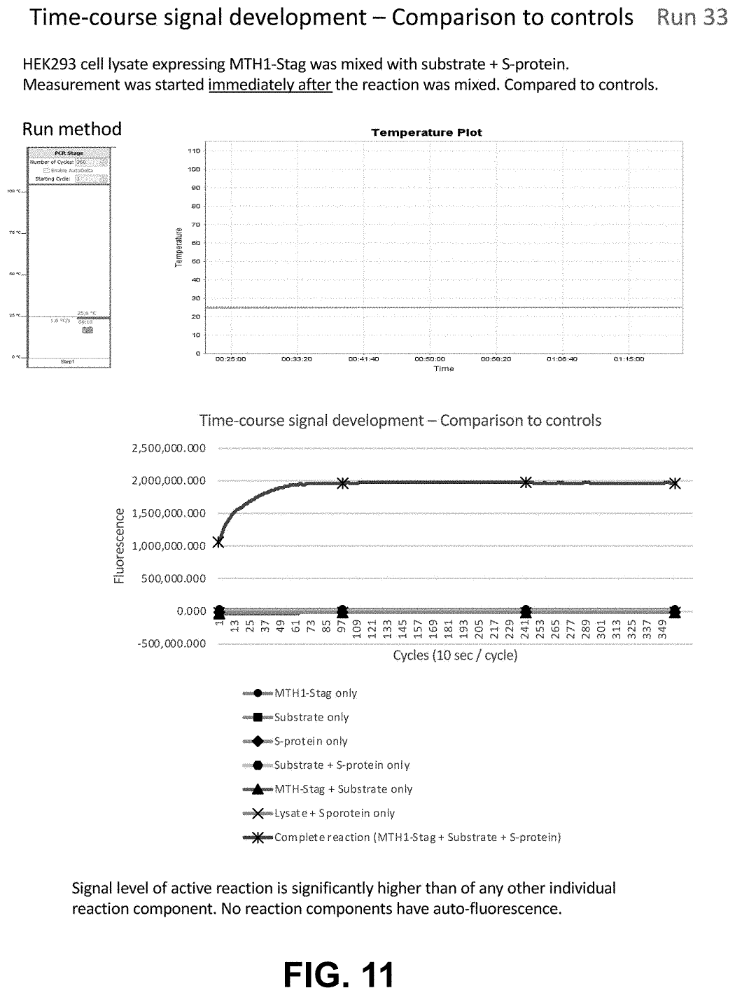

### FIG. 11. Time-Course Signal Development—Comparison to Controls.

HEK293 cell were transfected with DNA encoding for MTH1-Stag fusion. 96-well RT-QPCR reaction plate was used to set up the target engagement reactions. HEK293 cells expressing MTH1-Stag fusion protein were lysed and complemented with nuclease substrate+S-protein. Measurement of fluorescence signal was started immediately in RT-QPCR machine after the reaction was mixed. Signal of the complete reaction (MTH1_Stag expressing cell lysate+S protein+Substrate) was compared to several controls: MTH1-Stag only, Substrate only, S protein only, Substrate+S protein only, MTH1-Stag+Substrate only, Lysate+S protein only.

Run in RT-QPCR machine was programmed as a 1-step reaction composed of multiple 10 sec cycles, each at 25° C. (top left figure). Temperature plot (top right) and fluorescence signal development in real time (bottom right) are shown. Real-time signal development was measured over the course of 349 cycles (each 10 sec). Liner phase and plateau phase of signal development are observed for all reactions. Compared to all the controls, only the complete reaction (MTH1_Stag expressing cell lysate+S protein+Substrate) developed fluorescence signal in real time.

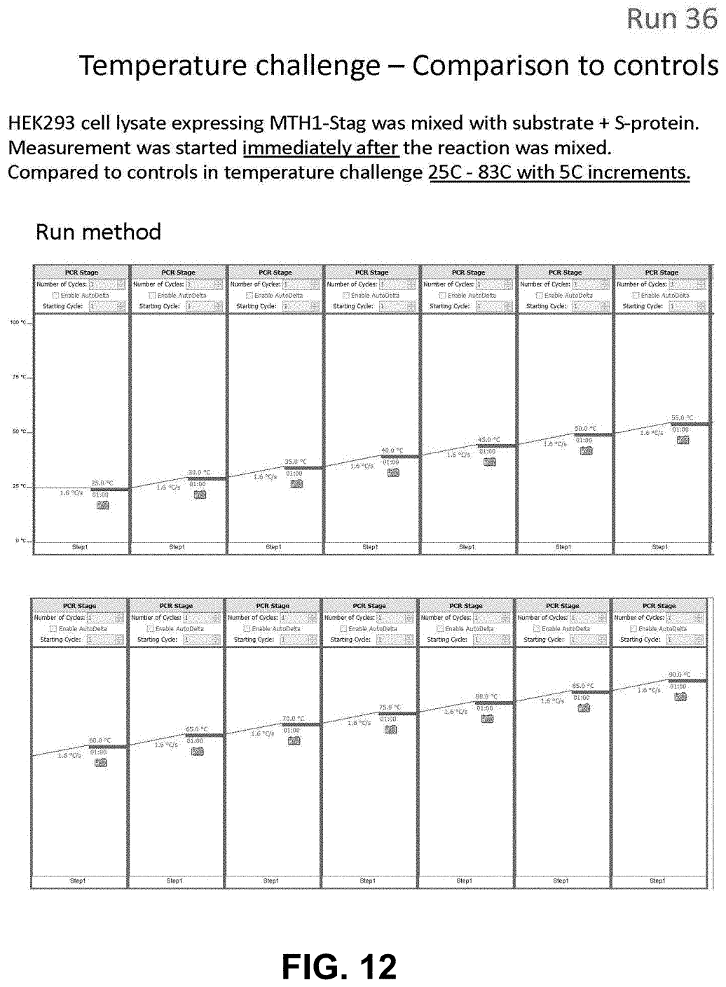

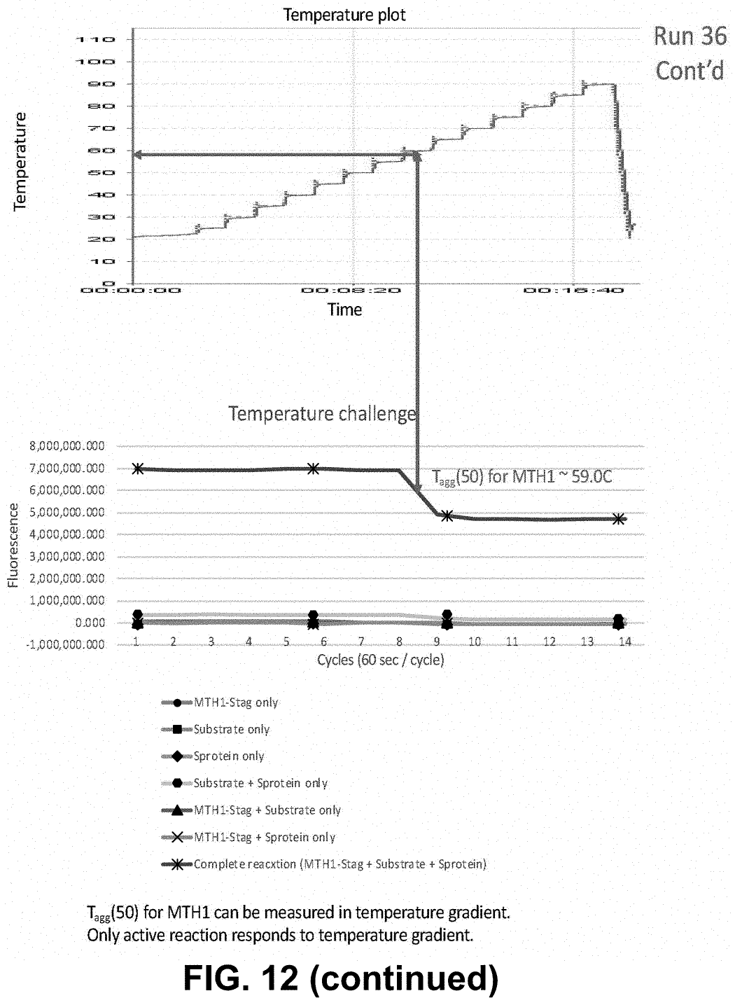

FIG. 12 and FIG. 12-Continued. Temperature Challenge—Comparison to Controls.

HEK293 cell were transfected with DNA encoding for MTH1-Stag fusion. 96-well RT-QPCR reaction plate was used to set up the target engagement reactions. HEK293 cells expressing MTH1-Stag fusion protein were lysed and complemented with nuclease substrate+S-protein. Measurement of fluorescence signal was started immediately in RT-QPCR machine after the reaction was mixed. Signal of the complete reaction (MTH1_Stag expressing cell lysate+S protein+Substrate) was compared to several controls: MTH1-Stag only, Substrate only, S protein only, Substrate+S protein only, MTH1-Stag+Substrate only, Lysate+S protein only.

Run in RT-QPCR machine was programmed as a 14-step reaction, each composed of multiple 60 sec cycles, such that in each step temperature is ramped with 5° C. increments, starting from 25° C. and reaching 83° C. (FIG. 12). Temperature plot (FIG. 12-continued, top) and fluorescence signal development in real time (FIG. 12-continued, bottom) are shown. Real-time signal development was measured over the course of 14 cycles (each 60 sec). Thermal melting profile is shown in terms of fluorescence signal for MTH1-Stag fusion protein in relation to temperature gradient. Compared to all the controls, only the complete reaction (MTH1_Stag expressing cell lysate+S protein+Substrate) developed temperature-dependent fluorescence signal in real time. Temperature of aggregation 50 (T_{agg}50) can be calculated as 59° C. for the MTH1-Stag fusion protein.

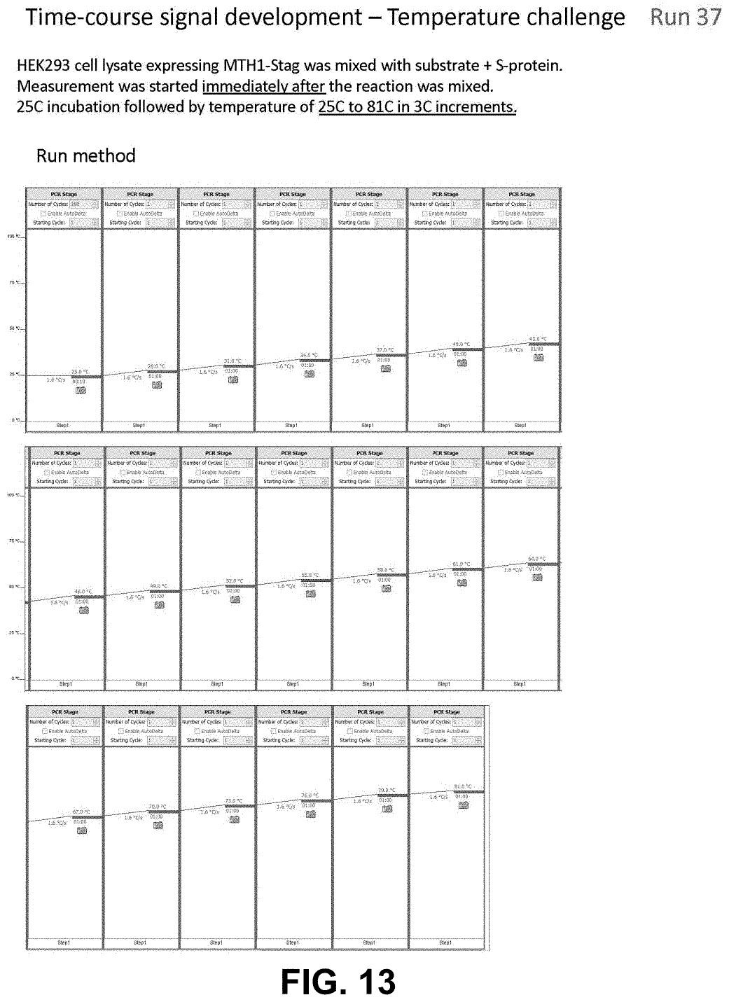

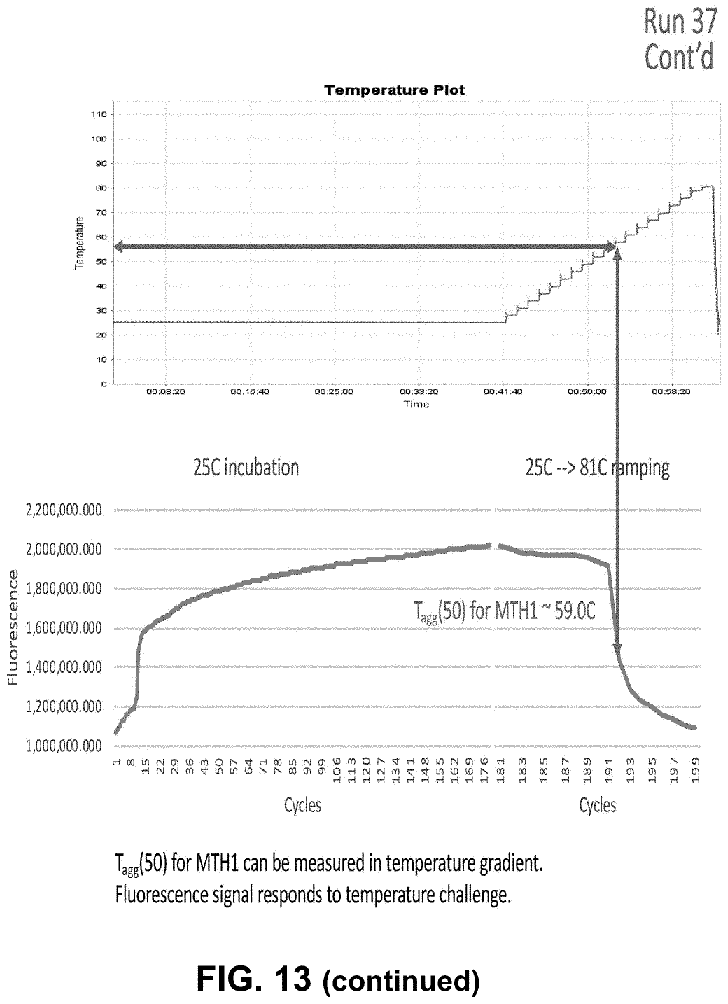

FIG. 13 and FIG. 13-Continued. Temperature Challenge—Comparison to Controls.

HEK293 cell were transfected with DNA encoding for MTH1-Stag fusion. 96-well RT-QPCR reaction plate was used to set up the target engagement reactions. HEK293 cells expressing MTH1-Stag fusion protein were lysed and complemented with nuclease substrate+S-protein. Measurement of fluorescence signal was started immediately in RT-QPCR machine after the reaction was mixed.

Run in RT-QPCR machine was programmed as a 20-step reaction, each composed of multiple 60 sec cycles, such that a 180-cycle incubation at 25° C. is followed by 1-cycle temperature ramping with 3° C. increments, starting from 25° C. and reaching 83° C. (FIG. 13). Temperature plot (FIG. 13-continued, top) and fluorescence signal development in real time (FIG. 13-continued, bottom) are shown. Thermal melting profile is shown in terms of fluorescence signal for MTH1-Stag fusion protein in relation to temperature gradient in real time. Temperature of aggregation 50 (T_{agg}50) can be calculated as 59 C for the MTH1-Stag fusion protein. This figure demonstrates that it is possible to program the real-time target engagement reaction to measure signal maturity and thermal profile of the target T_{agg}(50) in response to temperature challenge in one setting.

FIG. 14. FIG. 14-Continued-1 and FIG. 14-Continued-2—Time-Course Signal Development—Temperature Challenge with Crizotinib Dose Range.

HEK293 cell were transfected with DNA encoding for MTH1-Stag fusion. 384-well RT-QPCR reaction plate was used to set up the target engagement reactions. HEK293 cells expressing MTH1-Stag fusion protein were treated with Crizotinib in dose range of 10.000, 2000, 400, 80, 16, 3.2, 0.64, 0 nM for 1 hr. The cells were then lysed and complemented with nuclease substrate+S-protein. Measurement of fluorescence signal was started immediately in RT-QPCR machine after the reaction was mixed.

Run in RT-QPCR machine was programmed as a 19-step reaction multiplexing reaction where various doses of the Crizotinib were independently tested at temperature gradient. Each step was composed of multiple 10 sec cycles, such that a 360-cycle incubation at 25° C. is followed by 1-cycle temperature ramping with 3° C. increments, starting from 25° C. and reaching 81° C. (FIG. 14). Temperature plot (FIG. 14-continued-1, top) and fluorescence signal development in real time (FIG. 14-continued-1, bottom) are shown for the 25° C. incubation phase.

Shown in FIG. 14-continued-2 is correlation of the temperature plot with real-time fluorescence/thermal profile of MTH1-Stag treated with various doses of Crizotinib during the temperature ramping phase. Temperature of aggregation 50 (T_{agg}50) can be calculated as 59° C. for the MTH1-Stag fusion protein. Additionally, the dose-dependent effect of Crizotinib on T_{agg}50 can be observed and measured. This figure demonstrates that it is possible to program multiplexed real-time target engagement reaction to measure Temperature of aggregation of the target in relation to dose of the inhibitor. This type of multiplexed reaction will generate multi-dimensional cell target engagement data.

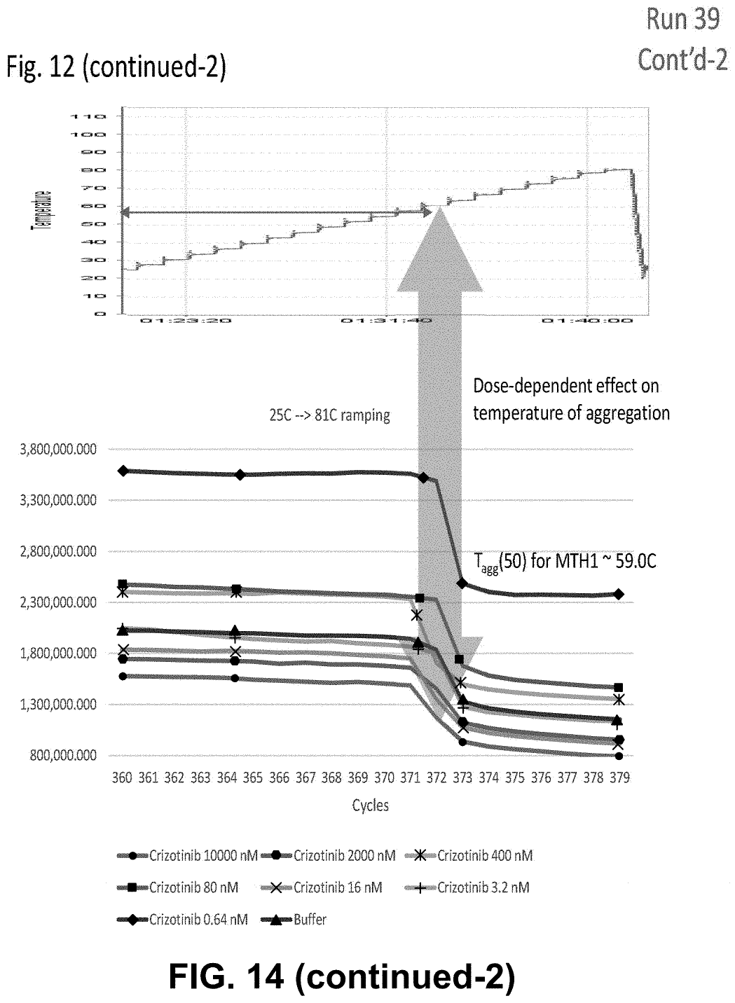

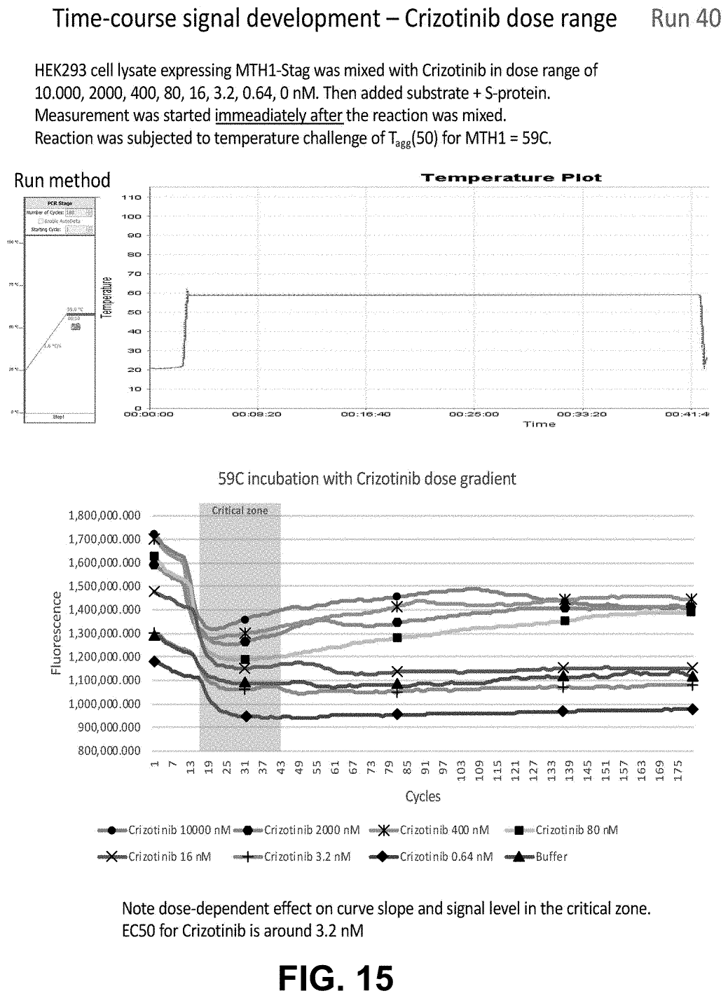

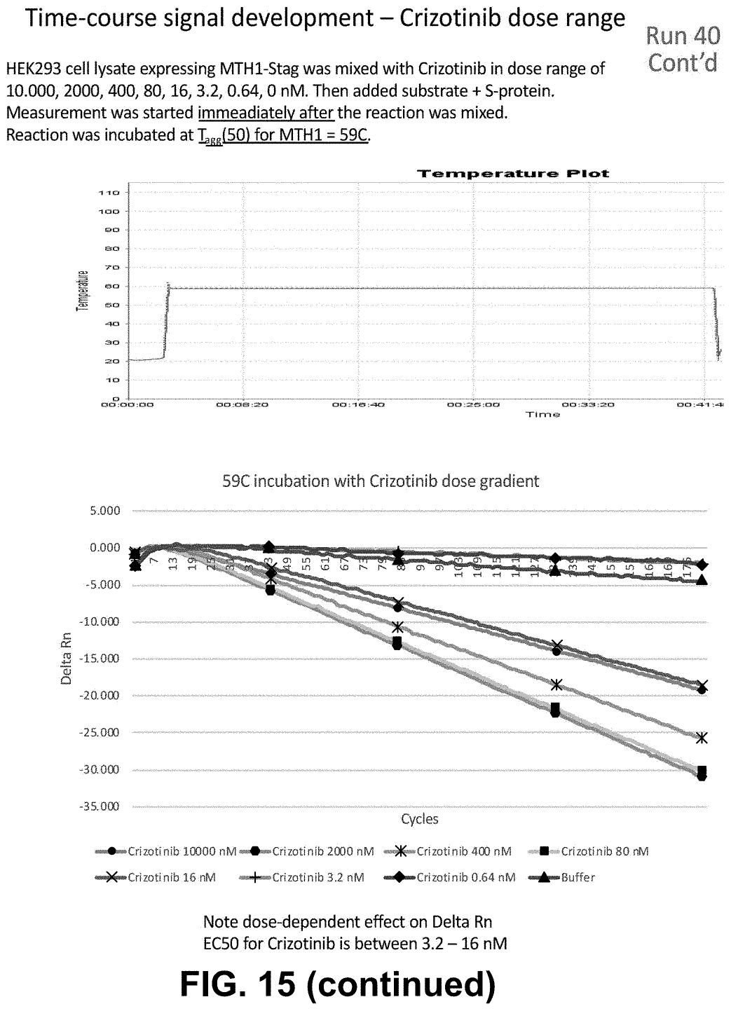

### FIG. 15, FIG. 15-Continued—Time-Course Signal Development—Crizotinib Dose Range

HEK293 cell were transfected with DNA encoding for MTH1-Stag fusion. 96-well RT-QPCR reaction plate was used to set up the target engagement reactions. HEK293 cells expressing MTH1-Stag fusion protein were treated with Crizotinib in dose range of 10.000, 2000, 400, 80, 16, 3.2, 0.64, 0 nM for 1 hr. The cells were then lysed and complemented with nuclease substrate+S-protein. Measurement of fluorescence signal was started immediately in RT-QPCR machine after the reaction was mixed.

Run in RT-QPCR machine was programmed as a 1-step reaction where various doses of the Crizotinib were tested at stable temperature point of 59° C. The 1-step reaction step was composed of 180×10-sec cycles, temperature was ramped immediately to 59° C. (FIG. 15, top left). Temperature plot (FIG. 15, top right) and fluorescence signal development in real time (FIG. 15, bottom right) are shown for the 59° C. temperature challenge phase.

Shown in FIG. 15 is correlation of the 59° C. temperature challenge with real-time fluorescence/thermal profile of MTH1-Stag treated with various doses of Crizotinib. Note the dose-dependent effect on curve slope and signal level in the critical zone, enabling calculation of EC50 for Crizotinib around 3.2 nM.

Delta-Rn can serve as another read-out for dose-dependent fluorescence signal. Shown in FIG. 15-continued is correlation of the 59° C. temperature challenge with real-time Delta-Rn/thermal profile of MTH1-Stag treated with various doses of Crizotinib. Note the dose-dependent effect on Delta-Rn, enabling calculation of EC50 for Crizotinib between 3.2-16 nM. This type of reaction will generate multi-dimensional cell target engagement data.

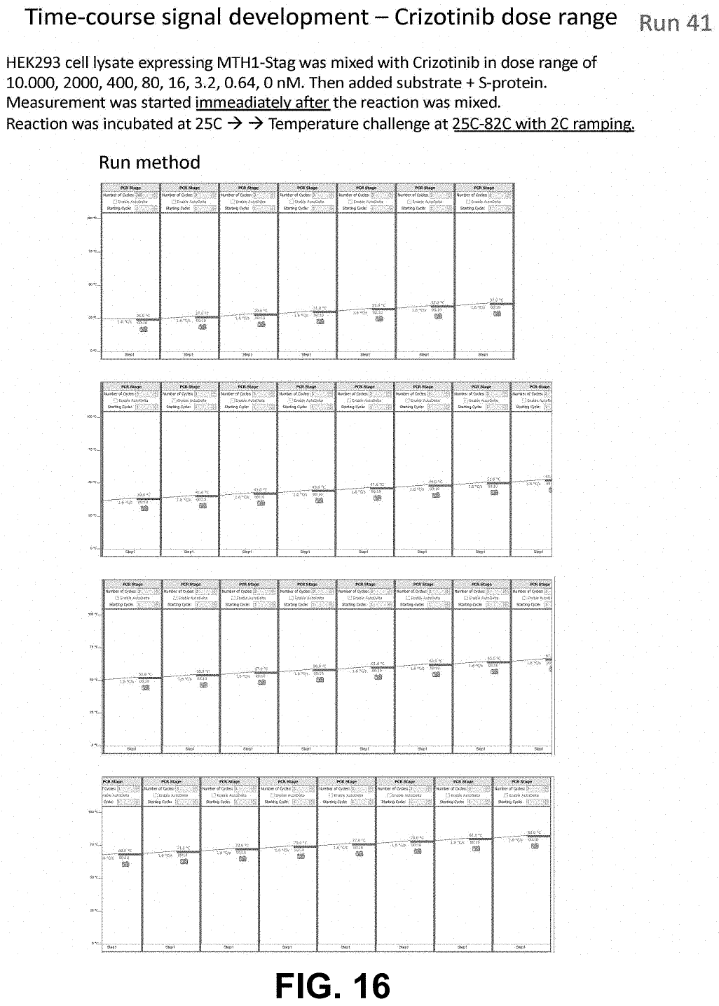

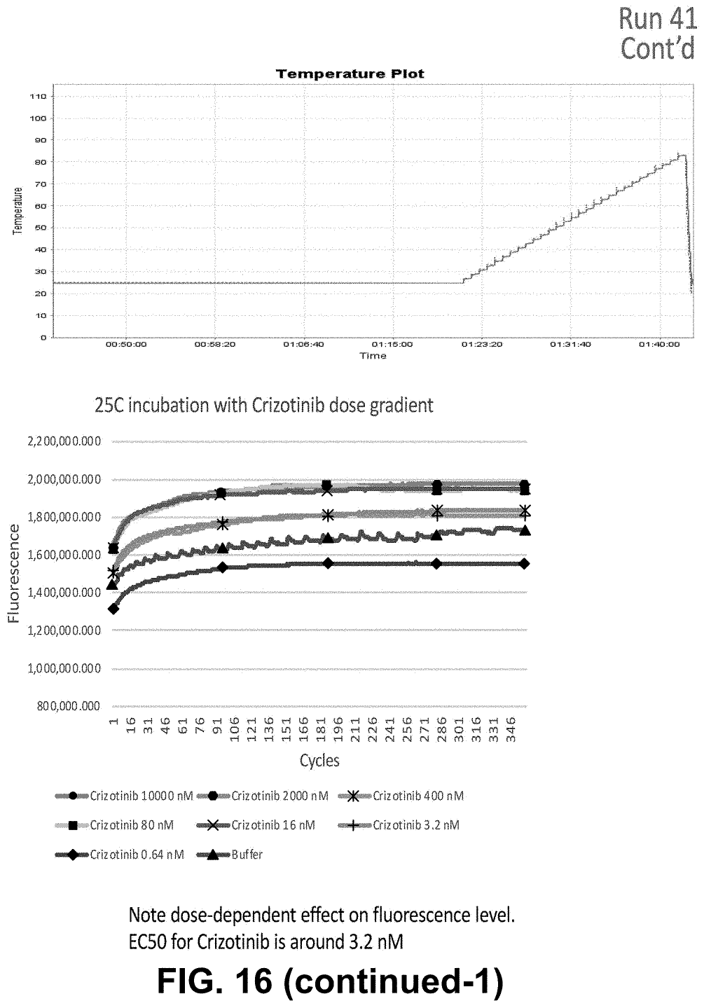

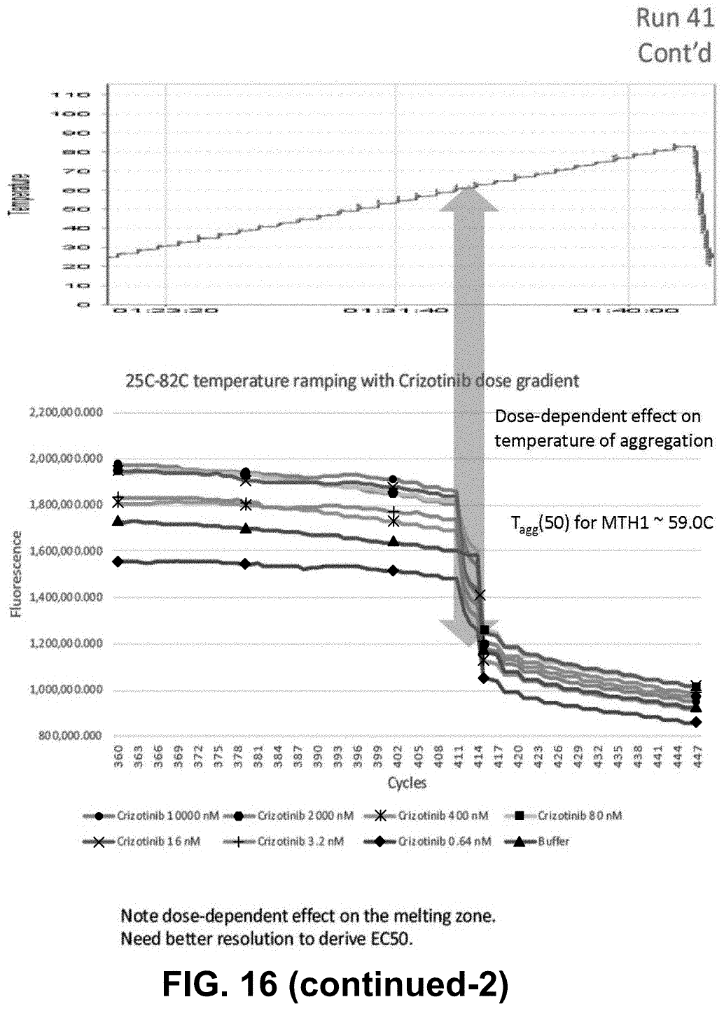

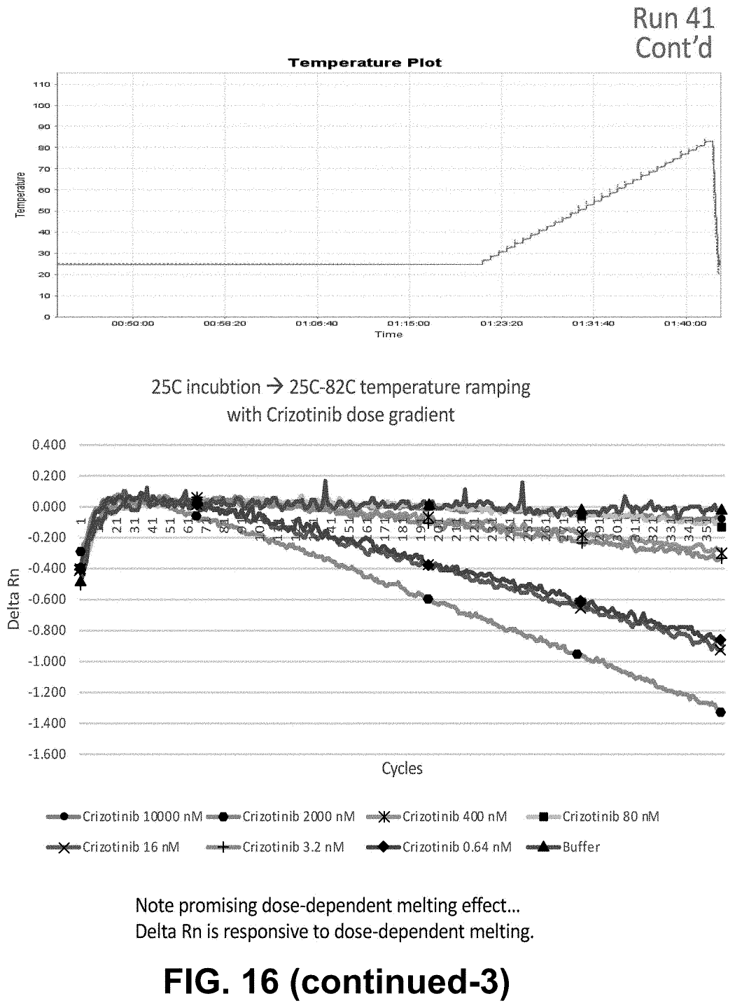

### FIG. 16, FIG. 16-Continued-1. FIG. 16-Continued-2. FIG. 16-Continued-3—Time-Course Signal Development—Crizotinib Dose Range

HEK293 cell were transfected with DNA encoding for MTH1-Stag fusion. 96-well RT-QPCR reaction plate was used to set up the target engagement reactions. HEK293 cells expressing MTH1-Stag fusion protein were treated with Crizotinib in dose range of 10.000, 2000, 400, 80, 16, 3.2, 0.64, 0 nM for 1 hr. The cells were then lysed and complemented with nuclease substrate+S-protein. Measurement of fluorescence signal was started immediately in RT-QPCR machine after the reaction was mixed with a 25° C. incubation followed by a 25° C.-82° C. temperature challenge.

Run in RT-QPCR machine was programmed as a 23-step reaction where various doses of the Crizotinib were independently tested in temperature gradient. Each step was composed of multiple 10 sec cycles, such that a 360-cycle incubation at 25° C. is followed by 3-cycle temperature ramping with 2° C. increments, starting from 25° C. and reaching 82° C. (FIG. 16). Temperature plot (FIG. 16-continued-1, top) and fluorescence signal development in real time (FIG. 16-continued-1, bottom) are shown for the 25° C. incubation phase.

Shown in FIG. 16-continued-2 is correlation of the temperature plot with real-time fluorescence/thermal profile of MTH1-Stag treated with various doses of Crizotinib during the temperature ramping phase. Temperature of aggregation 50 (T_{agg}50) can be calculated as 59° C. for the MTH1-Stag fusion protein. Additionally, the dose-dependent effect of Crizotinib on T_{agg}50 of the target can be observed and measured in a single run.

Shown in FIG. 16-continued-3 is correlation of the temperature plot Delta-Rn/thermal profile of MTH1-Stag treated with various doses of Crizotinib during the 25° C. incubation and temperature ramping phases. Temperature of aggregation 50 (T_{agg}50) can be calculated for the MTH1-Stag fusion protein. Additionally, the dose-dependent effect of Crizotinib on T_{agg}50 of the target can be observed and measured in a single run with Delta-Rn as readout.

FIG. 16 demonstrates that it is possible to program real-time target engagement reaction to measure Temperature of aggregation of the target in relation to dose of the inhibitor. This type of reaction will generate multi-dimensional cell target engagement data.

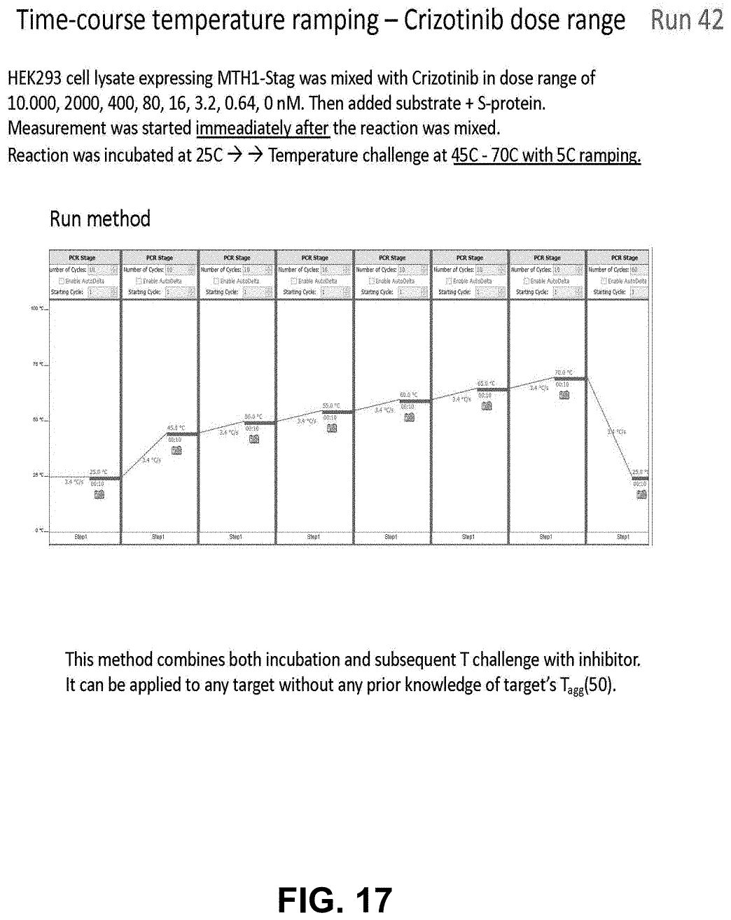

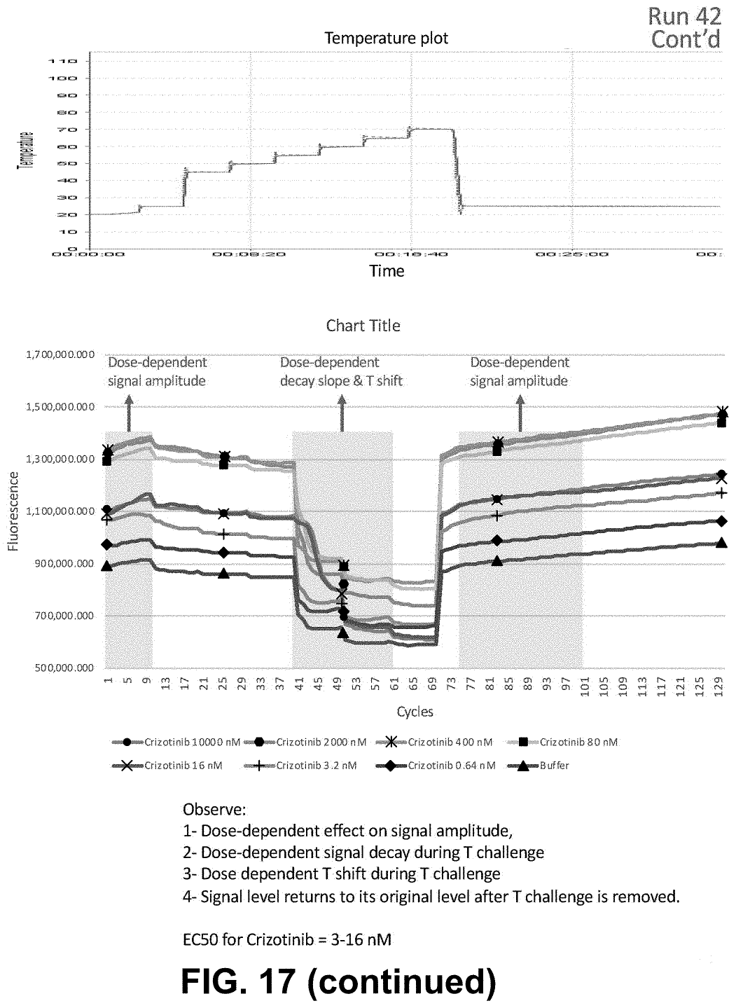

### FIG. 17, FIG. 17-Continued—Time-Course Temperature Ramping—Crizotinib Dose Range

HEK293 cell were transfected with DNA encoding for MTH1-Stag fusion. 96-well RT-QPCR reaction plate was used to set up the target engagement reactions. HEK293 cells expressing MTH1-Stag fusion protein were treated with Crizotinib in dose range of 10.000, 2000, 400, 80, 16, 3.2, 0.64, 0 nM for 1 hr. The cells were then lysed and complemented with nuclease substrate+S-protein. Measurement of fluorescence signal was started immediately in RT-QPCR machine after the reaction was mixed, with a 25° C. incubation followed by a 45° C.-70° C. temperature challenge with 5° C. temperature increments.

Run in RT-QPCR machine was programmed as a 8-step reaction where various doses of the Crizotinib were independently tested in temperature gradient. Each step was composed of multiple 10 sec cycles, such that a 10-cycle incubation at 25° C. is followed by 10-cycle temperature ramping with 5° C. increments, starting from 45° C. and reaching 70° C., then going back to 60-cycle incubation at 25° C. (FIG. 17). This method combines both incubation and subsequent T challenge with inhibitor. It can be applied to any target without any prior knowledge of target's T_{agg}(50), all combined in one run.

Temperature plot (FIG. 17-continued, top) and fluorescence signal development in real time (FIG. 17-continued, bottom) are shown for the entire run. Note that the real-time fluorescence profile of the target reveals at least three important zones: Dose-dependent signal amplitude zone 1 (cycles 1-9); Dose-dependent decay slope and T shift zone (cycles 40-61); and Dose-dependent signal amplitude zone 2 (cycles 75-100).

In the Dose-dependent signal amplitude zone 1 (cycles 1-9), note the dose-dependent effect on curve slope and signal level, enabling calculation of EC50 for Crizotinib. In the Dose-dependent decay slope and T shift zone (cycles 40-61), note the dose-dependent effect on curve decay, enabling independent calculation of EC50 for Crizotinib. In the Dose-dependent signal amplitude zone 2 (cycles 75-100), note regeneration of signal at 25° C. with dose-dependent curve slope and signal level, enabling independent calculation of EC50 for Crizotinib.

This run enables calculation of these key dimensions of cell target engagement: 1) Dose-dependent effect on fluorescence signal amplitude; 2) Dose-dependent fluorescence signal decay during temperature challenge; 3) Dose dependent shift in temperature of aggregation (T_{agg}) during temperature challenge; 4) Regeneration of fluorescence signal level to after temperature challenge is removed; 5) EC50 for Crizotinib was calculated between 3-16 nM. This type of programmed reaction will generate multi-dimensional cell target engagement data.

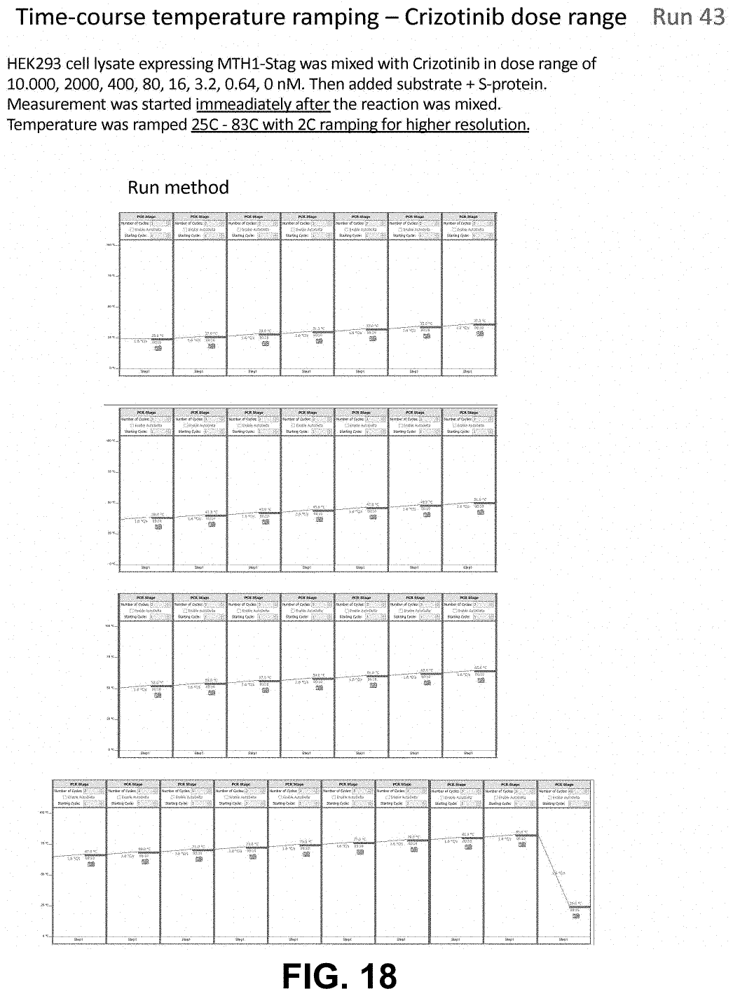

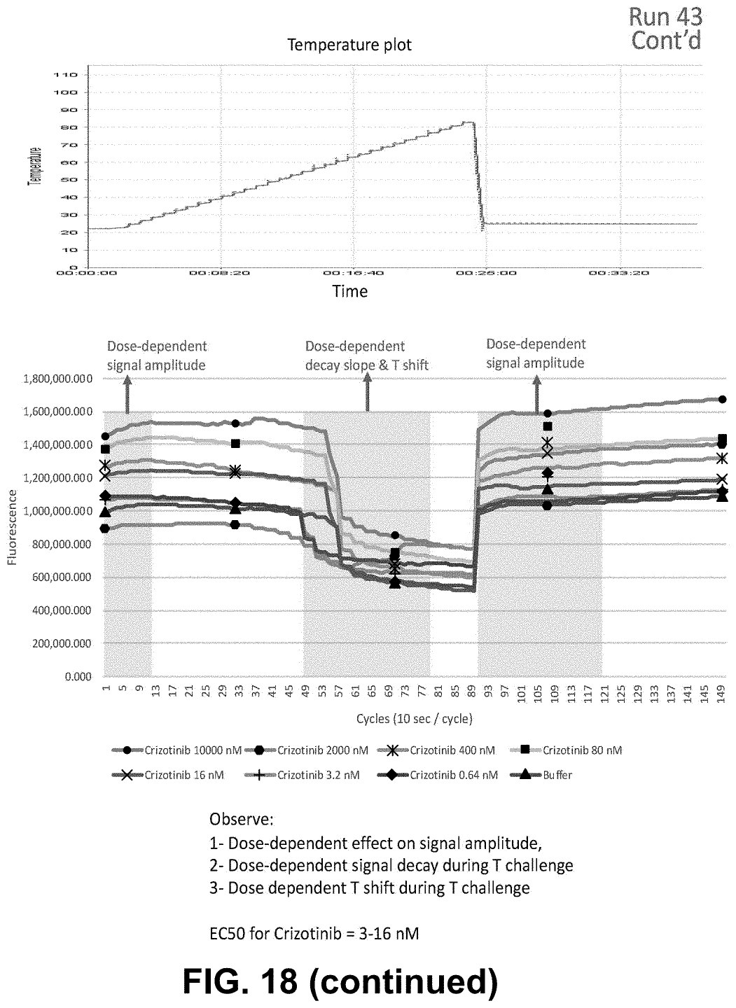

### FIG. 18, FIG. 18-Continued—Time-Course Temperature Ramping—Crizotinib Dose Range

HEK293 cell were transfected with DNA encoding for MTH1-Stag fusion. 96-well RT-QPCR reaction plate was used to set up the target engagement reactions. HEK293 cells expressing MTH1-Stag fusion protein were treated with Crizotinib in dose range of 10.000, 2000, 400, 80, 16, 3.2, 0.64, 0 nM for 1 hr. The cells were then lysed and complemented with nuclease substrate+S-protein. Measurement of fluorescence signal was started immediately in RT-QPCR machine after the reaction was mixed, then, temperature was ramped 25° C.-83° C. with 2° C. ramping for signal high resolution in a single run.

Run in RT-QPCR machine was programmed as a 31-step reaction where various doses of the Crizotinib were independently tested in temperature gradient. Each step was composed of multiple 10 sec cycles, such that a 3-cycle incubation at 25° C. is followed by 3-cycle temperature ramping with 2° C. increments, reaching 83° C., then going back to 60-cycle incubation at 25° C. (FIG. 18). This method combines both incubation and subsequent T challenge with inhibitor. It can be applied to any target without any prior knowledge of target's T_{agg}(50), all combined in one run, thus generating high-resolution target engagement profile.

Temperature plot (FIG. 18-continued, top) and fluorescence signal development in real time (FIG. 18-continued, bottom) are shown for the entire run. Note that the real-time fluorescence profile of the target reveals at least three important zones: Dose-dependent signal amplitude zone 1 (cycles 1-49); Dose-dependent decay slope and T shift zone (cycles 49-80); and Dose-dependent signal amplitude zone 2 (cycles 90-120).

In the Dose-dependent signal amplitude zone 1 (cycles 1-49), note the dose-dependent effect on curve slope and signal level, enabling calculation of EC50 for Crizotinib. In the Dose-dependent decay slope and T shift zone (cycles 49-80), note the dose-dependent effect on curve decay, enabling independent calculation of EC50 for Crizotinib. In the Dose-dependent signal amplitude zone 2 (cycles 90-120), note regeneration of signal at 25° C. with dose-dependent curve slope and signal level, enabling independent calculation of EC50 for Crizotinib.

This run enables calculation of these key dimensions of cell target engagement: 1) Dose-dependent effect on fluorescence signal amplitude; 2) Dose-dependent fluorescence signal decay during temperature challenge; 3) Dose dependent shift in temperature of aggregation (T_{agg}) during temperature challenge; 4) Regeneration of fluorescence signal level to after temperature challenge is removed; 5) EC50 for Crizotinib was calculated between 3-16 nM. This type of programmed reaction will generate high-resolution multi-dimensional cell target engagement data.

FIG. 19. Monitoring Target Engagement of Crizotinib Enantiomers to MTH1 Stag Fusion with QuantStudio 3 Real-Time qPCR System.

For the experiments shown on panel A of FIG. 19, both the (S) and (R) enantiomers of Crizotininb were added to lysates from cells expressing MTH1 Stag fusion. Samples were heated at 54° C. as determined from the thermal profile for MTH1 in FIG. 2, followed by enzyme complementation assay and fluorescence detection with QuantStudio 3 real-time qPCR system. In panel B of FIG. 19, the fluorescence from the linear part of the curves from panel A (cycle 10; 10 min) was plotted with dose of inhibitor on a semi-log graph, and non-linear regression analysis was used to fit a curve using Log agonist vs. variable response four parameter logistic (4PL) regression.

### Example 4

Returning to FIG. 2, system 10 is configured for real-time or near real time cellular drug-target engagement. System 10 can be used for determining if a test compound can interact with a target polypeptide, for example. In some embodiments, system 10 comprises a processor 14, a machine 22, a server 26, a data store 30, a mobile user device 34, a desktop user device 38, external resources 46, a network 50, and/or other components. Each of these components is described, in turn, below.

Processor 14 is configured to provide information-processing capabilities in system 10. As such, processor 14 may comprise one or more of a digital processor, an analog processor, a digital circuit designed to process information, an analog circuit designed to process information, a state machine, and/or other mechanisms for electronically processing information. Although processor 14 is shown in FIG. 2 as a single entity, this is for illustrative purposes only. In some embodiments, processor 14 may comprise a plurality of processing units. These processing units may be physically located within the same device (e.g., within machine 22, server 26, mobile user device 34, desktop user device 38, etc.), or processor 14 may represent processing functionality of a plurality of devices operating in coordination. In some embodiments, processor 14 may be and/or be included in a computing device such as a desktop computer, a laptop computer, a smartphone, a tablet computer, a server, and/or other computing devices. These computing devices may run one or more electronic applications having graphical user interfaces configured to facilitate user interaction with system 10. In some embodiments, processor 14 may be included in and/or control machine tool 22, for example.

Processor 14 is configured by machine readable instructions 15 to execute one or more computer program components. The computer program components may comprise software programs and/or algorithms coded and/or otherwise defined by machine readable instructions 15 and/or embedded in processor 14, for example. Processor 14 may be configured to execute the computer program components by software; hardware; firmware; some combination of software, hardware, and/or firmware; and/or other mechanisms for configuring processing capabilities on processor 14.

Machine 22 is configured to function as described above.

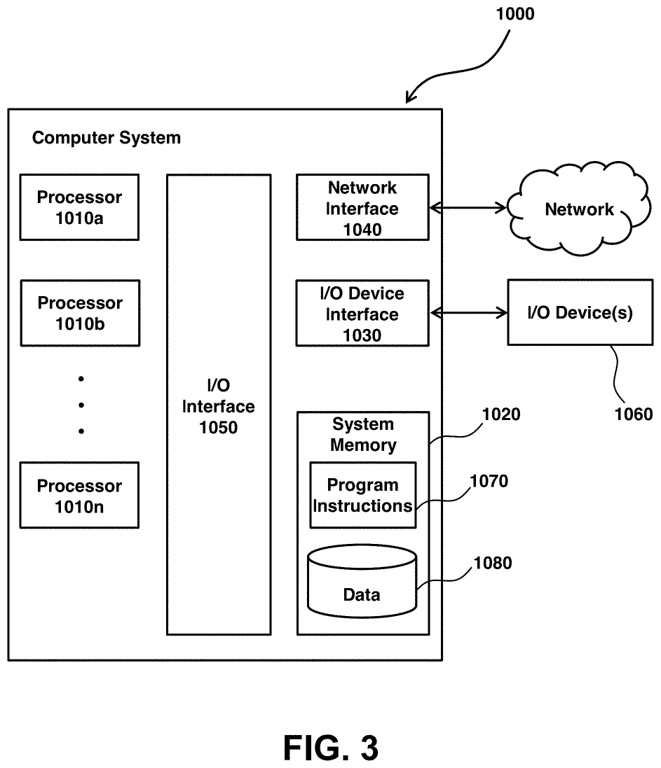

In some embodiments, processor 14 is executed by one or more of the computers described below with reference to FIG. 3. The components of system 10, in some embodiments, communicate with one another in order to provide the functionality of processor 14, machine 22, and/or other components described herein. In some embodiments, data store 30 may store data about one or more of the operations described above, or other information. Server 26 may expedite access to this data by storing likely relevant data in relatively high-speed memory, for example, in random-access memory or a solid-state drive. Server 26 may communicate with webpages and/or other sources of network information. Server 26 may serve data to various applications that process data related to the operations described above, and/or other data. The operation of server 26 and data store 30 may be coordinated by one or more processors 14 (which may be located within and/or formed by machine 22, server 26, mobile user device 34, desktop user device 38, external resources 46, and/or other computing devices), which may bidirectionally communicate with each of these components or direct the components to communicate with one another. Communication may occur by transmitting data between separate computing devices (e.g., via transmission control protocol/internet protocol (TCP/IP) communication over a network), by transmitting data between separate applications or processes on one computing device; or by passing values to and from functions, modules, or objects within an application or process, e.g., by reference or by value.

In some embodiments, interaction with users (e.g., sending and/or receiving requests for information, etc.) may be facilitated by processor 14, machine 22, server 26, mobile user device 34, desktop user device 38, and/or other components. This may occur via a user interface or a native application viewed on machine 22, a desktop computer (e.g., desktop user device 38), a mobile computer (e.g., mobile user device 34) such as a tablet, or a laptop of the user. In some embodiments, such interaction occurs via a mobile website viewed on a smart phone, tablet, or other mobile user device, or via a special-purpose native application executing on a smart phone, tablet, or other mobile user device.

To illustrate an example of the environment in which processor 14 operates, the illustrated embodiment of FIG. 2 includes a number of components with which processor 14 communicates: machine 22; server 26; data store 30; mobile user device(s) 34; a desktop user device 38; and external resources 46. These devices communicate with processor 14 via a network 50, such as the Internet or the Internet in combination with various other networks, like local area networks, cellular networks, or personal area networks, internal organizational networks, and/or other networks.

Mobile user device(s) 34 may be smart phones, tablets, or other hand-held networked computing devices having a display, a user input device (e.g., buttons, keys, voice recognition, or a single or multi-touch touchscreen), memory (such as a tangible, machine-readable, non-transitory memory), a network interface, a portable energy source (e.g., a battery), and a processor (a term which, as used herein, includes one or more processors) coupled to each of these components. The memory of mobile user device(s) 34 may store instructions that when executed by the associated processor provide an operating system and various applications, including a web browser and/or a native mobile application.

Desktop user device(s) 38 may also include a web browser, a native application, and/or other components. In addition, desktop user device(s) 38 may include a monitor; a keyboard; a mouse; memory; a processor; and a tangible, non-transitory, machine-readable memory storing instructions that when executed by the processor provide an operating system, the web browser, the native application, and/or other components. Native applications and web browsers, in some embodiments, are operative to provide a graphical user interface that communicates with processor 14 and facilitates user interaction with data from processor 14. Web browsers may be configured to receive a website and/or other web based communications from processor 14 having data related to instructions (for example, instructions expressed in JavaScript™) that when executed by the browser (which is executed by a processor) cause mobile user device 34 and/or desktop user device 38 to communicate with processor 14 and facilitate user interaction with data from processor 14. Native applications and web browsers, upon rendering a webpage and/or a graphical user interface from processor 14, may generally be referred to as client applications of processor 14 (and/or server 26, which may include processor 14), which in some embodiments may be referred to as a server. Embodiments, however, are not limited to client/server architectures, and processor 14, as illustrated, may include a variety of components other than those functioning primarily as a server.

In some embodiments, machine 22 may include one or more computing components configured to perform one or more of the operations associated with mobile user device 34 and/or desktop user device 38 described above, and/or may include desktop user device 38 itself, for example.

External resources 46, in some embodiments, include sources of information such as databases, websites, etc.; external entities participating with system 10 (e.g., systems or networks that store data related to one or more of the operations described above; one or more servers outside of the system 10; a network (e.g., the internet); electronic storage; equipment related to Wi-Fi™ technology; equipment related to Bluetooth® technology; data entry devices; or other resources. In some embodiments, some or all of the functionality attributed herein to external resources 46 may be provided by resources included in system 10. External resources 46 may be configured to communicate with processor 14, machine 22, server 26, mobile user devices 34, desktop user devices 38, and/or other components of system 10 via wired and/or wireless connections, via a network (e.g., a local area network and/or the internet), via cellular technology, via Wi-Fi technology, and/or via other resources. The number of illustrated processors 14, machines 22, external resources 46, servers 26, desktop user devices 38, and mobile user devices 34 is selected for explanatory purposes only, and embodiments are not limited to the specific number of any such devices illustrated by FIG. 2, which is not to imply that other descriptions are limiting.

System 10 includes a number of components introduced above that facilitate requests for the processing operations described herein by users, other computing systems, and/or requests from other sources. For example, server 26 may be configured to communicate data about requests, results of those requests, and/or other information via a protocol, such as a representational-state-transfer (REST)-based API protocol over hypertext transfer protocol (HTTP) or other protocols. Examples of operations that may be facilitated by server 26 include requests to display, link, modify, add, or retrieve portions of an electronic model of a metallic part, and/or results of such requests, or other information. API requests may identify which data is to be displayed, linked, modified, added, or retrieved by specifying criteria for identifying records, such as queries for retrieving or processing information about a particular metallic part. In some embodiments, server 26 communicates with the native applications of machine 22, mobile user device 34 and desktop user device 38, and/or other components of system 10 (e.g., e.g., to send and/or receive such requests).

Server 26 may be configured to display, link, modify, add, or retrieve portions or all data related a particular operation, results from a particular operation, and/or other information encoded in a webpage (e.g. a collection of resources to be rendered by the browser and associated plug-ins, including execution of scripts, such as JavaScript™, invoked by the webpage), or in a graphical user interface display, for example. In some embodiments, a graphical user interface presented by the webpage may include inputs by which the user may enter or select data, such as clickable or touchable display regions or display regions for text input. Such inputs may prompt the browser to request additional data from server 26 or transmit data to server 26, and server 26 may respond to such requests by obtaining the requested data and returning it to the user device or acting upon the transmitted data (e.g., storing posted data or executing posted commands). In some embodiments, the requests are for a new webpage or for data upon which client-side scripts will base changes in the webpage, such as XMLHttpRequest requests for data in a serialized format, e.g. JavaScript™ object notation (JSON) or extensible markup language (XML). Server 26 may communicate with web browsers executed by user devices 34 or 38, a native application run by machine 22, and/or other components, for example. In some embodiments, a webpage is modified by server 26 based on the type of user device, e.g., with a mobile webpage having fewer and smaller images and a narrower width being presented to the mobile user device 34, and a larger, more content rich webpage being presented to machine 22, and/or desktop user device 38. An identifier of the type of user device, either mobile or non-mobile, for example, may be encoded in the request for the webpage by the web browser (e.g., as a user agent type in an HTTP header associated with a GET request), and server 26 may select the appropriate interface based on this embedded identifier, thereby providing an interface appropriately configured for the specific user device in use.

Data store 30 stores data related to the operations described above, requests for such operations, results from such requests, etc. Data store 30 may include various types of data stores, including relational or non-relational databases, document collections, hierarchical key-value pairs, or memory images, for example. Such components may be formed in a single database, document, or other component, or may be stored in separate data structures. In some embodiments, data store 30 comprises electronic storage media that electronically stores information. The electronic storage media of data store 30 may include one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Data store 30 may be (in whole or in part) a separate component within system 10, or data store 30 may be provided (in whole or in part) integrally with one or more other components of the system 10 (e.g., processors 14, etc.). In some embodiments, data store 30 may be located in a data center, in machine 22, in server 26, in a server that is part of external resources 46, in a computing device 34 or 38, or in other locations. Data store 30 may include one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), or other electronically readable storage media. Data store 30 may store software algorithms, information determined by processor 14, information received via a graphical user interface displayed on machine 22 and/or computing devices 34 and/or 38, information received from external resources 46, or other information accessed by the system 10 to function as described herein.

FIG. 3 is a diagram that illustrates an exemplary computing system 1000 in accordance with embodiments of the present system. Various portions of systems and methods described herein, may include or be executed on one or more computer systems the same as or similar to computing system 1000. For example, processor 14, machine 22, server 26, mobile user device 34, desktop user device 38, external resources 46 and/or other components of system 10 (FIG. 2) may be and/or include one more computer systems the same as or similar to computing system 1000. Further, processes, modules, processor components, and/or other components of system 10 described herein may be executed by one or more processing systems similar to and/or the same as that of computing system 1000.

Computing system 1000 may include one or more processors (e.g., processors 1010a-1010n similar to and/or the same as processor 14 shown in FIG. 2) coupled to system memory 1020, an input/output I/O device interface 1030, and a network interface 1040 via an input/output (I/O) interface 1050. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 1000. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 1020). Computing system 1000 may be a uni-processor system including one processor (e.g., processor 1010a), or a multi-processor system including any number of suitable processors (e.g., 1010a-1010n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 1000 may include a plurality of computing devices (e.g., distributed computer systems) to implement various processing functions.

I/O device interface 1030 may provide an interface for connection of one or more I/O devices 1060 to computer system 1000. I/O devices may include devices that receive input (e.g., from a user) or output information (e.g., to a user). I/O devices 1060 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or other devices. I/O devices 1060 may be connected to computer system 1000 through a wired or wireless connection. I/O devices 1060 may be connected to computer system 1000 from a remote location. I/O devices 1060 located on a remote computer system, for example, may be connected to computer system 1000 via a network and network interface 1040.

Network interface 1040 may include a network adapter that provides for connection of computer system 1000 to a network. Network interface may 1040 may facilitate data exchange between computer system 1000 and other devices connected to the network. Network interface 1040 may support wired or wireless communication. The network may include an electronic communication network, such as the Internet, a local area network (LAN), a wide area network (WAN), a cellular communications network, or other networks.

System memory 1020 may be configured to store program instructions 1070 or data 1080. Program instructions 1070 may be executable by a processor (e.g., one or more of processors 1010a-1010n) to implement one or more embodiments of the present techniques. Instructions 1070 may include modules and/or components (e.g., machine readable instructions 15 shown in FIG. 2) of computer program instructions for implementing one or more techniques described herein with regard to various processing modules and/or components. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 1020 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or other memory. System memory 1020 may include a non-transitory computer readable storage medium that may have program instructions stored thereon that are executable by a computer processor (e.g., one or more of processors 1010a-1010n) to cause the subject matter and the functional operations described herein. A memory (e.g., system memory 1020) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times, e.g., a copy may be created by writing program code to a first-in-first-out buffer in a network interface, where some of the instructions are pushed out of the buffer before other portions of the instructions are written to the buffer, with all of the instructions residing in memory on the buffer, just not all at the same time.

I/O interface 1050 may be configured to coordinate I/O traffic between processors 1010a-1010n, system memory 1020, network interface 1040, I/O devices 1060, and/or other peripheral devices. I/O interface 1050 may perform protocol, timing, or other data transformations to convert data signals from one component (e.g., system memory 1020) into a format suitable for use by another component (e.g., processors 1010a-1010n). I/O interface 1050 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 1000 or multiple computer systems 1000 configured to host different portions or instances of embodiments. Multiple computer systems 1000 may provide for parallel or sequential processing/execution of one or more portions of the techniques described herein.

Those skilled in the art will appreciate that computer system 1000 is merely illustrative and is not intended to limit the scope of the techniques described herein. Computer system 1000 may include any combination of devices or software that may perform or otherwise provide for the performance of the techniques described herein. For example, computer system 1000 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, a television or device connected to a television (e.g., Apple TV™), or a Global Positioning System (GPS), or other devices. Computer system 1000 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 1000 may be transmitted to computer system 1000 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present invention may be practiced with other computer system configurations.

### Example 5—Different Thermal Profile Signatures of Fusion Proteins with S-Tag (Micro-Tag)

### Materials and Methods:

HEK-293 cells were from ATCC (Cat #CRL-1573) and were cultured in DMEM (Millipore SIGMA Cat #D5796) supplemented with 10% Fetal Bovine Serum (FBS) (Millipore SIGMA Cat #F2442), and 1× Penicillin/Streptomycin (Millipore SIGMA Cat #P4333). Trypsin-EDTA solution 1× (0.05% trypsin, 0.02% EDTA) was from Millipore SIGMA (Cat #59417C) and 20×TBS solution was from Teknova (Cat #T1680). Triton-X-100 was from Millipore SIGMA (Cat #X100). The detergent n-Dodecyl beta-D-maltoside (DDM) was from Millipore SIGMA (Cat #D4641). The MTH1 inhibitor (S) Crizotinib was from Millipore SIGMA (Cat #PZ0240). The BCL6 inhibitors BI-3812 and BI-5273 were from Boehringer Ingelheim. DMSO was from Millipore SIGMA (Cat #673439). The 96 well plates were from CELLTREAT Scientific (Cat #229196); white PCR plates were from BIORAD (cat #MLL9651) and the Microseal ‘B’ film for sealing the PCR plates was from BIORAD (Cat #MSB1001); black 96 well plates from COSTAR (Cat #3915). Transfection of the cells was realized using Lipofectamine 2000 transfection reagent (Thermo Fisher Scientific Cat #11668027) according to manufacturer recommended protocol. The optimized fluorogenic substrate (5′-6FAM/ArUAA/3′ TAMRA_(NHS ester) (v3)) was purchased from IDT. The S Protein was cloned with 6×His tag at the carboxy terminus into vector pET-30a(+) for bacterial expression using KPN1 GGTACC and BamH1 GGATCC cloning sites by Synbio Technologies Inc. The S protein His tag fusion protein was expressed and purified from bacteria by Synbio Technologies Inc.

The sequence of the cloned construct encoding the RNAse donor was as follows:

(SEQ ID NO: 22)
GGTACCATGAGCAGCTCCAACTACTGTAACCAGATGATGAAGAGCCGG
AACCTGACCAAAGATCGATGCAAGCCAGTGAACACCTTTGTGCACGAG
TCCCTGGCTGATGTCCAGGCCGTGTGCTCCCAGAAAAATGTTGCCTGC
AAGAATGGGCAGACCAATTGCTACCAGAGCTACTCCACCATGAGCATC
ACCGACTGCCGTGAGACCGGCAGCTCCAAGTACCCCAACTGTGCCTAC
AAGACCACCCAGGCGAATAAACACATCATTGTGGCTTGTGAGGGAAAC
CCGTACGTGCCAGTCCACTTTGATGCTTCAGTGCATCACCATCACCAT
CACTAGGGATCC.

The sequence encoding the 6×His tag is bold and the TAG stop codon is underlined.

The corresponding translated amino acid sequence of the His tagged RNase donor protein (S protein) was:

(SEQ ID NO: 23)
MSSSNYCNQMMKSRNLTKDRCKPVNTFVHESLADVQAVCSQKNVACKN
GQTNCYQSYSTMSITDCRETGSSKYPNCAYKTTQANKHIIVACEGNPY
VPVHFDASVHHHHHH.

Cloning of the S-tag acceptor peptide with the mutT homologue (MTH1) protein (i.e., the target polypeptide) was performed by Synbio Technologies Inc. Cloning of the S-tag acceptor peptide to the carboxy terminal of MTH1 was as follows in vector pcDNA3.1(+): cloning site KPN1 GGTACC and BamH1 GGATCC (bold).

We generated the encoding construct with no linker between MTH1 and the S-tag peptide (underlined) as shown below:

(SEQ ID NO: 24)
GGTACCATGGGCGCCTCCAGGCTCTATACCCTGGTGCTGGTCCTGCAG
CCTCAGCGAGTTCTCCTGGGCATGAAAAAGCGAGGCTTCGGGGCCGGC
CGGTGGAATGGCTTTGGGGGCAAAGTGCAAGAAGGAGAGACCATCGAG
GATGGGGCTAGGAGGGAGCTGCAGGAGGAGAGCGGTCTGACAGTGGAC
GCCCTGCACAAGGTGGGCCAGATCGTGTTTGAGTTCGTGGGCGAGCCT
GAGCTCATGGACGTGCATGTCTTCTGCACAGACAGCATCCAGGGGACC
CCCGTGGAGAGCGACGAAATGCGCCCATGCTGGTTCCAGCTGGATCAG
ATCCCCTTCAAGGACATGTGGCCCGACGACAGCTACTGGTTTCCACTC
CTGCTTCAGAAGAAGAAATTCCACGGGTACTTCAAGTTCCAGGGTCAG
GACACCATCCTGGACTACACACTCCGCGAGGTGGACACGGTCAAGGAA
ACTGCAGCAGCCAAGTTTGAGCGGCAGCACATGGACTCCAGCACTTCC
GCTGCCTAGGCTGCCTAGGGATCC.

The translated amino acid sequence for resulting fusion protein comprising the MTH1 and carboxy terminal S-tag acceptor peptide is shown below. The 20 amino acid sequence of the S-tag acceptor peptide is underlined. It was determined that the S-tag could be shortened to the first 15 amino acids and still act as an S-tag acceptor peptide.

(SEQ ID NO: 25)
MGASRLYTLVLVLQPQRVLLGMKKRGFGAGRWNGFGGKVQEGETIEDG
ARRELQEESGLTVDALHKVGQIVFEFVGEPELMDVHVFCTDSIQGTPV
ESDEMRPCWFQLDQIPFKDMWPDDSYWFPLLLQKKKFHGYFKFQGQDT
ILDYTLREVDTVKETAAAKFERQHMDSSTSAA.

We also generated an encoding construct for a fusion protein comprising a 3 amino acid linker (boxed) between the MTH1 target polypeptide and the S-tag acceptor peptide (underlined) as shown below:

(SEQ ID NO: 26)
GGTACCATGGGCGCCTCCAGGCTCTATACCCTGGTGCTGGTCCTGCAGCC
TCAGCGAGTTCTCCTGGGCATGAAAAAGCGAGGCTTCGGGGCCGGCCGGT
GGAATGGCTTTGGGGGCAAAGTGCAAGAAGGAGAGACCATCGAGGATGGG
GCTAGGAGGGAGCTGCAGGAGGAGAGCGGTCTGACAGTGGACGCCCTGCA
CAAGGTGGGCCAGATCGTGTTTGAGTTCGTGGGCGAGCCTGAGCTCATGG
ACGTGCATGTCTTCTGCACAGACAGCATCCAGGGGACCCCCGTGGAGAGC
GACGAAATGCGCCCATGCTGGTTCCAGCTGGATCAGATCCCCTTCAAGGA
CATGTGGCCCGACGACAGCTACTGGTTTCCACTCCTGCTTCAGAAGAAGA
AATTCCACGGGTACTTCAAGTTCCAGGGTCAGGACACCATCCTGGACTAC

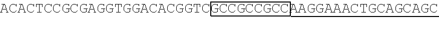

CAAGTTTGAGCGGCAGCACATGGACTCCAGCACTTCCGCTGCCTAGGCTG
CCTAGGGATCC

Synthesis of the BCL6 (B-Cell Lymphoma 6) S-Peptide micro-tag construct was performed at Twist Biosciences and the corresponding nucleotide sequence was:

(SEQ ID NO: 30)
ATGGCCTCGCCGGCTGACAGCTGTATCCAGTTCACCCGCCATGCCAGT
GATGTTCTTCTCAACCTTAATCGTCTCCGGAGTCGAGACATCTTGACT
GATGTTGTCATTGTTGTGAGCCGTGAGCAGTTTAGAGCCCATAAAACG
GTCCTCATGGCCTGCAGTGGCCTGTTCTATAGCATCTTTACAGACCAG
TTGAAATGCAACCTTAGTGTGATCAATCTAGATCCTGAGATCAACCCT
GAGGGATTCTGCATCCTCCTGGACTTCATGTACACATCTCGGCTCAAT
TTGCGGGAGGGCAACATCATGGCTGTGATGGCCACGGCTATGTACCTG
CAGATGGAGCATGTTGTGGACACTTGCCGGAAGTTTATTAAGGCCAGT
GAAGCAGAGATGGTTTCTGCCATCAAGCCTCCTCGTGAAGAGTTCCTC
AACAGCCGGATGCTGATGCCCCAAGACATCATGGCCTATCGGGGTCGT
GAGGTGGTGGAGAACAACCTGCCACTGAGGAGCGCCCCTGGGTGTGAG
AGCAGAGCCTTTGCCCCCAGCCTGTACAGTGGCCTGTCCACACCGCCA
GCCTCTTATTCCATGTACAGCCACCTCCCTGTCAGCAGCCTCCTCTTC
TCCGATGAGGAGTTTCGGGATGTCCGGATGCCTGTGGCCAACCCCTTC
CCCAAGGAGCGGGCACTCCCATGTGATAGTGCCAGGCCAGTCCCTGGT
GAGTACAGCCGGCCGACTTTGGAGGTGTCCCCCAATGTGTGCCACAGC
AATATCTATTCACCCAAGGAAACAATCCCAGAAGAGGCACGAAGTGAT
ATGCACTACAGTGTGGCTGAGGGCCTCAAACCTGCTGCCCCCTCAGCC
CGAAATGCCCCCTACTTCCCTTGTGACAAGGCCAGCAAAGAAGAAGAG
AGACCCTCCTCGGAAGATGAGATTGCCCTGCATTTCGAGCCCCCCAAT
GCACCCCTGAACCGGAAGGGTCTGGTTAGTCCACAGAGCCCCCAGAAA
TCTGACTGCCAGCCCAACTCGCCCACAGAGTCCTGCAGCAGTAAGAAT
GCCTGCATCCTCCAGGCTTCTGGCTCCCCTCCAGCCAAGAGCCCCACT
GACCCCAAAGCCTGCAACTGGAAGAAATACAAGTTCATCGTGCTCAAC
AGCCTCAACCAGAATGCCAAACCAGAGGGGCCTGAGCAGGCTGAGCTG
GGCCGCCTTTCCCCACGAGCCTACACGGCCCCACCTGCCTGCCAGCCA
CCCATGGAGCCTGAGAACCTTGACCTCCAGTCCCCAACCAAGCTGAGT
GCCAGCGGGGAGGACTCCACCATCCCACAAGCCAGCCGGCTCAATAAC
ATCGTTAACAGGTCCATGACGGGCTCTCCCCGCAGCAGCAGCGAGAGC
CACTCACCACTCTACATGCACCCCCCGAAGTGCACGTCCTGCGGCTCT
CAGTCCCCACAGCATGCAGAGATGTGCCTCCACACCGCTGGCCCCACG
TTCCCTGAGGAGATGGGAGAGACCCAGTCTGAGTACTCAGATTCTAGC
TGTGAGAACGGGGCCTTCTTCTGCAATGAGTGTGACTGCCGCTTCTCT
GAGGAGGCCTCACTCAAGAGGCACACGCTGCAGACCCACAGTGACAAA
CCCTACAAGTGTGACCGCTGCCAGGCCTCCTTCCGCTACAAGGGCAAC
CTCGCCAGCCACAAGACCGTCCATACCGGTGAGAAACCCTATCGTTGC
AACATCTGTGGGGCCCAGTTCAACCGGCCAGCCAACCTGAAAACCCAC
ACTCGAATTCACTCTGGAGAGAAGCCCTACAAATGCGAAACCTGCGGA
GCCAGATTTGTACAGGTGGCCCACCTCCGTGCCCATGTGCTTATCCAC
ACTGGTGAGAAGCCCTATCCCTGTGAAATCTGTGGCACCCGTTTCCGG
CACCTTCAGACTCTGAAGAGCCACCTGCGAATCCACACAGGAGAGAAA
CCTTACCATTGTGAGAAGTGTAACCTGCATTTCCGTCACAAAAGCCAG
CTGCGACTTCACTTGCGCCAGAAGCATGGCGCCATCACCAACACCAAG
GTGCAATACCGCGTGTCAGCCACTGACCTGCCTCCGGAGCTCCCCAAA
GCCTGCaaggaaactgcagcagccaagtttgagcggcagcacatggac
tccagcacttccgctgccTGA

The lower case letters identify the S-peptide nucleotide residues at the carboxy terminus. The resulting protein amino acid sequence of the expressed protein is shown below and the S-peptide sequence is underlined:

(SEQ ID NO: 31)
MASPADSCIQFTRHASDVLLNLNRLRSRDILTDVVIVVSREQFRAHKT
VLMACSGLFYSIFTDQLKCNLSVINLDPEINPEGFCILLDFMYTSRLN
LREGNIMAVMATAMYLQMEHVVDTCRKFIKASEAEMVSAIKPPREEFL
NSRMLMPQDIMAYRGREVVENNLPLRSAPGCESRAFAPSLYSGLSTPP
ASYSMYSHLPVSSLLFSDEEFRDVRMPVANPFPKERALPCDSARPVPG
EYSRPTLEVSPNVCHSNIYSPKETIPEEARSDMHYSVAEGLKPAAPSA
RNAPYFPCDKASKEEERPSSEDEIALHFEPPNAPLNRKGLVSPQSPQK
SDCQPNSPTESCSSKNACILQASGSPPAKSPTDPKACNWKKYKFIVLN
SLNQNAKPEGPEQAELGRLSPRAYTAPPACQPPMEPENLDLQSPTKLS
ASGEDSTIPQASRLNNIVNRSMTGSPRSSSESHSPLYMHPPKCTSCGS
QSPQHAEMCLHTAGPTFPEEMGETQSEYSDSSCENGAFFCNECDCRFS
EEASLKRHTLQTHSDKPYKCDRCQASFRYKGNLASHKTVHTGEKPYRC
NICGAQFNRPANLKTHTRIHSGEKPYKCETCGARFVQVAHLRAHVLIH
TGEKPYPCEICGTRFRHLQTLKSHLRIHTGEKPYHCEKCNLHFRHKSQ
LRLHLRQKHGAITNTKVQYRVSATDLPPELPKACKETAAAKFERQHMD
SSTSAA-

Synthesis of the EGFR S-peptide tag construct was performed at Twist Biosciences and the corresponding nucleotide sequence was:

(SEQ ID NO: 32)
atgcgaccctccgggacggccggggcagcgctcctggcgctgctggct
gcgctctgcccggcgagtcgggctctggaggaaaagaaagtttgccaa
ggcacgagtaacaagctcacgcagttgggcacttttgaagatcatttt
ctcagcctccagaggatgttcaataactgtgaggtggtccttgggaat
ttggaaattacctatgtgcagaggaattatgatctttccttcttaaag
accatccaggaggtggctggttatgtcctcattgccctcaacacagtg
gagcgaattcctttggaaaacctgcagatcatcagaggaaatatgtac
tacgaaaattcctatgccttagcagtcttatctaactatgatgcaaat
aaaaccggactgaaggagctgcccatgagaaatttacaggaaatcctg
catggcgccgtgcggttcagcaacaaccctgccctgtgcaacgtggag
agcatccagtggcgggacatagtcagcagtgactttctcagcaacatg
tcgatggacttccagaaccacctgggcagctgccaaaagtgtgatcca
agctgtcccaatgggagctgctggggtgcaggagaggagaactgccag
aaactgaccaaaatcatctgtgcccagcagtgctccgggcgctgccgt
ggcaagtcccccagtgactgctgccacaaccagtgtgctgcaggctgc
acaggcccccgggagagcgactgcctggtctgccgcaaattccgagac
gaagccacgtgcaaggacacctgccccccactcatgctctacaacccc
accacgtaccagatggatgtgaaccccgagggcaaatacagctttggt
gccacctgcgtgaagaagtgtccccgtaattatgtggtgacagatcac
ggctcgtgcgtccgagcctgtggggccgacagctatgagatggaggaa
gacggcgtccgcaagtgtaagaagtgcgaagggccttgccgcaaagtg
tgtaacggaataggtattggtgaatttaaagactcactctccataaat
gctacgaatattaaacacttcaaaaactgcacctccatcagtggcgat
ctccacatcctgccggtggcatttaggggtgactccttcacacatact
cctcctctggatccacaggaactggatattctgaaaaccgtaaaggaa
atcacagggtttttgctgattcaggcttggcctgaaaacaggacggac
ctccatgcctttgagaacctagaaatcatacgcggcaggaccaagcaa
catggtcagttttctcttgcagtcgtcagcctgaacataacatccttg
ggattacgctccctcaaggagataagtgatggagatgtgataatttca
ggaaacaaaaatttgtgctatgcaaatacaataaactggaaaaaactg
tttgggacctccggtcagaaaaccaaaattataagcaacagaggtgaa
aacagctgcaaggccacaggccaggtctgccatgccttgtgctccccc
gagggctgctggggcccggagcccagggactgcgtctcttgccggaat
gtcagccgaggcagggaatgcgtggacaagtgcaaccttctggagggt
gagccaagggagtttgtggagaactctgagtgcatacagtgccaccca
gagtgcctgcctcaggccatgaacatcacctgcacaggacggggacca
gacaactgtatccagtgtgcccactacattgacggcccccactgcgtc
aagacctgcccggcaggagtcatgggagaaaacaacaccctggtctgg
aagtacgcagacgccggccatgtgtgccacctgtgccatccaaactgc
acctacggatgcactgggccaggtcttgaaggctgtccaacgaatggg
cctaagatcccgtccatcgccactgggatggtgggggccctcctcttg
ctgctggtggtggccctggggatcggcctcttcatgcgaaggcgccac
atcgttcggaagcgcacgctgcggaggctgctgcaggagagggagctt
gtggagcctcttacacccagtggagaagctcccaaccaagctctcttg
aggatcttgaaggaaactgaattcaaaaagatcaaagtgctgggctcc
ggtgcgttcggcacggtgtataagggactctggatcccagaaggtgag
aaagttaaaattcccgtcgctatcaaggaattaagagaagcaacatct
ccgaaagccaacaaggaaatcctcgatgaagcctacgtgatggccagc
gtggacaacccccacgtgtgccgcctgctgggcatctgcctcacctcc
accgtgcagctcatcatgcagctcatgcccttcggctgcctcctggac
tatgtccgggaacacaaagacaatattggctcccagtacctgctcaac
tggtgtgtgcagatcgcaaagggcatgaactacttggaggaccgtcgc
ttggtgcaccgcgacctggcagccaggaacgtactggtgaaaacaccg
cagcatgtcaagatcacagattttgggcgggccaaactgctgggtgcg
gaagagaaagaataccatgcagaaggaggcaaagtgcctatcaagtgg
atggcattggaatcaattttacacagaatctatacccaccagagtgat
gtctggagctacggggtgactgtttgggagttgatgacctttggatcc
aagccatatgacggaatccctgccagcgagatctcctccatcctggag
aaaggagaacgcctccctcagccacccatatgtaccatcgatgtctac
atgatcatggtcaagtgctggatgatagacgcagatagtcgcccaaag
ttccgtgagttgatcatcgaattctccaaaatggcccgagacccccag
cgctaccttgtcattcagggggatgaaagaatgcatttgccaagtcct
acagactccaacttctaccgtgccctgatggatgaagaagacatggac
gacgtggtggatgccgacgagtacctcatcccacagcagggcttcttc
agcagcccctccacgtcacggactcccctcctgagctctctgagtgca
accagcaacaattccaccgtggcttgcattgatagaaatgggctgcaa
agctgtcccatcaaggaagacagcttcttgcagcgatacagctcagac
cccacaggcgccttgactgaggacagcatagacgacaccttcctccca
gtgcctgaatacataaaccagtccgttcccaaaaggcccgctggctct
gtgcagaatcctgtctatcacaatcagcctctgaaccccgcgcccagc
agagacccacactaccaggacccccacagcactgcagtgggcaacccc
gagtatctcaacactgtccagcccacctgtgtcaacagcacattcgac
agccctgcccactgggcccagaaaggcagccaccaaattagcctggac
aaccctgactaccagcaggacttctttcccaaggaagccaagccaaat
ggcatctttaagggctccacagctgaaaatgcagaatacctaagggtc
gcgccacaaagcagtgaatttattggagcaaaggaaactgcagcagcc
aagtttgagcggcagcacatggactccagcacttccgctgcctga

The S-peptide nucleotide sequence at the carboxy terminus is underlined. The resulting protein amino acid sequence of the expressed protein is:

(SEQ ID NO: 33)
MRPSGTAGAALLALLAALCPASRALEEKKVCQGTSNKLTQLGTFEDHF
LSLQRMFNNCEVVLGNLEITYVQRNYDLSFLKTIQEVAGYVLIALNTV
ERIPLENLQIIRGNMYYENSYALAVLSNYDANKTGLKELPMRNLQEIL
HGAVRFSNNPALCNVESIQWRDIVSSDFLSNMSMDFQNHLGSCQKCDP
SCPNGSCWGAGEENCQKLTKIICAQQCSGRCRGKSPSDCCHNQCAAGC
TGPRESDCLVCRKFRDEATCKDTCPPLMLYNPTTYQMDVNPEGKYSFG
ATCVKKCPRNYVVTDHGSCVRACGADSYEMEEDGVRKCKKCEGPCRKV
CNGIGIGEFKDSLSINATNIKHFKNCTSISGDLHILPVAFRGDSFTHT
PPLDPQELDILKTVKEITGFLLIQAWPENRTDLHAFENLEIIRGRTKQ
HGQFSLAVVSLNITSLGLRSLKEISDGDVIISGNKNLCYANTINWKKL
FGTSGQKTKIISNRGENSCKATGQVCHALCSPEGCWGPEPRDCVSCRN
VSRGRECVDKCNLLEGEPREFVENSECIQCHPECLPQAMNITCTGRGP
DNCIQCAHYIDGPHCVKTCPAGVMGENNTLVWKYADAGHVCHLCHPNC
TYGCTGPGLEGCPTNGPKIPSIATGMVGALLLLLVVALGIGLFMRRRH
IVRKRTLRRLLQERELVEPLTPSGEAPNQALLRILKETEFKKIKVLGS
GAFGTVYKGLWIPEGEKVKIPVAIKELREATSPKANKEILDEAYVMAS
VDNPHVCRLLGICLTSTVQLIMQLMPFGCLLDYVREHKDNIGSQYLLN
WCVQIAKGMNYLEDRRLVHRDLAARNVLVKTPQHVKITDFGRAKLLGA
EEKEYHAEGGKVPIKWMALESILHRIYTHQSDVWSYGVTVWELMTFGS
KPYDGIPASEISSILEKGERLPQPPICTIDVYMIMVKCWMIDADSRPK
FRELIIEFSKMARDPQRYLVIQGDERMHLPSPTDSNFYRALMDEEDMD
DVVDADEYLIPQQGFFSSPSTSRTPLLSSLSATSNNSTVACIDRNGLQ
SCPIKEDSFLQRYSSDPTGALTEDSIDDTFLPVPEYINQSVPKRPAGS
VQNPVYHNQPLNPAPSRDPHYQDPHSTAVGNPEYLNTVQPTCVNSTFD
SPAHWAQKGSHQISLDNPDYQQDFFPKEAKPNGIFKGSTAENAEYLRV
APQSSEFIGAKETAAAKFERQHMDSSTSAA-

The S-peptide sequence at the carboxy terminus is underlined.

A. Cell Transfection and Lysate Preparation

HEK293 cells were transfected using Lipofectamine 2000 according to manufacturer protocol. Two days after transfection, media was removed and cells lifted by pipetting up and down using 1×TBS. Cells were centrifuged at 400 g for 4 min. The TBS wash was removed and the cells lysed with either 1% Triton X 100 in TBS for MTH1 and BCL6, or 0.5% DDM in TBS for EGFR expressing cells. Cells were lysed at 4° C. on a rotator for 1 hour and cell debris removed by gentle centrifugation for 1 minute at 6000 rpm. Lysates were transferred to a new tube and aliquots that were not used immediately in the assay were frozen at −80° C. for future use.

B. Thermal Profile

The lysates prepared from cells overexpressing the constructs were diluted 1/10 with 1×TBS and 40 ul aliquoted to PCR tubes or plates and heated at a gradient of heat in a thermal cycler for 15 minutes. After heating, the samples were immediately assayed in the enzyme complementation. A mix of 1.25 μg/ml S Protein and 250 nM FRET-labeled nucleic acid substrate in 1×TBS with 0.5% DMSO was combined with the heated sample and fluorescence detected using a microplate reader. The plate was read in kinetic mode (excitation=485 nm, emission=520 nm) with the PolarStar Omega 96-well plate reader. Every plate was read every minute for 20 minutes (21 time points were generated).

For determination of the thermal profile and to determine the T_{agg}, Tₘₐₓ, or Tₘᵢₙ, RAW fluorescence or fluorescence signal generated per minute was plotted with temperature. A sigmoidal plot identifies temperature of aggregation, or maximal signal, or minimal signal.

C. Small Molecule Target Engagement Screening

Small molecule compounds were dissolved in DMSO at 10 mM and serial dilutions in DMSO at 100× concentration were prepared from this stock. Lysates from cells expressing the micro-tagged (S-peptide tagged) protein were diluted with cold 1×TBS at 1/10 and 39.6 ul aliquoted to a PCR plate; 0.4 ul of the 100× small molecule was added, the plate sealed, vortexed and centrifuged for 2 min. The plate was then heated at the appropriate temperature for 15 minutes followed by 1 minute cool down at 25° C. Reaction Buffer (1.25 μg/ml S Protein Binding Partner and 250 nM FRET-labeled substrate in 1×TBS with 0.5% DMSO) was prepared and 120 ul transferred to a black 96 well plate. The heated sample (30 ul) was added and fluorescence detected using a microplate reader. The plate was read in kinetic mode (excitation=485 nm, emission=520 nm) with the PolarStar Omega 96-well plate reader.

For analysis, the RAW fluorescence signal or fluorescence change per minute was plotted with inhibitor dose on a Semi-Log scale using GraphPad Prism. Nonlinear regression analysis was used for curve fitting to identify EC50 of target engagement or apparent K_{D}.

### Results

The application of Micro-tag (S-tag) to cell target engagement relies on the principle of protein thermal melting. Cells or cell lysates expressing Micro-tag construct were put through a thermal gradient to identify a temperature of aggregation. This is a temperature at which a portion of the protein denatures and aggregates thus becoming insoluble. In a thermal shift assay the protein can be rescued from this heat denaturation through binding of a ligand that impacts the conformation of the protein, making it more stable under denaturing conditions such as heat challenge.

A. Thermal Profile of Micro-tagged Targets

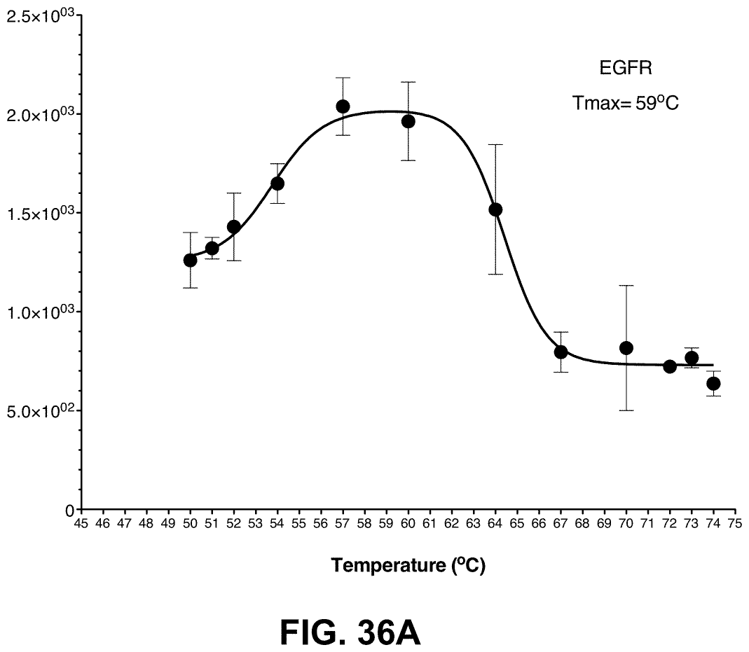

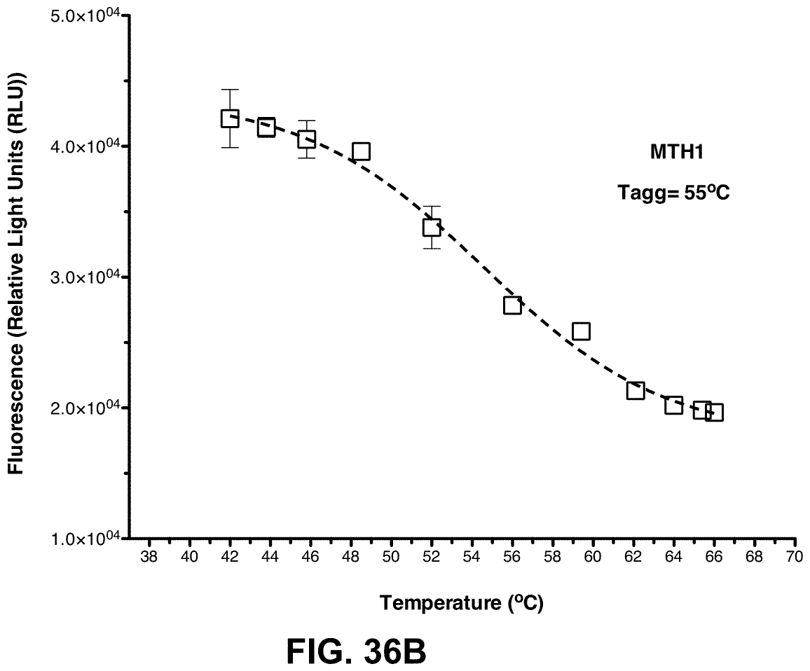

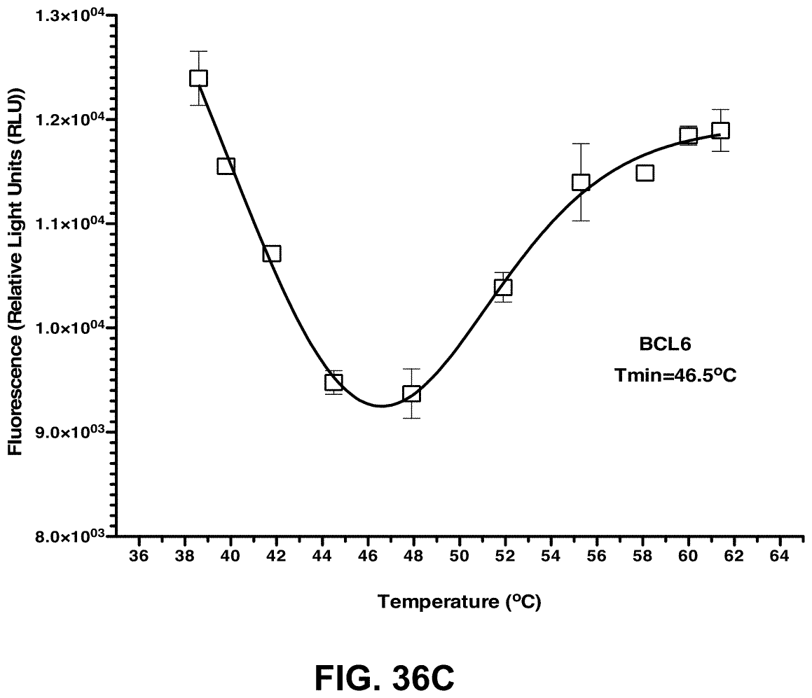

In order to screen for ligands that will bind to a micro-tagged construct, the cells expressing the construct, or non-denaturing lysates prepared from these cells, are put through a thermal gradient to identify a temperature of aggregation for the protein. However, an interesting feature for this Micro-tag system was that thermal challenge to the tagged proteins identified several different thermal signatures: a Temperature of aggregation (T_{agg}), a Temperature of maximal signal (Tₘₐₓ), and a temperature of minimal signal (Tₘᵢₙ) (FIGS. 36A-36C).

Heating cells or lysates from cells overexpressing MTH1 micro-tag (S-peptide tagged) protein at 55° C. (T_{agg}) can be used for screening molecules that will bind and stabilize the MTH1 micro-tagged protein resulting in higher fluorescence signal. The micro-tag (S-peptide) assay system detected fluorescence increase that was directly proportional to the amount of micro-tagged protein in the reaction. The ability to monitor fluorescence signal development in real time combined with the fast enzyme kinetics of the active whole RNAse enzyme (S-peptide complemented with S protein) offered a unique feature not possible with other similar target engagement strategies; monitoring FRET-labeled substrate depletion. As shown in FIG. 34, this system could be used to examine development of fluorescence over time (relative light units (RLU) change per minute) that directly resulted from cleavage of the FRET-labeled substrate. The increase in fluorescence signal within the first 3 minutes of the reaction, the peak fluorescence at about 3 to 4 minutes, and the drop in fluorescence after 4 minutes were all directly proportional to the amount of micro-tagged protein in the assay. This showed the flexibility of the assay in terms of providing different ways to directly quantify the level of the micro-tagged protein.

B. Screening for Molecules Binding to Micro-Tagged Proteins

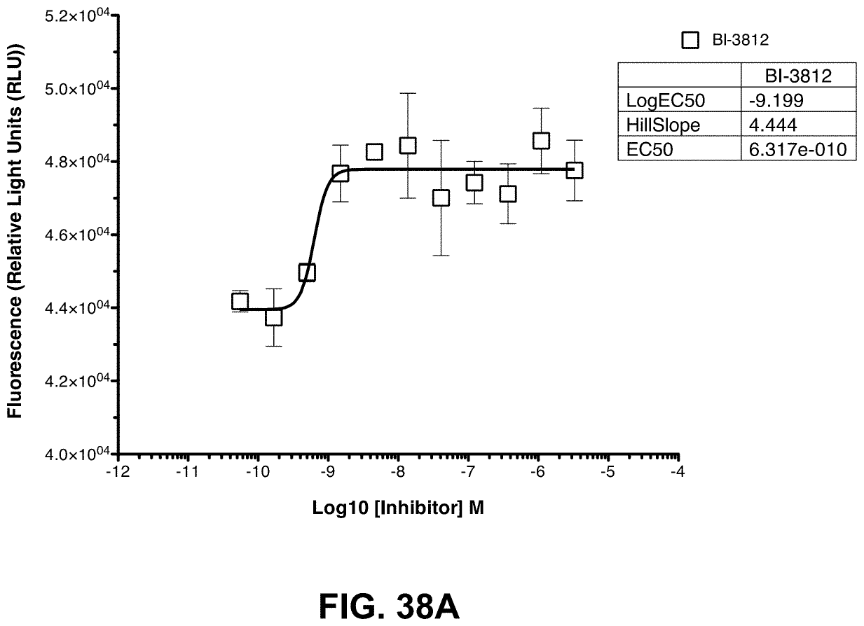

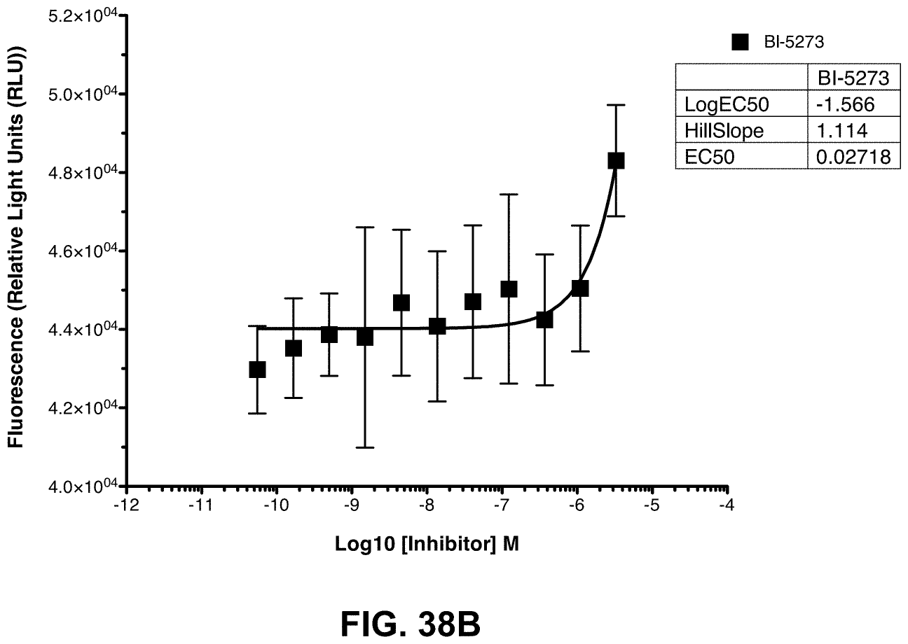

Once the temperature of maximum signal (Tₘₐₓ), temperature of aggregation (T_{agg}), or temperature of minimal signal (Tₘᵢₙ) have been identified for the protein of interest then ligand binding could be assessed at these temperatures. If a ligand bound the micro-tagged protein then a conformational change may occur from the binding event that would stabilize the micro-tagged protein in heat or other denaturation challenge. Here we employed BCL6 Micro-tagged protein together with its well-characterized Boehringer Ingelheim Inhibitor, BI-3812, that has an in vitro IC50 of about 3 nM. A close structural analog of BI-3812 is the inactive compound BI-5273, which has an in vitro IC50 of about 10 uM. Incubation of these inhibitors with cells or lysates from HEK293 cells overexpressing BCL6 Micro-tag resulted in identification of an EC50 of target engagement (FIGS. 38A-38B).

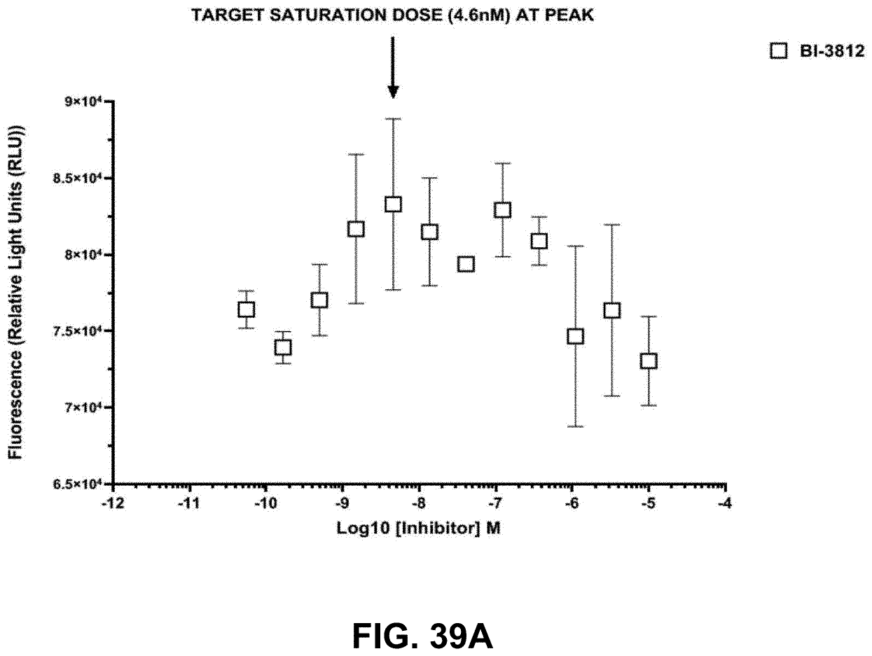

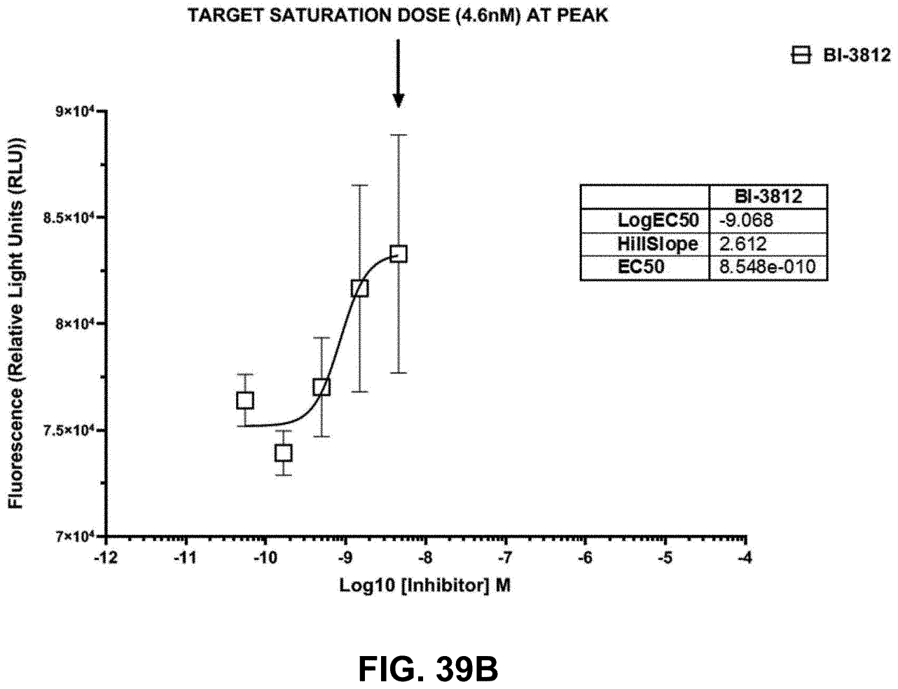

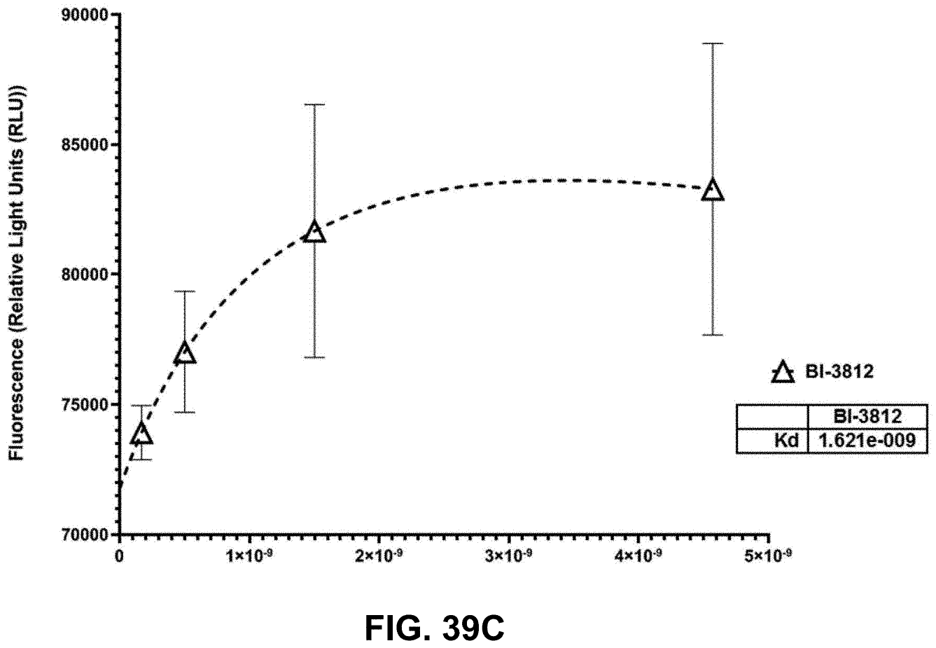

After 4 minutes incubation the fluorescence detected could be plotted with dose of inhibitor tested on a semi-log scale. Using nonlinear regression analysis and fitting a Sigmoidal dose-response curve (with variable slope) using GraphPad Prism identified an EC50 of Target Engagement. The BCL6 specific inhibitor BI-3812 bound to the BCL6 micro-tag protein with an EC50 of target engagement of 0.63 nM (FIGS. 38A-38B). The inactive analog did not bind the target. Allowing the S protein reaction go for longer period of time (10-15 minutes) resulted in deletion of the FRET-labeled substrate as demonstrated by a decreased fluorescence increase per minute (RLU/min) at concentrations of BI-3812 that had stabilized the target. The peak fluorescence signal identified the dose of inhibitor that saturates the micro-tag protein target. At concentrations of the inhibitor above this saturating dose there was rapid cleavage of the FRET-labeled substrate resulting in loss of fluorescence signal over time (Relative Light Unit per minute (RLU/min)) and lower fluorescence detected. At concentrations below this saturating dose the micro-tag construct was denatured in the heat challenge and there was less micro-tag available for enzyme complementation resulting in lower fluorescence signal (FIG. 36A). Removal of data points beyond the target saturation dose allowed for: (1) determination of the EC50 of target engagement, and (2) determination of the Apparent Equilibrium Dissociation Constant (apparent K_{D}) for the drug binding to the protein target (FIGS. 39B and 39C). The EC50 of target engagement determined by this method was very close to the apparent K_{D}, 0.9 nM and 1.6 nM, respectively.

C. Quantitative Binding Kinetic Data in a Physiological Context

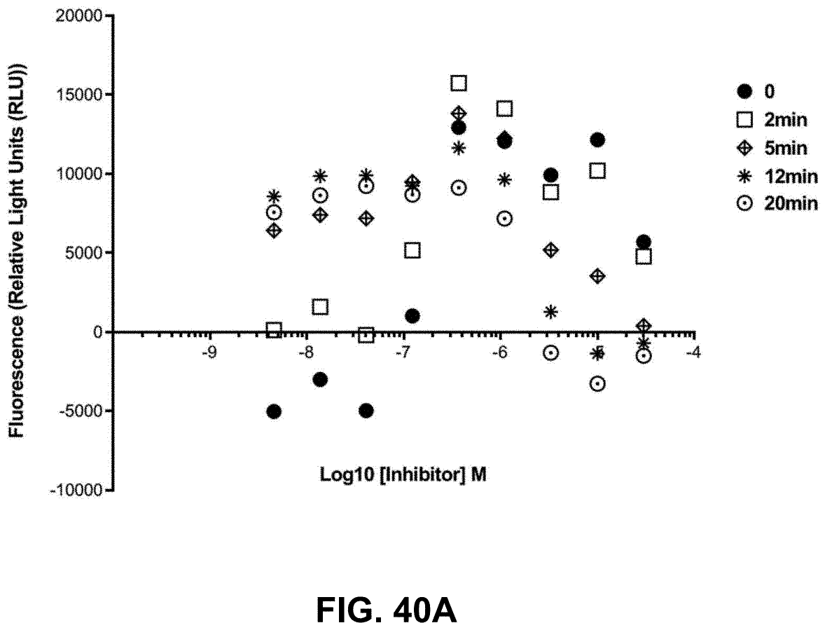

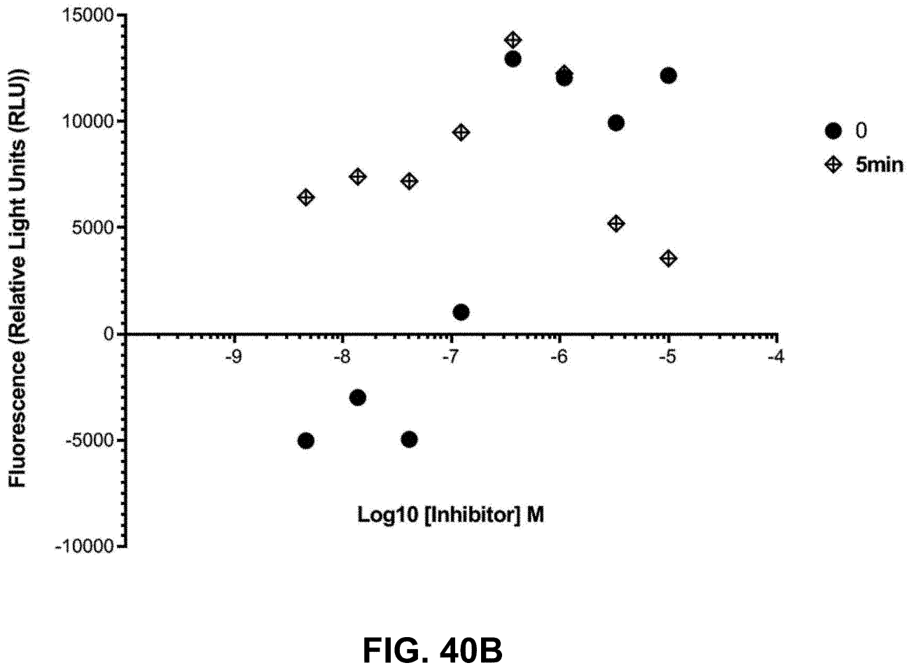

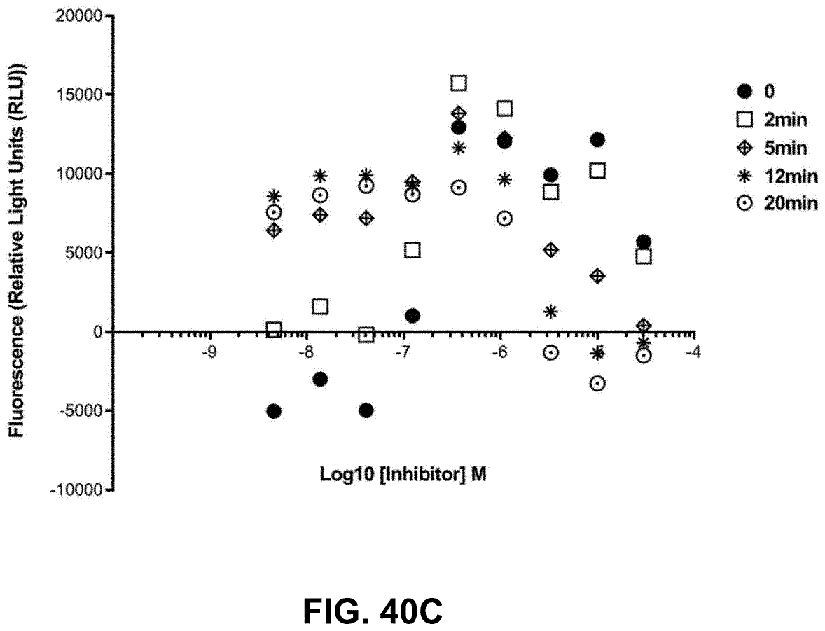

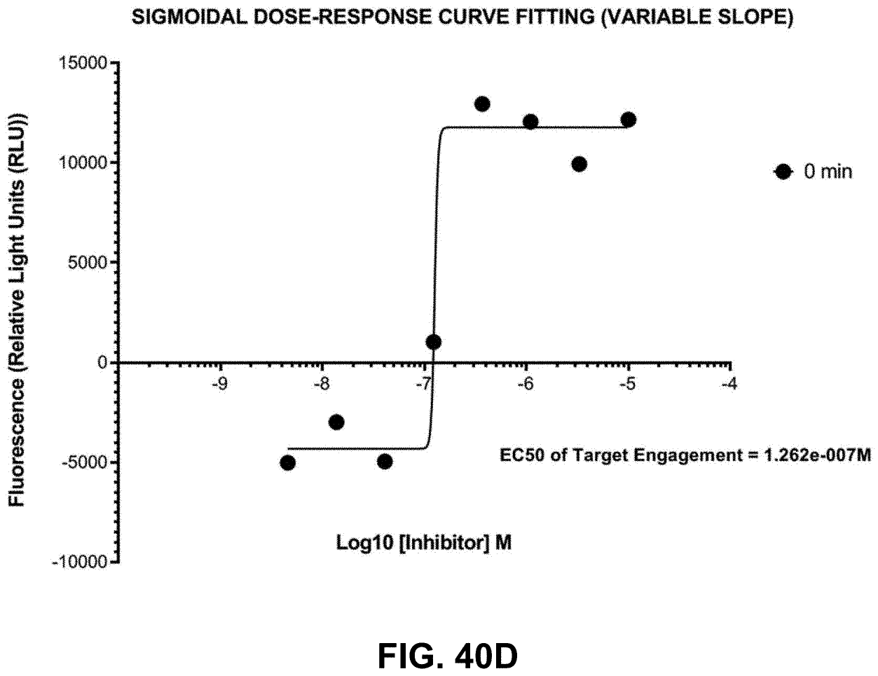

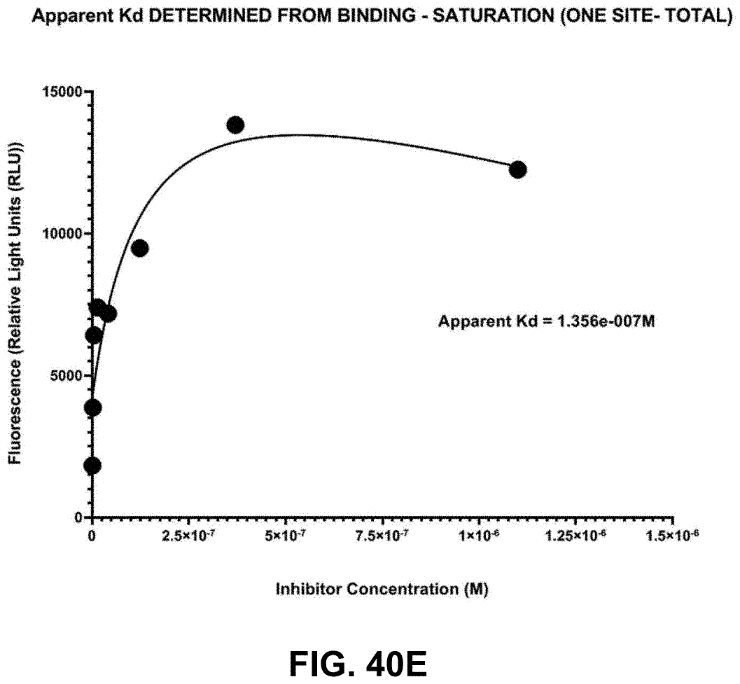

The method may be used to identify the dose of a drug at which the protein target is saturated by the drug to give maximum fluorescence. The target saturation dose could be identified by this method as shown by time course in FIGS. 40A-C. With longer incubation times an inflection point was identified by a drop in fluorescence signal over time, this occurred after 5 minutes (FIG. 40B). The dose at which the signal peaked after longer incubation is the Target Saturation dose (FIG. 40C). It is the inflection point where longer incubations started to result in decreased fluorescence signal over time. The peak fluorescence is referred to as the Emax value (maximum Effect). Knowing the concentration of a drug that saturates a given target is an important parameter for drug discovery as it may be used for defining target occupancy. The EC50 of Target Engagement is determined from a sigmoidal dose-response curve of the Fluorescent Signal versus Drug Concentration on a semi-log scale using the early time points (0 min) before any signal decrease occurred at the higher drug concentrations (FIG. 40D). Nonlinear regression analysis fitting a sigmoidal dose-response curve using GraphPad Prism software identified EC50 of Target Engagement (FIG. 40D and FIG. 39B). The observable fluorescence signal data could also be fit to a Saturation Binding Equation (One-site total) using GraphPad Prism (FIG. 40E and FIG. 39C). This identified an Apparent Equilibrium Dissociation Constant (apparent K_{D}) for the drug binding to the protein target that is similar to the observed EC50 of target engagement (FIG. 40E and FIG. 39C). Fitting observable fluorescence data to a Saturation Binding equation could be performed since the relationship between target binding and fluorescence response was expected to be directly proportional, up to the saturating concentration of drug.

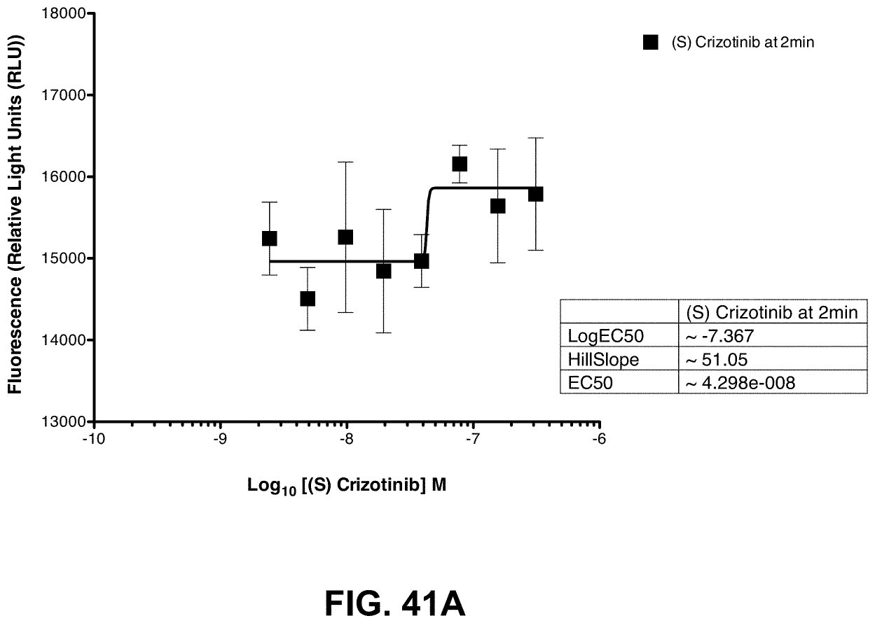

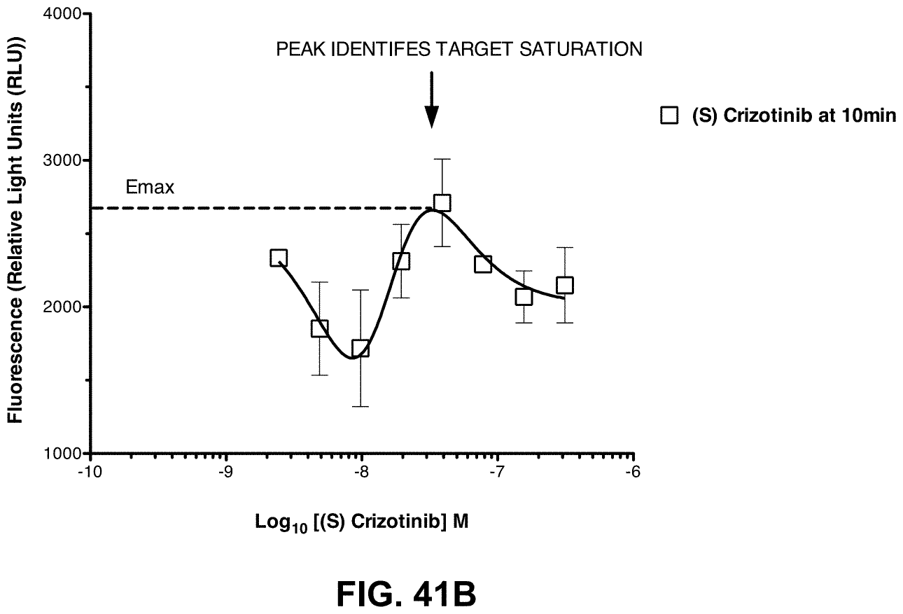

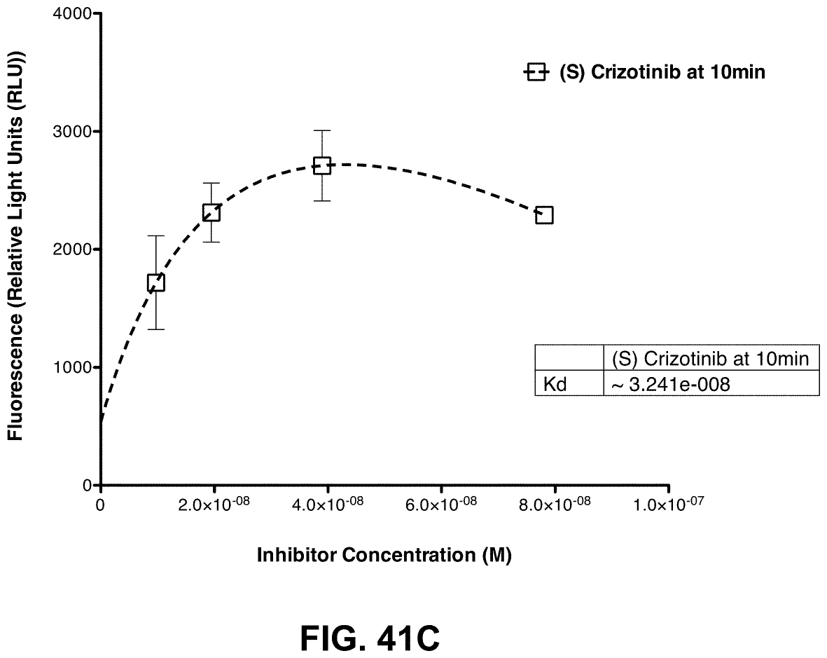

As another example of identifying target saturation dose, Emax and apparent K_{D}, the MTH1 Micro-tagged protein and the inhibitor (S) Crizotinib were tested. Incubating the inhibitor with the protein during a heat challenge followed by enzyme complementation and fluorescence detection resulted in identification of the EC50 of target engagement after 2 minutes (FIG. 41A). With longer incubation times (10 minutes) the fluorescence over time decreased for the higher drug doses. The dose at which the signal peaked after longer incubation is the Target Saturation dose (FIG. 41B). The peak fluorescence was referred to as the Emax value (FIG. 41B). Nonlinear regression analysis fitting a sigmoidal dose-response curve using GraphPad Prism software identifies EC50 of Target Engagement of about 43 nM (FIG. 41A). The observable fluorescence signal data could also be fit to a Saturation Binding Equation (One-site total) using GraphPad Prism (FIG. 41C). This identified an Apparent Equilibrium Dissociation Constant (apparent K_{D}) of 32 nM that was similar to the EC50 of target engagement (43 nM) identified at the 2-minute time point using Sigmoidal dose-response curve fitting.

### Discussion

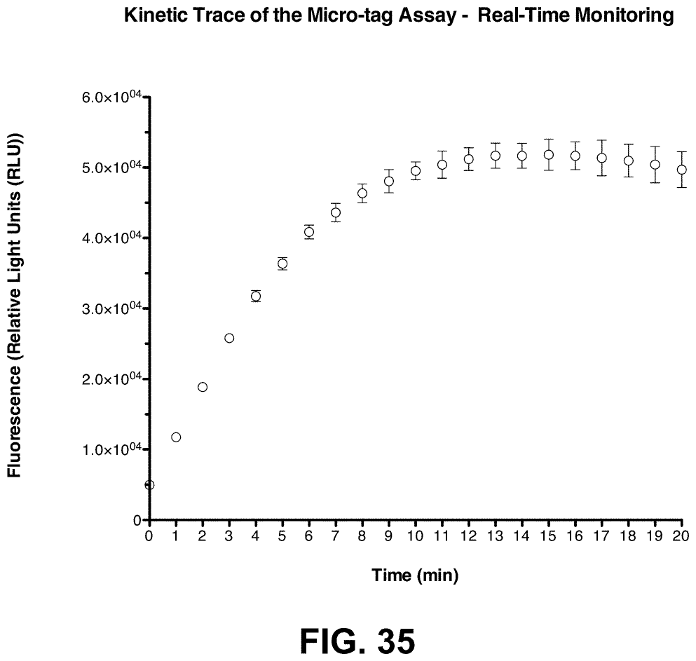

The method employing S-Peptide tag (Micro-tag) had several advantages over other enzyme complementation strategies. Firstly, the short tag (15 to 20 amino acids) was small enough that it did not interfere with the folding, localization, protein-protein interactions, and function of the tagged protein target. Secondly, employing enzyme complementation with the S protein and use of a FRET-labeled nucleic acid substrate for generation of a fluorescent signal had the advantage of being a fast reaction that could be monitored in real time (FIG. 35). This method along with fluorescence detection offered some unique features for this technology. The thermal profiles for some targets identifying a maximum temperature and a minimum temperature of protein melting had an advantage (FIGS. 36A-36C). Proteins with Tₘₐₓ and Tₘᵢₙ could be screened for ligand binding at those temperatures that were generally significantly lower than aggregation temperatures.

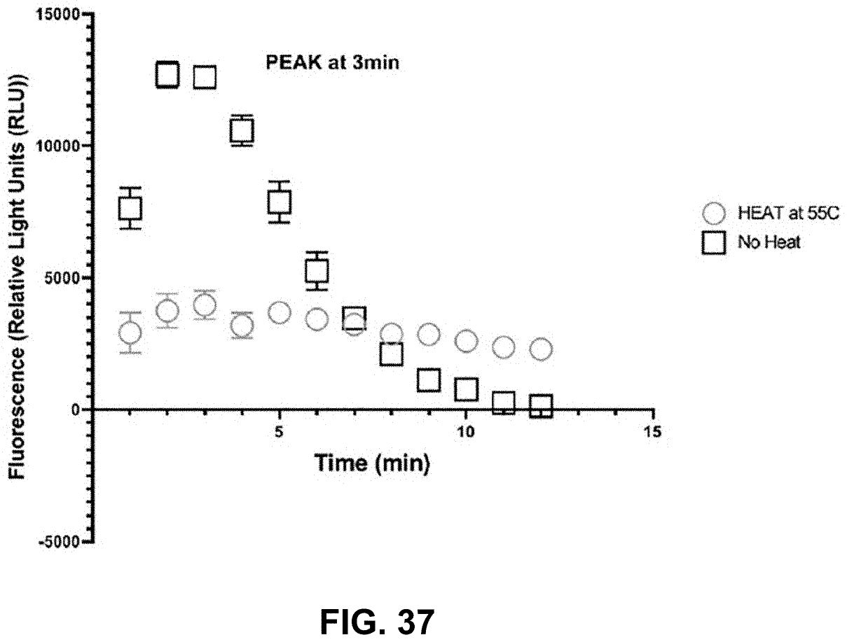

A feature of this technology was the speed of the reaction (FIG. 37). The rapid enzymatic cleavage of FRET-labeled substrate allowed for the determination of an EC50 of target engagement within the first 5 minutes of enzyme complementation. This could then be followed over time to identify the dose at which a small molecule saturated a protein target. The peak fluorescence value (Emax) after depletion of FRET-labeled substrates was used to identify the saturating dose. The speed of this RNase cleavage of the FRET-labeled nucleic acid substrate resulted in depletion of the FRET-labeled substrate within 10 to 20 minutes. This was detected in real time as a decrease in the fluorescence signal over time. This peak fluorescence (target saturation dose) resulted when the increase in signal from stabilization of the target protein was balanced by the decrease in signal from excess stabilized protein target (rapid FRET depletion). The dose at which a small molecule saturates its target was useful for quantifying target occupancy, a feature highly relevant to drug discovery.

The ability to perform quantitative kinetic analysis of small molecule binding to its protein target in a physiological setting was provided as the fluorescent readout, was directly a result of ligand-bound stabilized micro-tagged protein. The apparent equilibrium dissociation constant (K_{D}) could be determined since the target saturation dose was identified in the reaction. Eliminating the doses beyond the saturating dose allowed for Saturation Binding curves that could provide the apparent K_{D} for the small molecule binding to the target. The EC50 of target engagement that was determined from the early time points before depletion of the FRET-labeled substrate correlated with the apparent K_{D} measure determined from Saturation Binding Curve.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. The terms and expressions which have been employed are used as terms of description and not of limitation, and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition. The phrase, “substantially simultaneously” means at the time, or occurring within a time frame of seconds (e.g. within a window of 0 to 10 seconds).

Thus, it should be understood that although the present technology has been specifically disclosed by representative embodiments and optional features, modification and variation of the concepts herein disclosed can be resorted to by those skilled in the art, and such modifications and variations are considered within the scope of this technology.

INFORMAL SEQUENCE LISTING:

KETAAAKFERQHMDSSTSAA (SEQ ID NO: 1)
KETNWAWFWDQHMDSSTSA (SEQ ID NO: 2)
KETGWALFVQQHMDSSTSA (SEQ ID NO: 3)
KETVMANFQMQHMDSSTSA (SEQ ID NO: 4)
KETGDAVFARQHMDSSTSA (SEQ ID NO: 5)
KETGWAAFVKQHMDSSTSA (SEQ ID NO: 6)
KETGWATFVEQHMDSSTSA (SEQ ID NO: 7)
KETKLAFFLKQHMDSSTSA (SEQ ID NO: 8)
KETWWAWFFGQHMDSSTSA (SEQ ID NO: 9)
KETTWAEFTWQHMDSSTSA (SEQ ID NO: 10)
KETPWASFNKQHMDSSTSA (SEQ ID NO: 11)
KETAMAMFVTQHMDSSTSA (SEQ ID NO: 12)
KETLWAWFMWQHMDSSTSA (SEQ ID NO: 13)
KETAAAKFERQHMDS (SEQ ID NO: 14)
KETAAAKFERQHMNS (SEQ ID NO: 15)
NRAWSEFLWQHLAPV (SEQ ID NO: 16)
NRGWSEFLWQHHAPV (SEQ ID NO: 17)
NRAWSVFQWQHIAPA (SEQ ID NO: 18)
MSSSNYCNQMMKSRNLTKDRCKPVNTFVHESLADVQAVCSQKNVACKNGQTNCYQSYSTMSITDCRETGSSKYPNC
AYKTTQANKHIIVACEGNPYVPVHFDASV (SEQ ID NO: 19)
MSSSNYCNQMMKSRNLTKDRCKPVNTFVHESLADVQAVCSQKNVACKNGQTNCYQSYSTMSITDCRETGSSKYPNC
AYKTTQANKHIIVACEGNPYVPVHFD (SEQ ID NO: 20)
KETAAAKFERQHMDSSTSAASSSNYCNQMMKSRNLTKDRCKPVNTFVHESLADVQAVCSQKNVACKNGQTNCYQSY
STMSITDCRETGSSKYPNCAYKTTQANKHIIVACEGNPYVPVHFDASV (SEQ ID NO: 21)
GGTACCATGAGCAGCTCCAACTACTGTAACCAGATGATGAAGAGCCGGAACCTGACCAAAGATCGATGCAAGCCAG
TGAACACCTTTGTGCACGAGTCCCTGGCTGATGTCCAGGCCGTGTGCTCCCAGAAAAATGTTGCCTGCAAGAATGG
GCAGACCAATTGCTACCAGAGCTACTCCACCATGAGCATCACCGACTGCCGTGAGACCGGCAGCTCCAAGTACCCC
AACTGTGCCTACAAGACCACCCAGGCGAATAAACACATCATTGTGGCTTGTGAGGGAAACCCGTACGTGCCAGTCC
ACTTTGATGCTTCAGTGCATCACCATCACCATCACTAGGGATCC (SEQ ID NO: 22)
MSSSNYCNQMMKSRNLTKDRCKPVNTFVHESLADVQAVCSQKNVACKNGQTNCYQSYSTMSITDCRETGSSKYPNC
AYKTTQANKHIIVACEGNPYVPVHFDASVHHHHHH (SEQ ID NO: 23)
GGTACCATGGGCGCCTCCAGGCTCTATACCCTGGTGCTGGTCCTGCAGCCTCAGCGAGTTCTCCTGGGCATGAAAA
AGCGAGGCTTCGGGGCCGGCCGGTGGAATGGCTTTGGGGGCAAAGTGCAAGAAGGAGAGACCATCGAGGATGGGGC
TAGGAGGGAGCTGCAGGAGGAGAGCGGTCTGACAGTGGACGCCCTGCACAAGGTGGGCCAGATCGTGTTTGAGTTC
GTGGGCGAGCCTGAGCTCATGGACGTGCATGTCTTCTGCACAGACAGCATCCAGGGGACCCCCGTGGAGAGCGACG
AAATGCGCCCATGCTGGTTCCAGCTGGATCAGATCCCCTTCAAGGACATGTGGCCCGACGACAGCTACTGGTTTCC
ACTCCTGCTTCAGAAGAAGAAATTCCACGGGTACTTCAAGTTCCAGGGTCAGGACACCATCCTGGACTACACACTC
CGCGAGGTGGACACGGTCAAGGAAACTGCAGCAGCCAAGTTTGAGCGGCAGCACATGGACTCCAGCACTTCCGCTG
CCTAGGCTGCCTAGGGATCC (SEQ ID NO: 24)
MGASRLYTLVLVLQPQRVLLGMKKRGFGAGRWNGFGGKVQEGETIEDGARRELQEESGLTVDALHKVGQIVFEFVG
EPELMDVHVFCTDSIQGTPVESDEMRPCWFQLDQIPFKDMWPDDSYWFPLLLQKKKFHGYFKFQGQDTILDYTLRE
VDTVKETAAAKFERQHMDSSTSAA (SEQ ID NO: 25)
GGTACCATGGGCGCCTCCAGGCTCTATACCCTGGTGCTGGTCCTGCAGCCTCAGCGAGTTCTCCTGGGCATGAAAA
AGCGAGGCTTCGGGGCCGGCCGGTGGAATGGCTTTGGGGGCAAAGTGCAAGAAGGAGAGACCATCGAGGATGGGGC
TAGGAGGGAGCTGCAGGAGGAGAGCGGTCTGACAGTGGACGCCCTGCACAAGGTGGGCCAGATCGTGTTTGAGTTC
GTGGGCGAGCCTGAGCTCATGGACGTGCATGTCTTCTGCACAGACAGCATCCAGGGGACCCCCGTGGAGAGCGACG
AAATGCGCCCATGCTGGTTCCAGCTGGATCAGATCCCCTTCAAGGACATGTGGCCCGACGACAGCTACTGGTTTCC
ACTCCTGCTTCAGAAGAAGAAATTCCACGGGTACTTCAAGTTCCAGGGTCAGGACACCATCCTGGACTACACACTC
CGCGAGGTGGACACGGTCGCCGCCGCCAAGGAAACTGCAGCAGCCAAGTTTGAGCGGCAGCACATGGACTCCAGCA
CTTCCGCTGCCTAGGCTGCCTAGGGATCC (SEQ ID NO: 26)
MGASRLYTLVLVLQPQRVLLGMKKRGFGAGRWNGFGGKVQEGETIEDGARRELQEESGLTVDALHKVGQIVFEFVG
EPELMDVHVFCTDSIQGTPVESDEMRPCWFQLDQIPFKDMWPDDSYWFPLLLQKKKFHGYFKFQGQDTILDYTLRE
VDTVAAAKETAAAKFERQHMDSSTSAA (SEQ ID NO: 27)
GGTACCATGGGCGCCTCCAGGCTCTATACCCTGGTGCTGGTCCTGCAGCCTCAGCGAGTTCTCCTGGGCATGAAAA
AGCGAGGCTTCGGGGCCGGCCGGTGGAATGGCTTTGGGGGCAAAGTGCAAGAAGGAGAGACCATCGAGGATGGGGC
TAGGAGGGAGCTGCAGGAGGAGAGCGGTCTGACAGTGGACGCCCTGCACAAGGTGGGCCAGATCGTGTTTGAGTTC
GTGGGCGAGCCTGAGCTCATGGACGTGCATGTCTTCTGCACAGACAGCATCCAGGGGACCCCCGTGGAGAGCGACG
AAATGCGCCCATGCTGGTTCCAGCTGGATCAGATCCCCTTCAAGGACATGTGGCCCGACGACAGCTACTGGTTTCC
ACTCCTGCTTCAGAAGAAGAAATTCCACGGGTACTTCAAGTTCCAGGGTCAGGACACCATCCTGGACTACACACTC
CGCGAGGTGGACACGGTCGCCGCCGCCGCCGCCGCCGCCGCCGCCGCCAAGGAAACTGCAGCAGCCAAGTTTGAGC
GGCAGCACATGGACTCCAGCACTTCCGCTGCCTAGGCTGCCTAGGGATCC (SEQ ID NO: 28)
MGASRLYTLVLVLQPQRVLLGMKKRGFGAGRWNGFGGKVQEGETIEDGARRELQEESGLTVDALHKVGQIVFEFVG
EPELMDVHVFCTDSIQGTPVESDEMRPCWFQLDQIPFKDMWPDDSYWFPLLLQKKKFHGYFKFQGQDTILDYTLRE
VDTVAAAAAAAAAAKETAAAKFERQHMDSSTSAA (SEQ ID NO: 29)
ATGGCCTCGCCGGCTGACAGCTGTATCCAGTTCACCCGCCATGCCAGTGATGTTCTTCTCAACCTTAATCGTCTCC
GGAGTCGAGACATCTTGACTGATGTTGTCATTGTTGTGAGCCGTGAGCAGTTTAGAGCCCATAAAACGGTCCTCAT
GGCCTGCAGTGGCCTGTTCTATAGCATCTTTACAGACCAGTTGAAATGCAACCTTAGTGTGATCAATCTAGATCCT
GAGATCAACCCTGAGGGATTCTGCATCCTCCTGGACTTCATGTACACATCTCGGCTCAATTTGCGGGAGGGCAACA
TCATGGCTGTGATGGCCACGGCTATGTACCTGCAGATGGAGCATGTTGTGGACACTTGCCGGAAGTTTATTAAGGC
CAGTGAAGCAGAGATGGTTTCTGCCATCAAGCCTCCTCGTGAAGAGTTCCTCAACAGCCGGATGCTGATGCCCCAA
GACATCATGGCCTATCGGGGTCGTGAGGTGGTGGAGAACAACCTGCCACTGAGGAGCGCCCCTGGGTGTGAGAGCA
GAGCCTTTGCCCCCAGCCTGTACAGTGGCCTGTCCACACCGCCAGCCTCTTATTCCATGTACAGCCACCTCCCTGT
CAGCAGCCTCCTCTTCTCCGATGAGGAGTTTCGGGATGTCCGGATGCCTGTGGCCAACCCCTTCCCCAAGGAGCGG
GCACTCCCATGTGATAGTGCCAGGCCAGTCCCTGGTGAGTACAGCCGGCCGACTTTGGAGGTGTCCCCCAATGTGT
GCCACAGCAATATCTATTCACCCAAGGAAACAATCCCAGAAGAGGCACGAAGTGATATGCACTACAGTGTGGCTGA
GGGCCTCAAACCTGCTGCCCCCTCAGCCCGAAATGCCCCCTACTTCCCTTGTGACAAGGCCAGCAAAGAAGAAGAG
AGACCCTCCTCGGAAGATGAGATTGCCCTGCATTTCGAGCCCCCCAATGCACCCCTGAACCGGAAGGGTCTGGTTA
GTCCACAGAGCCCCCAGAAATCTGACTGCCAGCCCAACTCGCCCACAGAGTCCTGCAGCAGTAAGAATGCCTGCAT
CCTCCAGGCTTCTGGCTCCCCTCCAGCCAAGAGCCCCACTGACCCCAAAGCCTGCAACTGGAAGAAATACAAGTTC
ATCGTGCTCAACAGCCTCAACCAGAATGCCAAACCAGAGGGGCCTGAGCAGGCTGAGCTGGGCCGCCTTTCCCCAC
GAGCCTACACGGCCCCACCTGCCTGCCAGCCACCCATGGAGCCTGAGAACCTTGACCTCCAGTCCCCAACCAAGCT
GAGTGCCAGCGGGGAGGACTCCACCATCCCACAAGCCAGCCGGCTCAATAACATCGTTAACAGGTCCATGACGGGC
TCTCCCCGCAGCAGCAGCGAGAGCCACTCACCACTCTACATGCACCCCCCGAAGTGCACGTCCTGCGGCTCTCAGT
CCCCACAGCATGCAGAGATGTGCCTCCACACCGCTGGCCCCACGTTCCCTGAGGAGATGGGAGAGACCCAGTCTGA
GTACTCAGATTCTAGCTGTGAGAACGGGGCCTTCTTCTGCAATGAGTGTGACTGCCGCTTCTCTGAGGAGGCCTCA
CTCAAGAGGCACACGCTGCAGACCCACAGTGACAAACCCTACAAGTGTGACCGCTGCCAGGCCTCCTTCCGCTACA
AGGGCAACCTCGCCAGCCACAAGACCGTCCATACCGGTGAGAAACCCTATCGTTGCAACATCTGTGGGGCCCAGTT
CAACCGGCCAGCCAACCTGAAAACCCACACTCGAATTCACTCTGGAGAGAAGCCCTACAAATGCGAAACCTGCGGA
GCCAGATTTGTACAGGTGGCCCACCTCCGTGCCCATGTGCTTATCCACACTGGTGAGAAGCCCTATCCCTGTGAAA
TCTGTGGCACCCGTTTCCGGCACCTTCAGACTCTGAAGAGCCACCTGCGAATCCACACAGGAGAGAAACCTTACCA
TTGTGAGAAGTGTAACCTGCATTTCCGTCACAAAAGCCAGCTGCGACTTCACTTGCGCCAGAAGCATGGCGCCATC
ACCAACACCAAGGTGCAATACCGCGTGTCAGCCACTGACCTGCCTCCGGAGCTCCCCAAAGCCTGCaaggaaactg
cagcagccaagtttgagcggcagcacatggactccagcacttccgctgccTGA (SEQ ID NO: 30)
MASPADSCIQFTRHASDVLLNLNRLRSRDILTDVVIVVSREQFRAHKTVLMACSGLFYSIFTDQLKCNLSVINLDP
EINPEGFCILLDFMYTSRLNLREGNIMAVMATAMYLQMEHVVDTCRKFIKASEAEMVSAIKPPREEFLNSRMLMPQ
DIMAYRGREVVENNLPLRSAPGCESRAFAPSLYSGLSTPPASYSMYSHLPVSSLLFSDEEFRDVRMPVANPFPKER
ALPCDSARPVPGEYSRPTLEVSPNVCHSNIYSPKETIPEEARSDMHYSVAEGLKPAAPSARNAPYFPCDKASKEEE
RPSSEDEIALHFEPPNAPLNRKGLVSPQSPQKSDCQPNSPTESCSSKNACILQASGSPPAKSPTDPKACNWKKYKF
IVLNSLNQNAKPEGPEQAELGRLSPRAYTAPPACQPPMEPENLDLQSPTKLSASGEDSTIPQASRLNNIVNRSMTG
SPRSSSESHSPLYMHPPKCTSCGSQSPQHAEMCLHTAGPTFPEEMGETQSEYSDSSCENGAFFCNECDCRFSEEAS
LKRHTLQTHSDKPYKCDRCQASFRYKGNLASHKTVHTGEKPYRCNICGAQFNRPANLKTHTRIHSGEKPYKCETCG
ARFVQVAHLRAHVLIHTGEKPYPCEICGTRFRHLQTLKSHLRIHTGEKPYHCEKCNLHFRHKSQLRLHLRQKHGAI
TNTKVQYRVSATDLPPELPKACKETAAAKFERQHMDSSTSAA-(SEQ ID NO: 31)
atgcgaccctccgggacggccggggcagcgctcctggcgctgctggctgcgctctgcccggcgagtcgggctctgg
aggaaaagaaagtttgccaaggcacgagtaacaagctcacgcagttgggcacttttgaagatcattttctcagcct
ccagaggatgttcaataactgtgaggtggtccttgggaatttggaaattacctatgtgcagaggaattatgatctt
tccttcttaaagaccatccaggaggtggctggttatgtcctcattgccctcaacacagtggagcgaattcctttgg
aaaacctgcagatcatcagaggaaatatgtactacgaaaattcctatgccttagcagtcttatctaactatgatgc
aaataaaaccggactgaaggagctgcccatgagaaatttacaggaaatcctgcatggcgccgtgcggttcagcaac
aaccctgccctgtgcaacgtggagagcatccagtggcgggacatagtcagcagtgactttctcagcaacatgtcga
tggacttccagaaccacctgggcagctgccaaaagtgtgatccaagctgtcccaatgggagctgctggggtgcagg
agaggagaactgccagaaactgaccaaaatcatctgtgcccagcagtgctccgggcgctgccgtggcaagtccccc
agtgactgctgccacaaccagtgtgctgcaggctgcacaggcccccgggagagcgactgcctggtctgccgcaaat
tccgagacgaagccacgtgcaaggacacctgccccccactcatgctctacaaccccaccacgtaccagatggatgt
gaaccccgagggcaaatacagctttggtgccacctgcgtgaagaagtgtccccgtaattatgtggtgacagatcac
ggctcgtgcgtccgagcctgtggggccgacagctatgagatggaggaagacggcgtccgcaagtgtaagaagtgcg
aagggccttgccgcaaagtgtgtaacggaataggtattggtgaatttaaagactcactctccataaatgctacgaa
tattaaacacttcaaaaactgcacctccatcagtggcgatctccacatcctgccggtggcatttaggggtgactcc
ttcacacatactcctcctctggatccacaggaactggatattctgaaaaccgtaaaggaaatcacagggtttttgc
tgattcaggcttggcctgaaaacaggacggacctccatgcctttgagaacctagaaatcatacgcggcaggaccaa
gcaacatggtcagttttctcttgcagtcgtcagcctgaacataacatccttgggattacgctccctcaaggagata
agtgatggagatgtgataatttcaggaaacaaaaatttgtgctatgcaaatacaataaactggaaaaaactgtttg
ggacctccggtcagaaaaccaaaattataagcaacagaggtgaaaacagctgcaaggccacaggccaggtctgcca
tgccttgtgctcccccgagggctgctggggcccggagcccagggactgcgtctcttgccggaatgtcagccgaggc
agggaatgcgtggacaagtgcaaccttctggagggtgagccaagggagtttgtggagaactctgagtgcatacagt
gccacccagagtgcctgcctcaggccatgaacatcacctgcacaggacggggaccagacaactgtatccagtgtgc
ccactacattgacggcccccactgcgtcaagacctgcccggcaggagtcatgggagaaaacaacaccctggtctgg
aagtacgcagacgccggccatgtgtgccacctgtgccatccaaactgcacctacggatgcactgggccaggtcttg
aaggctgtccaacgaatgggcctaagatcccgtccatcgccactgggatggtgggggccctcctcttgctgctggt
ggtggccctggggatcggcctcttcatgcgaaggcgccacatcgttcggaagcgcacgctgcggaggctgctgcag
gagagggagcttgtggagcctcttacacccagtggagaagctcccaaccaagctctcttgaggatcttgaaggaaa
ctgaattcaaaaagatcaaagtgctgggctccggtgcgttcggcacggtgtataagggactctggatcccagaagg
tgagaaagttaaaattcccgtcgctatcaaggaattaagagaagcaacatctccgaaagccaacaaggaaatcctc
gatgaagcctacgtgatggccagcgtggacaacccccacgtgtgccgcctgctgggcatctgcctcacctccaccg
tgcagctcatcatgcagctcatgcccttcggctgcctcctggactatgtccgggaacacaaagacaatattggctc
ccagtacctgctcaactggtgtgtgcagatcgcaaagggcatgaactacttggaggaccgtcgcttggtgcaccgc
gacctggcagccaggaacgtactggtgaaaacaccgcagcatgtcaagatcacagattttgggcgggccaaactgc
tgggtgcggaagagaaagaataccatgcagaaggaggcaaagtgcctatcaagtggatggcattggaatcaatttt
acacagaatctatacccaccagagtgatgtctggagctacggggtgactgtttgggagttgatgacctttggatcc
aagccatatgacggaatccctgccagcgagatctcctccatcctggagaaaggagaacgcctccctcagccaccca
tatgtaccatcgatgtctacatgatcatggtcaagtgctggatgatagacgcagatagtcgcccaaagttccgtga
gttgatcatcgaattctccaaaatggcccgagacccccagcgctaccttgtcattcagggggatgaaagaatgcat
ttgccaagtcctacagactccaacttctaccgtgccctgatggatgaagaagacatggacgacgtggtggatgccg
acgagtacctcatcccacagcagggcttcttcagcagcccctccacgtcacggactcccctcctgagctctctgag
tgcaaccagcaacaattccaccgtggcttgcattgatagaaatgggctgcaaagctgtcccatcaaggaagacagc
ttcttgcagcgatacagctcagaccccacaggcgccttgactgaggacagcatagacgacaccttcctcccagtgc
ctgaatacataaaccagtccgttcccaaaaggcccgctggctctgtgcagaatcctgtctatcacaatcagcctct
gaaccccgcgcccagcagagacccacactaccaggacccccacagcactgcagtgggcaaccccgagtatctcaac
actgtccagcccacctgtgtcaacagcacattcgacagccctgcccactgggcccagaaaggcagccaccaaatta
gcctggacaaccctgactaccagcaggacttctttcccaaggaagccaagccaaatggcatctttaagggctccac
agctgaaaatgcagaatacctaagggtcgcgccacaaagcagtgaatttattggagcaaaggaaactgcagcagcc
aagtttgagcggcagcacatggactccagcacttccgctgcctga (SEQ ID NO: 32)
MRPSGTAGAALLALLAALCPASRALEEKKVCQGTSNKLTQLGTFEDHFLSLQRMFNNCEVVLGNLEITYVQRNYDL
SFLKTIQEVAGYVLIALNTVERIPLENLQIIRGNMYYENSYALAVLSNYDANKTGLKELPMRNLQEILHGAVRFSN
NPALCNVESIQWRDIVSSDFLSNMSMDFQNHLGSCQKCDPSCPNGSCWGAGEENCQKLTKIICAQQCSGRCRGKSP
SDCCHNQCAAGCTGPRESDCLVCRKFRDEATCKDTCPPLMLYNPTTYQMDVNPEGKYSFGATCVKKCPRNYVVTDH
GSCVRACGADSYEMEEDGVRKCKKCEGPCRKVCNGIGIGEFKDSLSINATNIKHFKNCTSISGDLHILPVAFRGDS
FTHTPPLDPQELDILKTVKEITGFLLIQAWPENRTDLHAFENLEIIRGRTKQHGQFSLAVVSLNITSLGLRSLKEI
SDGDVIISGNKNLCYANTINWKKLFGTSGQKTKIISNRGENSCKATGQVCHALCSPEGCWGPEPRDCVSCRNVSRG
RECVDKCNLLEGEPREFVENSECIQCHPECLPQAMNITCTGRGPDNCIQCAHYIDGPHCVKTCPAGVMGENNTLVW
KYADAGHVCHLCHPNCTYGCTGPGLEGCPTNGPKIPSIATGMVGALLLLLVVALGIGLFMRRRHIVRKRTLRRLLQ
ERELVEPLTPSGEAPNQALLRILKETEFKKIKVLGSGAFGTVYKGLWIPEGEKVKIPVAIKELREATSPKANKEIL
DEAYVMASVDNPHVCRLLGICLTSTVQLIMQLMPFGCLLDYVREHKDNIGSQYLLNWCVQIAKGMNYLEDRRLVHR
DLAARNVLVKTPQHVKITDFGRAKLLGAEEKEYHAEGGKVPIKWMALESILHRIYTHQSDVWSYGVTVWELMTFGS
KPYDGIPASEISSILEKGERLPQPPICTIDVYMIMVKCWMIDADSRPKFRELIIEFSKMARDPQRYLVIQGDERMH
LPSPTDSNFYRALMDEEDMDDVVDADEYLIPQQGFFSSPSTSRTPLLSSLSATSNNSTVACIDRNGLQSCPIKEDS
FLQRYSSDPTGALTEDSIDDTFLPVPEYINQSVPKRPAGSVQNPVYHNQPLNPAPSRDPHYQDPHSTAVGNPEYLN
TVQPTCVNSTFDSPAHWAQKGSHQISLDNPDYQQDFFPKEAKPNGIFKGSTAENAEYLRVAPQSSEFIGAKETAAA
KFERQHMDSSTSAA-(SEQ ID NO: 33)

What is claimed is:

1. A method of determining if a test compound can interact with a target polypeptide, the method comprising:
(a) preparing a reaction solution enabling contact of a fusion protein within a system comprising:
(i) a test compound or vehicle,
(ii) a denaturant,
(iii) a nuclease donor,
(iv) a nucleic acid substrate, and/or
(v) a signal controller; and
(b) detecting an amount and speed of a cleavage product of the nucleic acid substrate in real time by use of a machine configured to detect fluorescence, generated light, or derivative thereof.

2. The method of claim 1, wherein the machine is a qPCR or a RT-qPCR machine.

3. The method of claim 2, wherein the qPCR or RT-qPCR machine is programmed to mix, initiate, amplify signal, register signal, deconvolute data, analyze data, obtained from the reaction solution in real time, wherein the program comprises any of:
a) mixing reaction solution in batch wherein all ingredients are added from start of mixing, or by injection mode wherein ingredients added at various times;
b) running reaction modules, each with their own commands, such that the data generated in one module can automatically define commands of the next module;
c) running singular or multiplexed reactions;
d) running kinetic series, where various combinations of temperatures and compound doses are tested without prior knowledge of target polypeptide's thermal profile;
e) performing variously timed incubation or incubations with or without agitation or excitation;
f) ramping temperature linearly, step-wise regularly, or irregularly, wherein each ramp step is defined with its own temperature range, speed, repeats and temperature/signal ratios;
g) introducing alternating steps of temperature incubation, signal excitation, and pause;
h) performing data registration, amplification, conversion, deconvolution, and/or analysis;
i) communicating data within a local or remote machine circuit; and/or
j) parsing generated meta-data using programmed analysis methods to identify signal patterns.

4. The method of claim 1, wherein the machine generates target engagement data configured to facilitate the multi-dimensional determination and quantification of engagement between the test compound and the target polypeptide.

5. The method of claim 4, wherein the target engagement data is configured to facilitate identification of binding stoichiometry, target occupancy, residence time, KD, K-on, K-off, and EC50.

6. The method of claim 4, wherein the machine is programmed to generate the target engagement data, wherein the program comprises any of:
a) mixing reaction solution in batch wherein all ingredients are added from start of mixing, or by injection mode wherein ingredients added separately and/or at various times;
b) running reaction modules, each with their own commands, such that the data generated in one module can automatically define commands of the next module;
c) running singular or multiplexed reactions;
d) running kinetic series, where various combinations of temperatures and compound doses are tested without prior knowledge of target polypeptide's thermal profile;
e) performing variously timed incubation or incubations with or without agitation or excitation;
f) ramping temperature linearly, step-wise regularly, or irregularly, wherein each ramp step is defined with its own temperature range, speed, repeats and temperature/signal ratios;
g) introducing alternating steps of temperature incubation, signal excitation, and pause;
h) performing data registration, amplification, conversion, deconvolution, and/or analysis;
i) communicating data within a local or remote machine circuit; and/or
j) parsing generated meta-data using programmed analysis methods in order to find signal patterns.

7. The method of claim 1, wherein the fusion protein comprises:
(a) the target polypeptide and nuclease acceptor,
(b) the target polypeptide and a nuclease, or
(c) the target polypeptide and an N-terminal domain of a nuclease and a first domain allowing for dimerization of the N-terminal domain to a C-terminal domain of the same nuclease fused to a second domain complementary to the domain allowing for dimerization.

8. The method of claim 7, wherein the nuclease acceptor of (a) is an S-tag acceptor peptide and the nuclease donor is an S protein of the RNase S complex.

9. The method of claim 7, wherein the nuclease of (b) is selected from the group consisting of Cas9, Micrococcal nuclease, Rnase H, a non-natural nuclease hybrid such as Cas9-Fok1, and Cpf1/Cas12a.

10. The method of claim 7, wherein the nuclease of (c) is Cas9, the first domain allowing for dimerization is Coh2, and the second domain is DocS.

11. The method of claim 10, wherein the signal controller is far-red light.

12. The method of claim 1, wherein parts or the entirety of reaction solution is/are prepared outside or inside of the machine, such that:
the entirety of reaction is prepared inside the machine;
parts of reaction are prepared outside of the machine, then transferred into the machine; and/or
certain reaction components are injected into the machine at once or sequentially.

13. The method of claim 1, wherein the machine communicates in a circuit with other machines connected locally or remotely.

14. The method of claim 1, wherein the solution is held within a container compatible with real-time fluorescence measuring, and wherein the machine is programmed to read fluorescence signals from the reaction solution in real time.

15. The method of claim 14, wherein the container is a tube or a multi-well plate compatible with real-time fluorescence measuring.

16. The method of claim 15, wherein the multi-well plate comprises a microfluidic chip enabling reaction multiplexing.

17. A non-transitory computer readable medium having instructions thereon, the instructions when executed by a computer causing the computer to perform any of the methods of claim 1.

18. A machine configured to determine if a test compound can interact with a target polypeptide, wherein the machine comprises one or more processors configured by machine readable instructions to:
(a) facilitate preparation of a reaction solution enabling contact of a fusion protein within a system comprising:
(i) a test compound or vehicle,
(ii) a denaturant,
(iii) a nuclease donor,
(iv) a nucleic acid substrate, and/or
(v) a signal controller; and
(b) detect an amount and speed of a cleavage product of the nucleic acid substrate in real time by use of a machine configured to detect fluorescence, generated light, or derivative thereof.

19. The machine of claim 18, wherein the machine is a qPCR or a RT-qPCR machine.

20. The machine of claim 19, wherein the qPCR or RT-qPCR machine is programmed to mix, initiate, amplify signal, register signal, deconvolute data, analyze data, obtained from the reaction solution in real time, wherein the program comprises any of:
a) mixing reaction solution in batch wherein all ingredients are added from start of mixing, or by injection mode wherein ingredients added at various times;
b) running reaction modules, each with their own commands, such that the data generated in one module can automatically define commands of the next module;
c) running singular or multiplexed reactions;
d) running kinetic series, where various combinations of temperatures and compound doses are tested without prior knowledge of target polypeptide's thermal profile;
e) performing variously timed incubation or incubations with or without agitation or excitation;
f) ramping temperature linearly, step-wise regularly, or irregularly, wherein each ramp step is defined with its own temperature range, speed, repeats and temperature/signal ratios;
g) introducing alternating steps of temperature incubation, signal excitation, and pause;
h) performing data registration, amplification, conversion, deconvolution, and/or analysis;
i) communicating data within a local or remote machine circuit; and/or
j) parsing generated meta-data using programmed analysis methods to identify signal patterns.

21. The machine of claim 18, wherein the machine generates target engagement data configured to facilitate the multi-dimensional determination and quantification of engagement between the test compound and the target polypeptide.

22. The machine of claim 21, wherein the target engagement data is configured to facilitate identification of binding stoichiometry, target occupancy, residence time, KD, K-on, K-off, and EC50.

23. The machine of claim 21, wherein the machine is programmed to generate the target engagement data, wherein the program comprises any of:
a) mixing reaction solution in batch wherein all ingredients are added from start of mixing, or by injection mode wherein ingredients added separately and/or at various times;
b) running reaction modules, each with their own commands, such that the data generated in one module can automatically define commands of the next module;
c) running singular or multiplexed reactions;
d) running kinetic series, where various combinations of temperatures and compound doses are tested without prior knowledge of target polypeptide's thermal profile;
e) performing variously timed incubation or incubations with or without agitation or excitation;
f) ramping temperature linearly, step-wise regularly, or irregularly, wherein each ramp step is defined with its own temperature range, speed, repeats and temperature/signal ratios;
g) introducing alternating steps of temperature incubation, signal excitation, and pause;
h) performing data registration, amplification, conversion, deconvolution, and/or analysis;
i) communicating data within a local or remote machine circuit; and/or
j) parsing generated meta-data using programmed analysis methods in order to find signal patterns.

24. The machine of claim 18, wherein the fusion protein comprises the target polypeptide and an S-tag acceptor peptide.

25. The machine of claim 18, wherein parts or the entirety of reaction solution is/are prepared outside or inside of the machine, such that:
the entirety of reaction is prepared inside the machine;
parts of reaction are prepared outside of the machine, then transferred into the machine; and/or
certain reaction components are injected into the machine at once or sequentially.

26. The machine of claim 18, wherein the machine communicates in a circuit with other machines connected locally or remotely.

27. The machine of claim 18, wherein the solution is held within a container compatible with real-time fluorescence measuring, and wherein the machine is programmed to read fluorescence signals from the reaction solution in real time.

28. The machine of claim 27, wherein the container is a tube or a multi-well plate compatible with real-time fluorescence measuring.

29. The machine of claim 28, wherein the multi-well plate comprises a microfluidic chip enabling reaction multiplexing.
